SEQUENCE LISTING (22 sequences)

SEQ ID NO: 1 - protein, 14 aa, synthetic construct
GQEFDEVTADDRKV

SEQ ID NO: 2 - DNA, 55 bp, synthetic construct
  source 1..55: mol_type = other DNA
ccggttgtgctcttcatcttgttgccggcaacaagatgaagagcaccaatttttg

SEQ ID NO: 3 - RNA, 20 nt, synthetic construct
  source 1..20: mol_type = other RNA
gcctccagatgacatagaaa

SEQ ID NO: 4 - RNA, 21 nt, synthetic construct
  source 1..21: mol_type = other RNA
gccttgcattactgctgatat

SEQ ID NO: 5 - RNA, 22 nt, synthetic construct
  source 1..22: mol_type = other RNA
gcgcatggaagaaatagttgaa

SEQ ID NO: 6 - RNA, 22 nt, synthetic construct
  source 1..22: mol_type = other RNA
gcctttagctgtattgtctgaa

SEQ ID NO: 7 - DNA, 20 bp, synthetic construct
  source 1..20: mol_type = other DNA
actgggccaggaatttgacg

SEQ ID NO: 8 - DNA, 21 bp, synthetic construct
  source 1..21: mol_type = other DNA
acgcattccaccaccagttta

SEQ ID NO: 9 - DNA, 21 bp, synthetic construct
  source 1..21: mol_type = other DNA
gcaaacccctattccatgctg

SEQ ID NO: 10 - DNA, 24 bp, synthetic construct
  source 1..24: mol_type = other DNA
gtgtcaaccatggtcatttcttgt

SEQ ID NO: 11 - DNA, 22 bp, synthetic construct
  source 1..22: mol_type = other DNA
ccgggaaactgtggcgtgatgg

SEQ ID NO: 12 - DNA, 25 bp, synthetic construct
  source 1..25: mol_type = other DNA
aggtggaggagtgggtgtcgctgtt

SEQ ID NO: 13 - DNA, 23 bp, synthetic construct
  source 1..23: mol_type = other DNA
taaaaacctggatcggaaccaaa

SEQ ID NO: 14 - DNA, 23 bp, synthetic construct
  source 1..23: mol_type = other DNA
gcattagcttcagatttacgggt

SEQ ID NO: 15 - DNA, 21 bp, synthetic construct
  source 1..21: mol_type = other DNA
ctgcaagagacttccatccag

SEQ ID NO: 16 - DNA, 23 bp, synthetic construct
  source 1..23: mol_type = other DNA
agtggtatagacaggtctgttgg

SEQ ID NO: 17 - DNA, 22 bp, synthetic construct
  source 1..22: mol_type = other DNA
ggtgcctatgtctcagcctctt

SEQ ID NO: 18 - DNA, 23 bp, synthetic construct
  source 1..23: mol_type = other DNA
gccatagaactgatgagagggag

SEQ ID NO: 19 - DNA, 23 bp, synthetic construct
  source 1..23: mol_type = other DNA
aaggtgaagagcatcataaccct

SEQ ID NO: 20 - DNA, 23 bp, synthetic construct
  source 1..23: mol_type = other DNA
tcacgcctttcataacacattcc

SEQ ID NO: 21 - DNA, 20 bp, synthetic construct
  source 1..20: mol_type = other DNA
ggctgtattcccctccatcg

SEQ ID NO: 22 - DNA, 22 bp, synthetic construct
  source 1..22: mol_type = other DNA
ccagttggtaacaatgccatgt

(19) United States
(12) Patent Application Publication
(10) Pub. No.: US 2025/0326825 A1
(43) Pub. Date: Oct. 23, 2025

(54) ANTI-FABP4 ANTIBODIES AND THEIR USES

(71) Applicant: THE HONG KONG POLYTECHNIC UNIVERSITY, Hong Kong (CN)

(72) Inventors: Kin Wah LEE, Hong Kong (CN); Oi Ning LEUNG, Hong Kong (CN)

(21) Appl. No.: 19/184,090

(22) Filed: Apr. 21, 2025

Related U.S. Application Data
(60) Provisional application No. 63/636,219, filed on Apr. 19, 2024.

(51) Int. Cl. C07K 16/18; A61K 39/00; A61P 35/00

(52) U.S. Cl. CPC C07K 16/18; A61P 35/00; A61K 2039/505; C07K 2317/76

(57) ABSTRACT

The antibodies and methods are based, in part, on the understanding that hepatocellular carcinomas, such as metabolic dysfunction-associated steatotic liver disease (MASLD)-induced hepatocellular carcinoma (HCC) can be treated by targeting the FABP4 protein, as these proteins have been shown to play a role in disease initiation and progression.

### CROSS-REFERENCE TO RELATED APPLICATIONS

This application claims the benefit of priority of U.S. provisional application No. 63/636,219, filed 19 Apr. 2024, the contents of it being hereby incorporated by reference in its entirety for all purposes.

### SEQUENCE LISTING

The following application contains a ST.26 sequence listing in computer readable format (CRF), submitted as a text file in .xml format titled “Sequence_Listing_103528US,” created on 17 Apr. 2025, as 20 KB. The content of the CRF is hereby incorporated by reference in its entirety.

### TECHNICAL FIELD

The present invention relates generally to the field of biotechnology. In particular, the present disclosure relates to anti-FABP4 antibodies and methods and uses of the same.

### BACKGROUND

Metabolic dysfunction-associated steatotic liver disease (MASLD)-induced hepatocellular carcinoma (HCC) is an emerging malignancy linked to excessive accumulation of adipose tissue and hepatic fat. However, the mechanisms contributing to its formation, as well as the role of adipocytes in the development of MASLD-induced hepatocellular carcinomas remain largely unknown. In an in vitro co-culture system, differentiated adipocytes were found to enhance cancer stemness and drug resistance in hepatocellular carcinoma through paracrine signalling.

Currently, therapeutic strategies specifically targeting MASLD-induced hepatocellular carcinoma are limited. Standard treatments for virus-associated hepatocellular carcinomas, such as molecular targeted therapies, anti-angiogenic therapies, and immune checkpoint inhibitors, are also employed for MASLD-induced hepatocellular carcinoma but are ineffective.

Thus, there is an unmet need for an effective therapy for treating hepatocellular carcinoma.

### SUMMARY

In one aspect, the present disclosure refers to a monoclonal antibody which binds to fatty acid binding protein 4 (FABP4).

In another aspect, the present disclosure refers to a monoclonal antibody obtained from a hybridoma cell line as disclosed herein.

In yet another aspect, the present disclosure refers to a method for treating hepatocellular carcinoma in a subject, comprising administering an effective amount of a monoclonal antibody to the subject, wherein the monoclonal antibody specifically binds to fatty acid binding protein 4 (FABP4).

### BRIEF DESCRIPTION OF THE DRAWINGS

The invention will be better understood with reference to the detailed description when considered in conjunction with the non-limiting examples and the accompanying drawings, in which:

FIG. 1 shows data indicating that adipocytes enhanced self-renewal, tumorigenicity and drove the drug-resistance in hepatocellular carcinoma (HCC) cells in paracrine manner. (A) shows representative images of intracellular lipids in adipocytes after differentiation. mRNA expression of functional genes of adipocytes (n=3). Scale bar: 100 μm. (B) shows a schematic diagram of co-culturing setup for sphere formation. Representative images of tumourspheres (n=3). Scale bar: 100 μm. (C) shows a schematic diagram showing the workflow of adipocyte condition medium (ADCM) collection, pretreatment of hepatocellular carcinoma (HCC) cells and subcutaneous injection of cells to NOD/SCID gamma (NSG) mice. Representative images of tumours. Scale bar: 1 cm. (D) shows data representing expression of liver CSC markers (n=3 to 4). (E) shows data showing results of the evaluation of migration and invasive abilities of hepatocellular carcinoma cells (n=3). Representative images of stained cells. Scale bar: 250 μm. (F) shows apoptosis of pretreated (DMEM or ADCM) hepatocellular carcinoma cells induced by doxorubicin or sorafenib (n=3). Data was presented as mean±SD. *p<0.05, **p<0.01 & ***p<0.001, two-tailed t-test.

FIG. 2 shows data showing identification of FABP4 as an important secretome conferring CSC properties in hepatocellular carcinoma cells. (A) shows a schematic diagram demonstrating the workflow of adipocyte condition medium (ADCM) collection and conditioned medium from adipocyte-cancer cell co-culturing system (CAACM). (B) shows a Venn diagram illustrating 61 proteins commonly found in both ADCM and CAACM. Nine targets were found to be further upregulated in CAACM. (C) shows the amount of secreted FABP4 in 72 hour-condition medium (CM; n=3). (D) shows the results of a sphere formation assay, demonstrating the role of recombinant human FABP4 (rhFABP4) in regulation of self-renewal ability of hepatocellular carcinoma cells (n=4). Representative images of tumourspheres. Scale bar: 100 μm. (E) shows a schematic diagram of pretreatment of hepatocellular carcinoma cells with rhFABP4 for subcutaneous inoculation using NOD/SCID mice. Representative photos of tumours. Scale bar: 1 cm. (F) shows results of expression of liver CSC markers (n=4). (G) shows the evaluation of the migration and invasive abilities of hepatocellular carcinoma cells (n=4). Representative images of stained cells. Scale bar: 100 μm. (H) shows apoptosis of pretreated hepatocellular carcinoma cells with either PBS (rhFABP4 0 ng/ml) or rhFABP4 induced by doxorubicin or sorafenib was measured (n=4). Data was presented as mean±SD. *p<0.05, **p<0.01 & ***p<0.001, two-tailed t-test.

FIG. 3 shows results indicating that FABP4 deficiency delayed the onset of tumour development in diethylnitrosamine-injected and HFD-induced MASLD-hepatocellular carcinoma mouse model. (A) shows a schematic diagram of the establishment of diethylnitrosamine-injected and high fat diet (HFD)-induced MASLD-hepatocellular carcinoma mouse model. (B) shows representative images of wild-type (WT) and Fabp4 knockout (Fapb4^{-/-}) mice after feeding with HFD for 29 weeks. Scale bar: 1 cm. Expression of FABP4 of the sera and body weight of mice (WT: n=11 mice & Fabp4^{-/-} n=10 mice). (C) shows representative images of mice livers. Graphs showing liver mass and number of tumour nodules. Grey arrows: tumour nodules; Scale bar: 1 cm. (D) shows representative images of H&E staining of the tumours (T: tumour). Expression of AFP of mouse sera. Scale bar: 100 μm. (E) shows expression of FABP4 (light grey) and Perilipin-1 (dark grey) in mouse livers. Scale bar: 100 μm. (F) shows expression of FABP4 (light grey) and Perilipin-1 (dark grey) in human fatty liver tissue. DAPI: nuclei, grey; Scale bar: 50 μm. Data was presented as mean'SD. **p<0.01, ***p<0.001 & ****p<0.0001, two-tailed t-test.

FIG. 4 shows an image depicting the clinical relevance of FABP4 in hepatocellular carcinoma (HCC). (A) shows results indicating that hepatocellular carcinoma patients with high FABP4 had a poorer overall survival rate than those with lower expression in non-tumour in TCGA-LIHC. (B) shows that recombinant human FABP4 (rhFABP4) conferred hepatocellular carcinoma patient-derived organoids with self-renewal ability (n=3, two-tailed t-test). (C) shows the growth of hepatocellular carcinoma patient-derived organoids upon administration of rhFABP4 (n=4, two-tailed t-test). Representative images of organoids. Scale bar: 250 μm. (D) shows the presence of FABP4 mRNA in MASLD patients in GSE193080 (two-tailed t-test). (E) shows FABP4 expression in GSE146049 and 17 paired MASLD-hepatocellular carcinomas of in-house cohort by RNA sequencing (two-tailed t-test). (F) shows the presence of serum FABP4 of MASLD-hepatocellular carcinoma patients. Data was presented as mean±SD. *p<0.05, **p<0.01 & ****p<0.0001.

FIG. 5 shows results indicating that FABP4 activates the PI3K/AKT/Wnt/β-catenin signalling pathway. (A) shows the results of RNA sequencing conducted on PLC/PRF/5 treated with rhFABP4. The gene list of DEGs with a fold change>1.5 was subjected to KEGG pathway enrichment. (B) illustrates the selection criteria of differential expressed genes upon RNA sequencing in tumours from wild-type (WT) and Fabp4^{-/-} mouse liver after being fed a high fat diet (HFD). (C) shows a schematic indicating the top 10 enriched pathways which were identified based on KEGG pathway enrichment analysis in tumour from WT, compared with Fabp4^{-/-}. (D) shows the results of Western blotting of hepatocellular carcinoma cells after treatment with either ADCM or rhFABP4. (E) shows results of examination of transactivating activity of β-catenin after treatment with either ADCM or rhFABP4 for 24 hours (n=3). (F) shows immunohistochemical images of β-catenin in resected mouse livers (n=8 mice). Scale bar: 50 μm. (G) shows the results of limiting dilution sphere analysis showing the role of rhFABP4 in regulation of self-renewal ability upon knockdown of CTNNB1 (n=2, one-sided extreme limiting dilution analysis). Data was presented as mean±SD. *p<0.05, **p<0.01 & ***p<0.001, two-tailed t-test.

FIG. 6 shows results indicating that ITGB1 was identified as a receptor of exogenous FABP4 that mediates cancer stem cell functions by driving the PI3K/AKT/62 -catenin signalling cascade. (A) shows a workflow developed for analysis of potential FABP4-binding proteins on membrane surface of Huh7 using biotinylated rhFABP4. (B) shows surface proteins detected with ≥2 unique peptides. (C) is a diagram showing interactions between potential FABP4 binding proteins and main players in Wnt/β-catenin signalling. (D) shows a Kaplan-Meier curve illustrating that, among hepatocellular carcinoma patients with MASLD as risk factor in The Cancer Genome Atlas Liver Hepatocellular Carcinoma (TCGA-LIHC), patients with high ITGB1 had poorer overall survival rate than those with low ITGB1 (log rank test). (E) shows correlation graphs indicating that, according to GSE192959, GSE193080 and GSE193066, FABP4 was positively correlated with ITGB1 in MASLD-related hepatocellular carcinoma patients and MASLD patients at high risk of hepatocellular carcinoma (Pearson correlation). (F) shows results of reciprocal coimmunoprecipitation showing the interaction between exogenous FABP4 and ITGB1 (n=2). (G) shows the results of a Western blot analyses of shITGB1 hepatocellular carcinoma (HCC) cells (i.e., cells in which ITGB1 had been silenced using shRNA) upon treatment with rhFABP4. (H) shows results of examination of transactivating activity of β-catenin in shITGB1 hepatocellular carcinoma cells after treatment of rhFABP4 for 24 hours (n=3, two-tailed t-test). (I) shows the results of a limiting dilution sphere analysis, showing the role of ITGB1 in regulation of FABP4-driven self-renewal ability (n=2, one-sided extreme limiting dilution analysis). (J) is a column graph showing data measuring apoptosis of ITGB1-silenced hepatocellular carcinoma cells pretreated with PBS (rhFABP4 0 ng/ml), rhFABP4 (100 ng/ml), doxorubicin or sorafenib for 48 hours. (n=4−5, two-tailed t-test). (K) is a column graph showing the effect of rhFABP4 on cell migration and invasion upon silencing of ITGB1 (n=4−6, two-tailed t-test). (L) shows images of xenograft tumours of ITGB1-knockdown and control hepatocellular carcinoma cells pretreated with either PBS or rhFAPB4 at 100 ng/ml for 24 hours, which had been subcutaneously inoculated into nude mice. Scale bar: 1 cm. (M) shows graphs depicting tumour masses (n=5 mice per group, one-way ANOVA followed by Tukey's test). Data was presented as mean±SD. * p<0.01, **p<0.01, ***p<0.001 & ****p<0.0001.

FIG. 7 shows data indicating the effect of the anti-FABP4 monoclonal antibody (mAb) on cancer stem cell (CSC) properties and tumour suppression in a MASLD-hepatocellular carcinoma mouse model. (A) shows images of a Western blot showing the specificity of the FABP4 monoclonal antibody (mAb). (B) shows the results of a limiting dilution sphere analysis of the effect of rhFABP4 on hepatocellular carcinoma cells following treatment with FABP4 mAb (n=2, one-sided extreme limiting dilution analysis). (C) shows images of the effect of ablation of FABP4 on migration and invasion abilities of cells treated with rhFABP4 (n=3, two-tailed t-test). Representative images of stained cells. Scale bar: 200 μm. (D) shows column graphs representing apoptosis of hepatocellular carcinoma cells treated with either PBS (0 ng/mL rhFABP4) or rhFABP4 (100 ng/ml) or in combination with the FABP4 mAb induced by doxorubicin or sorafenib for 48 hours (n=4, two-tailed t-test). (E) shows the workflow developed for the pretreatment of hepatocellular carcinoma cells with rhFABP4 and anti-FABP4 mAb prior to subcutaneous inoculation in nude mice. (F) shows images of xenograft tumours. Scale bar: 1 cm. (G) is a graph showing the mass of xenografts (one-way ANOVA followed by Tukey's test). (H) is a schematic of the experimental design for orthotopic MASLD-hepatocellular carcinoma mouse model and treatment with FABP4 mAb. (I) is a graph showing the amount of serum FABP4 after 13 weeks of high-fat diet (HFD) compared with those after standard diet (STD) before orthotopic implantation (two-tailed t-test). (J) shows image of hepatocellular carcinoma (HCC) tumours. Untreated hepatocellular carcinoma tumours of the standard diet (STD) group were included. Scale bar: 1 cm. (K) shows a graph depicting luciferase signal intensity of the livers (STD: n=4 mice & HFD: n=6 mice per group, one-way ANOVA followed by Tukey's test). (L) shows a graph depicting the liver/body weight ratio of the mice (one-way ANOVA followed by Tukey's test). (M) shows immunohistochemical images of proliferating cell nuclear antigen (PCNA) in resected tumours (n=5 random fields, one-way ANOVA followed by Tukey's test). Scale bar: 5 μm. (N) shows expression of pAKT(Ser473) (white), pGSK3β(Ser9) (light grey) and β-catenin (dark grey) in resected tumours (n=10 random fields, one-way ANOVA followed by Tukey's test). DAPI: nuclei, grey. Data was presented as mean±SD. *p<0.05. **p<0.01, ***p<0.001 & ****p<0.0001.

FIG. 8 shows results indicating that the secretion of FABP4 in adipocytes was stimulated by conditioned medium from adipocyte conditioned medium (ADCM)-incubated hepatocellular carcinoma cells. (A) shows a schematic diagram showing the workflow of stimulated adipocyte conditioned medium (CAACM) collection. The tumorsphere formation of PLC/PRF/5 was enhanced by ADCM when compared to DMEM control, which was further elevated by CAACM (n=3 independent experiments, *p<0.05 & **p<0.01, two-tailed t-test). Scale bar=100 μm. (B) shows a schematic diagram showing the workflow of conditioned medium collection from stimulated adipocytes or hepatocellular carcinoma cells. (C) shows the level of secreted FABP4 in conditioned media detected using ELISA (n=2 independent experiments). Data was presented as the mean±SD.

FIG. 9 shows results indicating that inhibition of FABP4 in adipocyte conditioned medium (ADCM) by BMS-309403 attenuated the cancer stem cell (CSC) properties of hepatocellular carcinoma cells. (A) shows the results of an in vitro tumoursphere formation assay which showed that BMS-309403 at 1 μM suppressed the enhancing self-renewal ability of ADCM conferred to PLC/PRF/5 (n=3 independent experiments). Representative images showing the tumourspheres. Scale bar: 100 μm. (B) shows expression of liver CSC marker CD90 measured by flow cytometry analysis. (n=3 independent experiments). (C) shows results evaluating the migration and invasive abilities of hepatocellular carcinoma cells by uncoated (top) and Matrigel-coated transwell (bottom) assays, respectively (n=3 independent experiments). Representative images of stained cells were shown. Scale bar: 250 μm. (D) shows amount of apoptosis measured due to cotreatment of ADCM with BMS-309403 hepatocellular carcinoma cells induced by doxorubicin (1 μg/mL) or sorafenib (6 μM), Measurements were obtained using Annexin-V PI staining (Doxorubicin treatment: n=3 while sorafenib treatment: n=4 independent experiments). Data was presented as the mean±SD. *p<0.05, ** p<0.01, ***p<0.001 & ****p<0.0001, two-tailed t-test.

FIG. 10 shows results indicating that genetic loss of Fabp4 dampened steatosis and inflammation of the livers upon diethylnitrosamine-high fat dict. (A) shows assessment of steatosis of mouse livers from WT and Fabp4^{-/-} using H&E staining (WT n=9 & Fabp4^{-/-}n=10 mice) and BODIPYTM493/503 staining (n=3 mice). Representative H&E and BODIPYTM493/503 images were shown. The tissue samples were counterstained with either haematoxylin or DAPI (grey). Scale bar: 10 μm and 50 μm. (B) shows the assessed lobular inflammation score. Scale bar: 10 μm. (C) shows measurement of the expression of pro-inflammatory genes Tnf, Il6 and Ccl2 in the livers (n=7 mice). (D) shows results of the examination of the level of F4/80 (white) macrophages in the livers (n=3 mice). The tissue samples were counterstained with DAPI (grey). Scale bar: 50 μm. (E) shows the results of multiplex immunohistochemistry (IHC) staining on M1 and M2 macrophages (M∅) performed using combination of CD68 (white)/CD86 (light grey) or CD68 (white)/CD206 (dark grey) (n=4 mice). Scale bar: 50 μm. Number of positive cells was quantified and expressed as M1/M2 M∅ ratio. (F) depicts a schematic diagram showing the long-term feeding of high-fat diet (HFD) in mice. Fabp4 level of livers and visceral fat was examined (STD n=6 & HFD n=4 mice). Representative images of livers and visceral fat (dotted circle) were presented. Scale bar: 1 cm. (G) shows a presentation of RNA expression of human FABP4 in different cell types from The Human Protein Atlas (cutoff of nTPM>10000). Data was presented as mean±SD. *p<0.05, **p<0.01 & ****p<0.0001, two-tailed t-test.

FIG. 11 shows results illustrating the effect of adipocyte-conditioned medium (ADCM) on hepatocellular carcinoma patient-derived organoids. (A) shows the results of adipocyte conditioned medium (ADCM) enhanced cell invasiveness of hepatocellular carcinoma (HCC) patient-derived organoids in matrigel-coated transwell assay (n=4 independent experiments). Scale bar: 250 μm. (B) shows representative images of organoids upon treatment of ADCM. The growth of hepatocellular carcinoma patient-derived organoids was enhanced upon administration of ADCM for 6 days using Cell-Titre-Glo assay (n=4 independent experiments). Scale bar: 250 μm. Data was presented as the mean±SD. *p<0.05, two-tailed t-test.

FIG. 12 shows results indicating biotinylation of recombinant FABP4 protein (rhFABP4) for cell surface target binding identification using mass spectrometry. (A) shows a Western blot analysis showing the successful detection of FABP4 protein after biotinylation. (B) shows the detection of biotinylated FABP4 after capturing of streptavidin. (C) shows data indicating that the function of rhFABP4 was not affected upon biotinylation, as demonstrated by the upregulation of pGSK3βSer9) and β-catenin in Western blot analyses.

FIG. 13 shows data indicating that FABP4 expression positively correlated with CTNNB1 in MASLD-related hepatocellular carcinoma (HCC) patients and MASLD patients at high risk of hepatocellular carcinoma (HCC). According to the GSE192959 and GSE193066 datasets, FABP4 was positively correlated with CTNNB1 in MASLD-related hepatocellular carcinoma patients and MASLD patients at high risk of hepatocellular carcinoma (R=0.5, p=0.00029 & R=0.3, p=0.021; Pearson correlation).

FIG. 14 shows data of the generation of anti-human FABP4 monoclonal antibody based on the functional characterization of the mouse ascites. (A) shows results of the detection efficiency of the six (6) ascites examined using coupling peptide in dot blot assay. Representative dot blot results were shown. (B) shows the results of cotreatment of rhFABP4 with ascites #1 to #6, respectively, at 10 μg/mL for 24 hours using PLC/PRF/5. Western blot analyses showed that ascites #6 repressed the upregulation of β-catenin. Ascites #6 was chosen for functional characterization. (C) shows results indicating that the effects of rhFABP4 at 100 ng/ml in cell migratory and invasive abilities were abolished by treatment of ascites #6 at 2 μg/mL when compared with normal IgG control (n=4 independent experiments). Scale bar: 200 μm. (D) shows results indicating that the enhanced self-renewing ability by rhFABP4 was abrogated by cotreatment with ascites #6 at 1 μg/mL (n=4 independent experiments). Scale bar: 200 μm. (E) shows results showing that drug resistance conferred by rhFABP4 was rescued by ascites #6 upon treatment of either doxorubicin (1.5 μg/mL) or sorafenib (6 μM) (n=5 independent experiments). Data was presented as the mean±SD. *p<0.05 & **p<0.01, two-tailed t-test.

FIG. 15 shows results depicting the effect of anti-FABP4 monoclonal antibody on body weight of the mice. No notable change in body weight was observed after treatment of anti-FABP4 mAb ranging from 400 μg to 1200 μg (n=6 mice per group). Data was presented as the mean±SD.

FIG. 16 shows data indicating neutralisation of FABP4 by the monoclonal antibody as disclosed herein (referred to as an anti-FABP4 antibody, or 3I19-1) in a MASLD-hepatocellular carcinoma mouse model suppressed the tumour growth through modulating of immune environment in the liver. (A) shows results indicating that accumulation of neutral lipid droplets in the livers was reduced upon administration of anti-FABP4 monoclonal antibody at 400 μg (n=3 mice). Representative BODIPYTM493/503 images were shown (NT: non-tumour, T: tumour). The tissue was counterstained with DAPI (grey). Intensity of BODIPYTM493/503 was quantified using ImageJ. Scale bar: 100 μm. (B) shows results of examination of the expression of pro-inflammatory genes Tnf, Il6 and Ccl2 in mouse livers (n=5 mice). (C) shows results of the examination of the level of F4/80 macrophages in the livers (n=4 mice). The tissue was counterstained with DAPI (grey). Positive staining was quantified using ImageJ. Scale bar: 50 μm. (D) shows the results of multiplex IHC staining on M1 and M2 macrophages (M∅) performed using combination of CD68 (white)/CD86 (light grey) or CD68 (white)/CD206 (dark grey) (n=6 mice). Scale bar=50 μm. Tissue was counterstained with DAPI (grey). Number of positive cells was quantified using ImageJ and expressed as M1/M2 M∅ ratio. Data was presented as mean±SD. *p<0.05, **p<0.01 & ****p<0.0001, two-tailed t-test.

### DETAILED DESCRIPTION

Liver cancer ranks as the third deadliest malignancy worldwide. Hepatocellular carcinoma (HCC), which accounts for approximately 90% of primary liver cancers, typically arises due to chronic liver disease caused by hepatitis B virus (HBV) or hepatitis C virus (HCV) infection, alcohol-associated liver diseases or the increasingly common metabolic dysfunction-associated steatotic liver disease (MASLD; formerly known as non-alcoholic fatty liver disease (NAFLD)). MASLD has not only become the most common chronic liver disease but also a public health crisis, give that it has a global prevalence of approximately 25%. MASLD is diagnosed as such if hepatic steatosis is accompanied by either obesity or overweight, type 2 diabetes mellitus or evidence of metabolic dysregulation. Metabolic-associated steatohepatitis (MASH), a prominent subtype of MASLD, is diagnosed when MASLD presents with inflammatory activity and hepatocyte injury in a fatty liver tissue. MASH itself is understood to be a spectrum of progressive liver disorders that leads to cirrhosis and end-stage liver failure and confers an increasing risk of the development of hepatocellular carcinoma (HCC). With the increasing incidence of obesity and diabetes, MASH is undoubtedly becoming the fastest growing aetiology of hepatocellular carcinoma. Although studies suggest that the prevalence of hepatocellular carcinoma has increased 11.5-fold in patients with MASH, the pathogenesis and molecular mechanisms underlying the onset of these conditions are still poorly understood.

MASLD is a condition characterized by liver fat accumulation in individuals with diabetes, obesity, high blood pressure, or high cholesterol who consume little to no alcohol. Without being bound by theory, it is thought that visceral adipose tissue and hepatic fat contribute to the development of MASLD-induced hepatocellular carcinoma. A link between mouse liver cancer stem cells (CSCs) and obesity-mediated steatohepatitis has been observed. Nonetheless, the link between hepatocellular carcinoma cell stemness and the interplay of adipocytes in MASLD is unclear.

As used herein, the term “stemness” refers to the molecular processes underlying the defining properties of a stem cell of self-renewal and the generation of differentiated progeny.

Fatty acid-binding protein 4 (FABP4) is shown to be preferentially secreted by adipocytes, and the application of recombinant FABP4 is shown to further augment the cancer stem cell (CSC) properties of hepatocellular carcinoma cells. Fabp4^{-/-} mice exhibited a delay in the progression of MASLD-hepatocellular carcinoma, which correlates with the increased hepatocellular carcinoma risk observed in MASLD patients with elevated FABP4 expression. Mass spectrometry analysis identified integrin beta 1 (ITGB1) as a binding partner of FABP4. This data, together with a downregulation of the Wnt/β-catenin pathway in Fabp4^{-/-} mice tumours, shows that FABP4 augments liver cancer stem cell functions by activating PI3K/AKT/β-catenin signalling via ITGB1. The anti-FABP4 neutralising antibody, as described herein, is shown to inhibited FABP4-driven cancer stem cell functions and to suppress MASLD-induced hepatocellular carcinoma. Summarily, adipocyte-derived FABP4 is shown to play a role in the development of MASLD-induced hepatocellular carcinoma, and targeting the ITGB1/PI3K/AKT/β-catenin signalling cascade offers an approach to treating this aggressive disease.

As used herein, the term “neutralising”, in the context of the antibodies disclosed herein, refers to the ability of the antibody to inhibit the function and/or activity of FABP4. This can be done, for example, by, blocking FABP4s interaction with a FABP4 receptor and thus disrupting its downstream pathway.

Thus, in one example, the antibody disclosed herein is a neutralising antibody. In another example, the antibody is a monoclonal, neutralising antibody.

Here, adipocytes differentiated from human visceral preadipocytes were used and showed that the adipocyte secretome was involved in promoting the function of liver cancer stem cells, while the cancer stem cell-enhancing effect was further enhanced upon co-culturing with hepatocellular carcinoma cells. Orbitrap analysis showed that fatty acid binding protein 4 (FABP4) plays a role in this mechanism. Functional studies showed that secreted FABP4 augments hepatocellular carcinoma tumour growth by modulating the PI3K/AKT/β-catenin signalling pathway through its interaction with ITGB1. This data is consistent with the delayed development of MASLD-induced hepatocellular carcinoma shown in Fabp4^{-/-} mice. A monoclonal neutralising antibody was developed to specifically target FABP4, which not only blocked FABP4-driven cancer stem cell functions, but also suppressed tumour formation in a MASLD-hepatocellular carcinoma mouse model, indicating the targetability of FABP4 for the treatment of MASLD-induced hepatocellular carcinoma.

The data shown herein shows the involvement of adipocytes in the liver in the development and progression of MASLD-induced hepatocellular carcinoma, which is facilitated by the selective secretion of FABP4. Results shown herein provide mechanistic insights by identifying ITGB1 as the receptor that regulates this process, highlighting it as a way of targeting its signalling pathway as a therapeutic approach for treating MASLD-induced hepatocellular carcinoma.

Using a co-culture system in which differentiated adipocytes were grown with hepatocellular carcinoma cells, adipocytes were found to enhance the self-renewal ability of hepatocellular carcinoma cells through indirect paracrine secretion. hepatocellular carcinoma cells pre-incubated with conditioned medium (CM) from adipocytes showed enhanced liver cancer stem cell properties, including, but not limited to, self-renewal, tumorigenicity, invasiveness, and resistance to doxorubicin and sorafenib. Secretome profiling revealed that FABP4 is preferentially secreted by adipocytes and its level was further augmented when co-cultured with hepatocellular carcinoma cells. Concurrently, recombinant FABP4 was shown to enhance the cancer stem cell properties of hepatocellular carcinoma cells.

As used herein, the term “metabolic dysfunction—associated steatotic liver disease” (MASLD), refers to an abnormal accumulation of fat in the liver in the absence of secondary causes of fatty liver, such as, for example, significant alcohol use, viral hepatitis, or medications that can induce fatty liver. This was also previously the definition of non-alcoholic fatty liver disease (NAFLD). However, the term “metabolic dysfunction—associated steatotic liver disease” (MASLD), which was adopted in the field in 2023 and replaces the term “NAFLD”, allows for other conditions to be present and focuses on the metabolic abnormalities contributing to the disorder. MASLD encompasses a continuum subtypes of liver abnormalities, for example, from “metabolic dysfunction—associated steatotic liver” (MASL, also referred to as simple steatosis) to “metabolic dysfunction—associated steatohepatitis” (MASH) as another example. These diseases begin with hepatic steatosis (also known as fatty accumulation in the liver). A liver can remain fatty without disturbing liver function (and would then be considered to fall under the term MASL), but by various mechanisms and possible exacerbations to the liver, it can also progress into steatohepatitis (MASH), a state in which steatosis is combined with inflammation and sometimes fibrosis. MASH can further lead to complications, such as, but not limited to, cirrhosis and hepatocellular carcinoma.

### Adipocytes, not Preadipocytes, Enhance Liver Cancer Stemness and Drug Resistance in Hepatocellular Carcinoma Cells

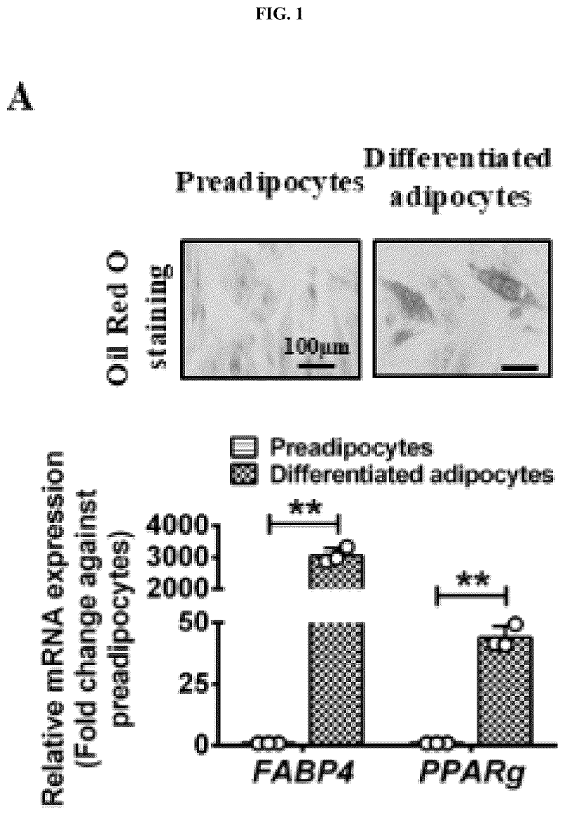

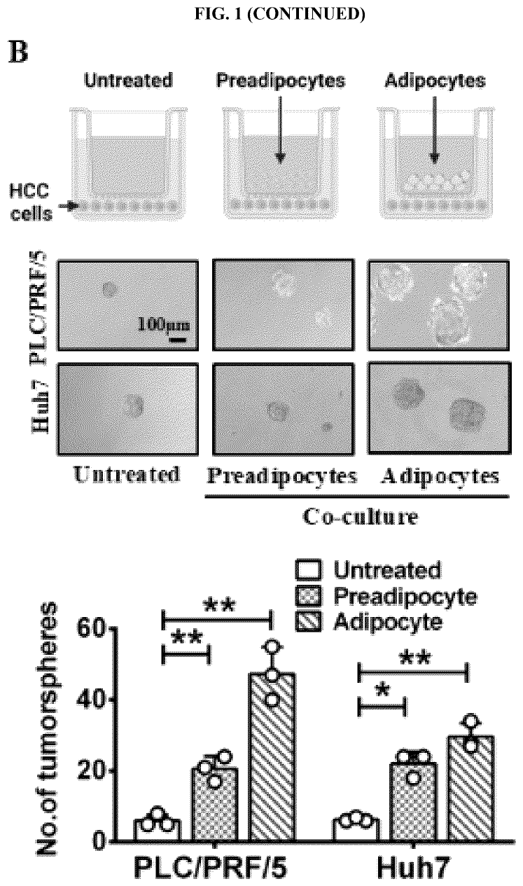

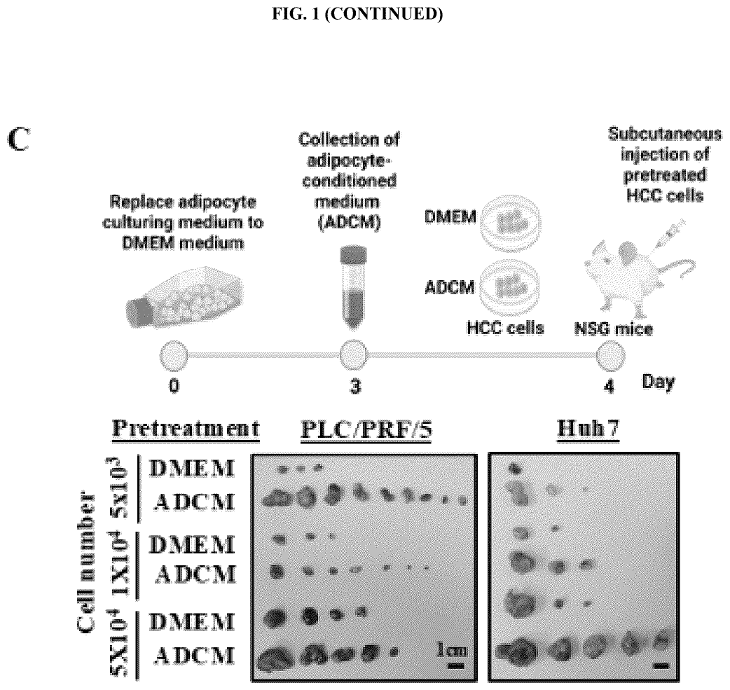

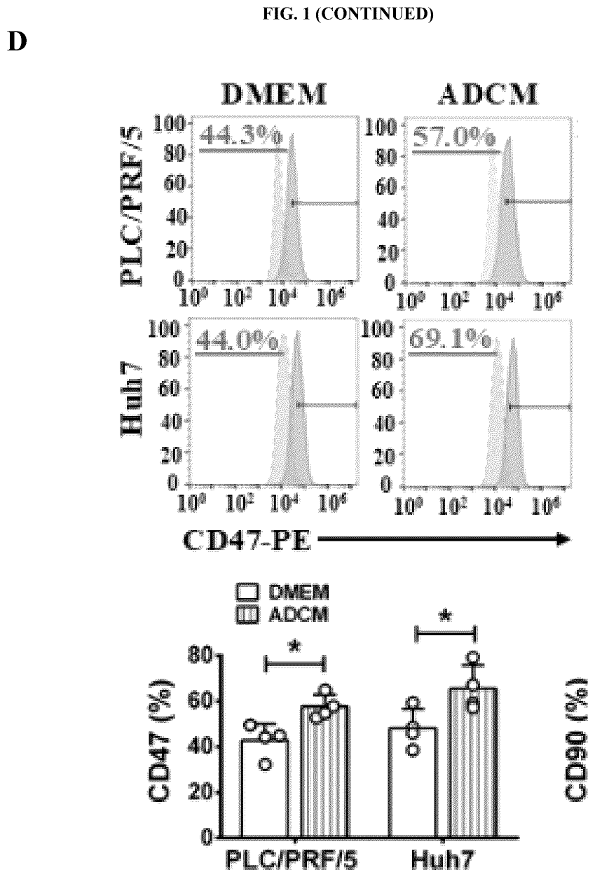

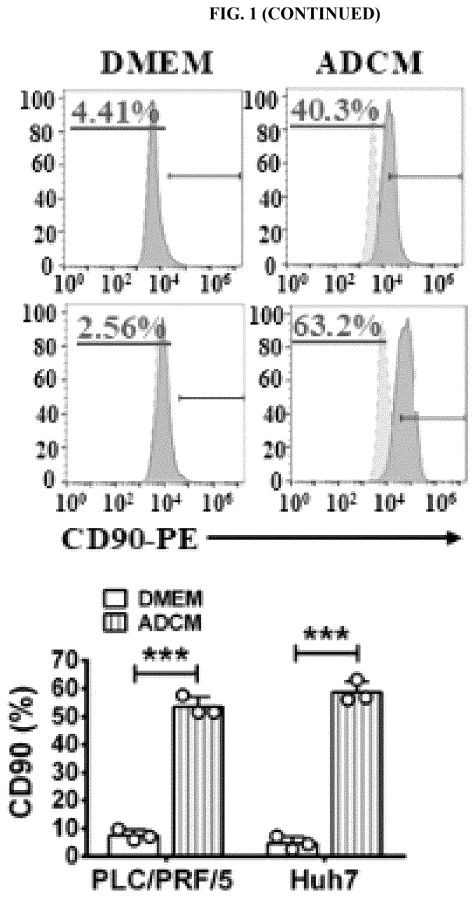

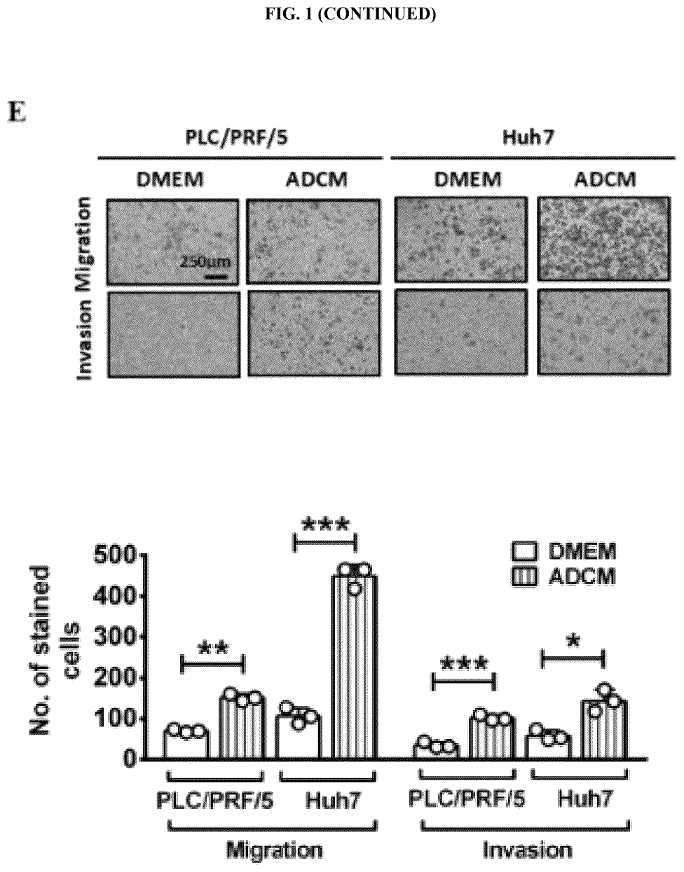

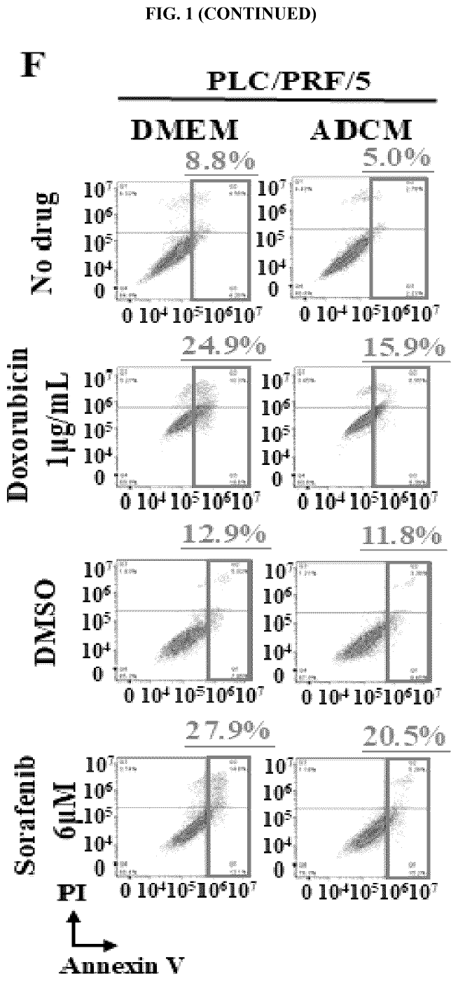

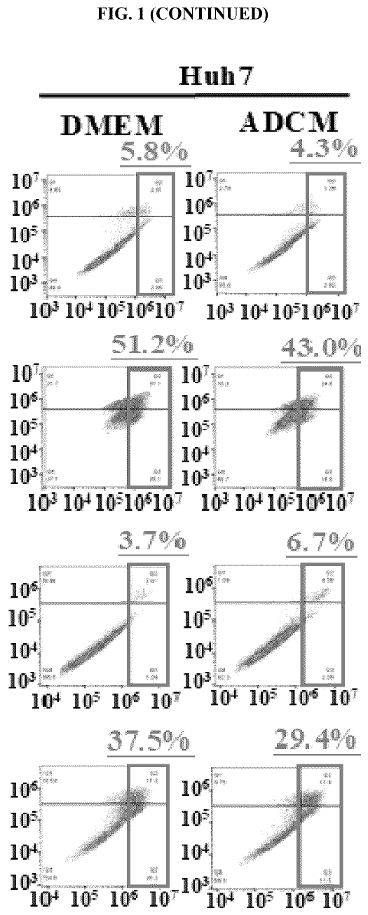

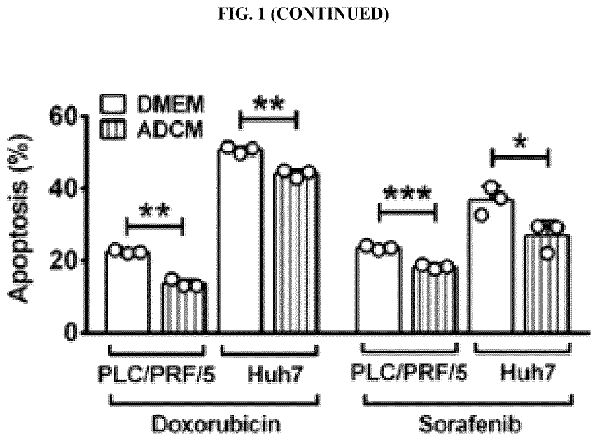

An increase in visceral fat results in increased production of proinflammatory adipokines, and this dysregulation of adipokines via visceral adipose tissue contributes to the development of MASH. Thus, human adipocytes were established in culture by in vitro differentiation of commercially available human visceral preadipocyte cells. The establishment of functional visceral adipocytes was confirmed by the presence of intracellular lipids and the upregulation of specific genes related to adipocyte differentiation and adipogenesis, including, but not limited to, FABP4 and PPARG, compared to their preadipocyte counterparts (FIG. 1A). Co-culturing these adipocytes with hepatocellular carcinoma (HCC) cells in a transwell experimental setup promoted self-renewal of the hepatocellular carcinoma cells compared to that of untreated hepatocellular carcinoma cells or hepatocellular carcinoma cells co-cultured with undifferentiated preadipocytes (FIG. 1B). This result indicates that adipocytes regulate liver cancer stem cells via paracrine secretion. To verify this, conditioned medium of adipocytes (ADCM) was collected for analysis using an in vivo limiting dilution assay. Pre-treatment of hepatocellular carcinoma cells with ADCM was shown to increase the size and number of hepatocellular carcinoma tumours (FIG. 1C and Table 1). In addition, pre-treatment of hepatocellular carcinoma cells with ADCM also increased the expression of liver cancer stem cell markers, including, for example, CD47 and CD90 (FIG. 1D); migration and invasion (FIG. 1E); and drug resistance to doxorubicin and sorafenib (FIG. 1F). All these results show that adipocyte secretomes exert cancer stem cell-enhancing effects in a paracrine manner.

### Mass Spectrometry Profiling Revealed that FABP4 is Preferentially Secreted by Adipocytes in co-Culture With Hepatocellular Carcinoma Cells

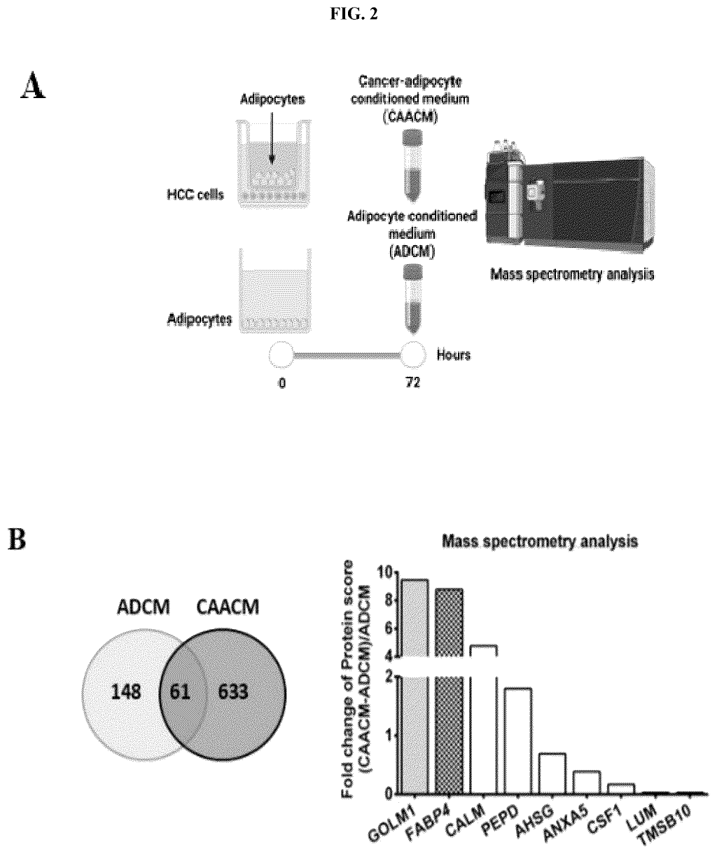

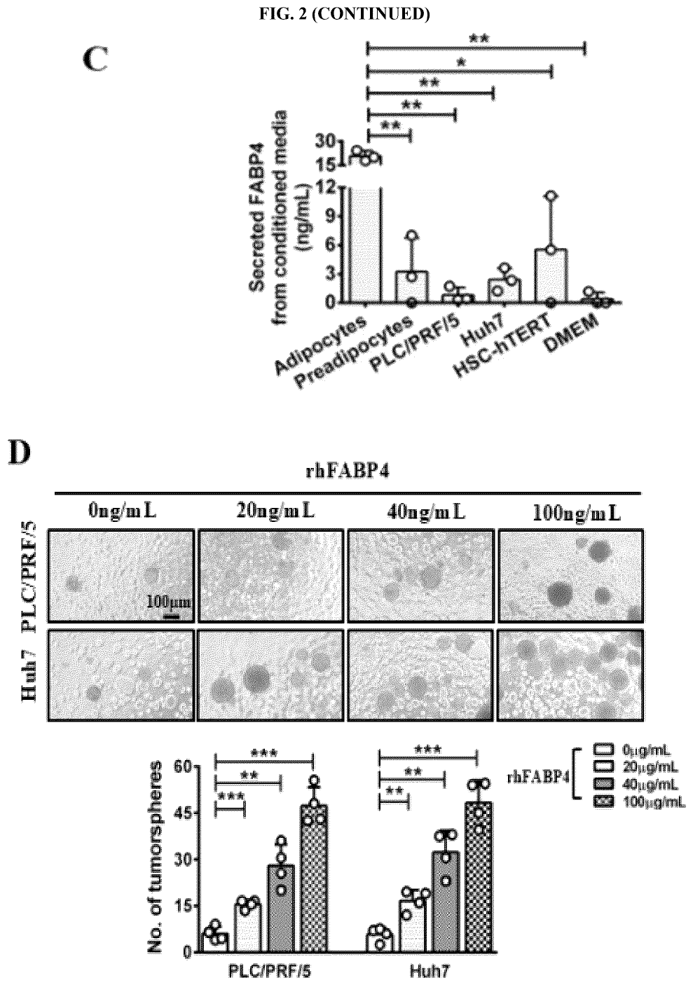

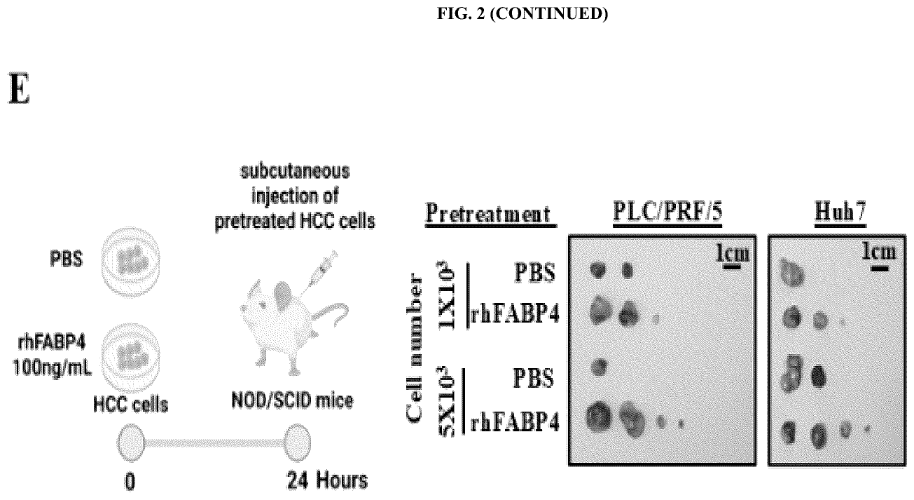

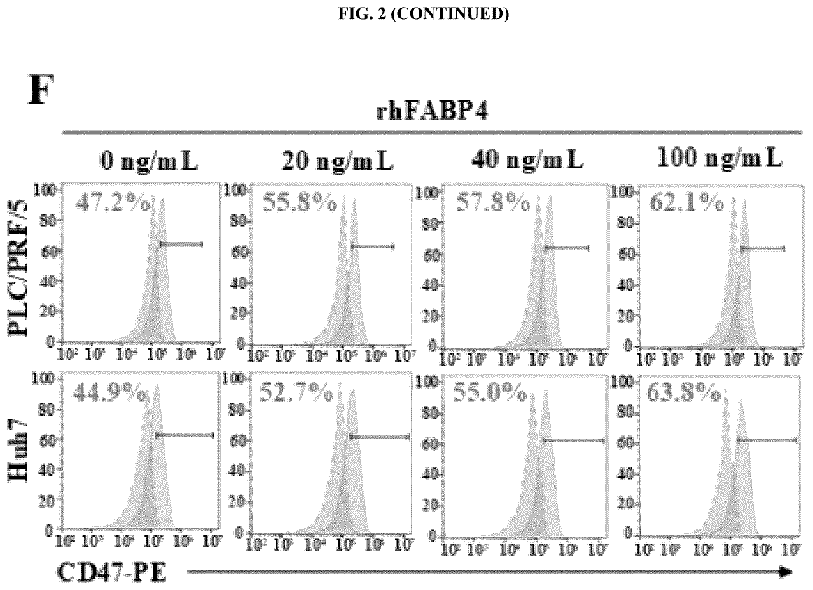

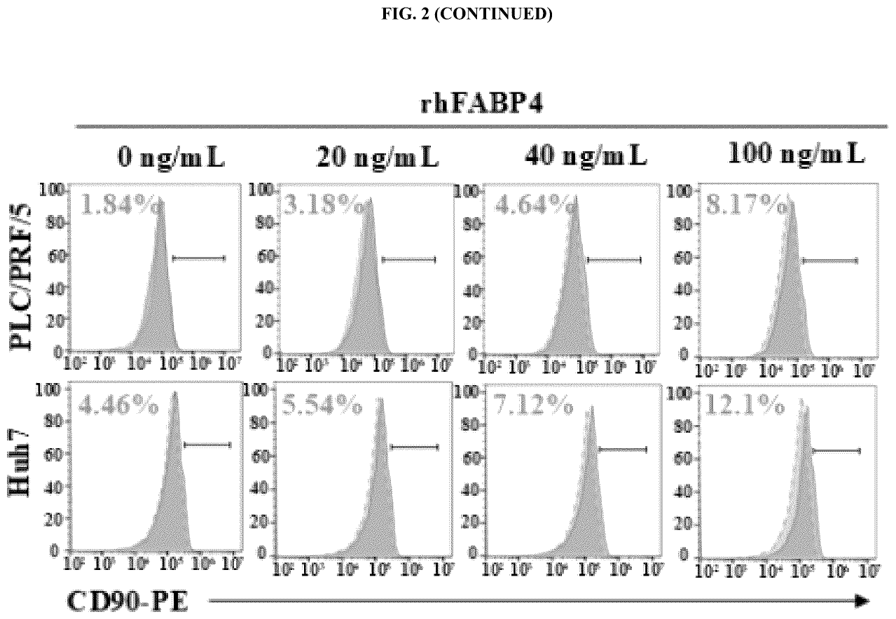

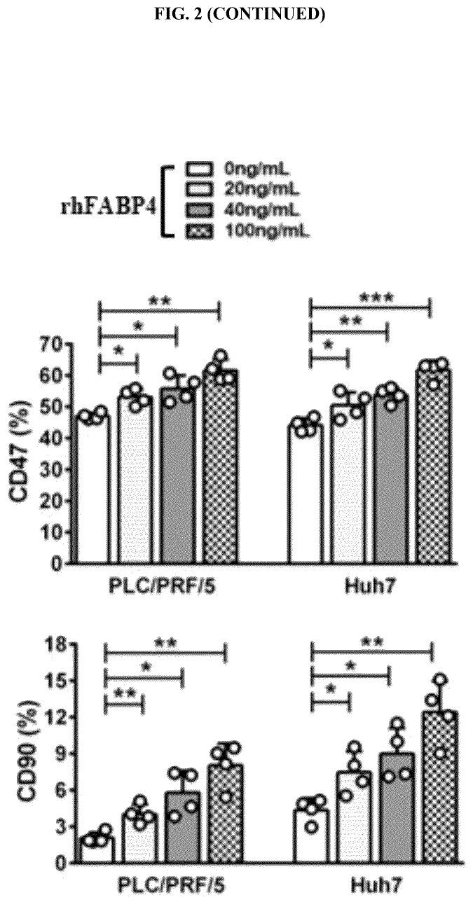

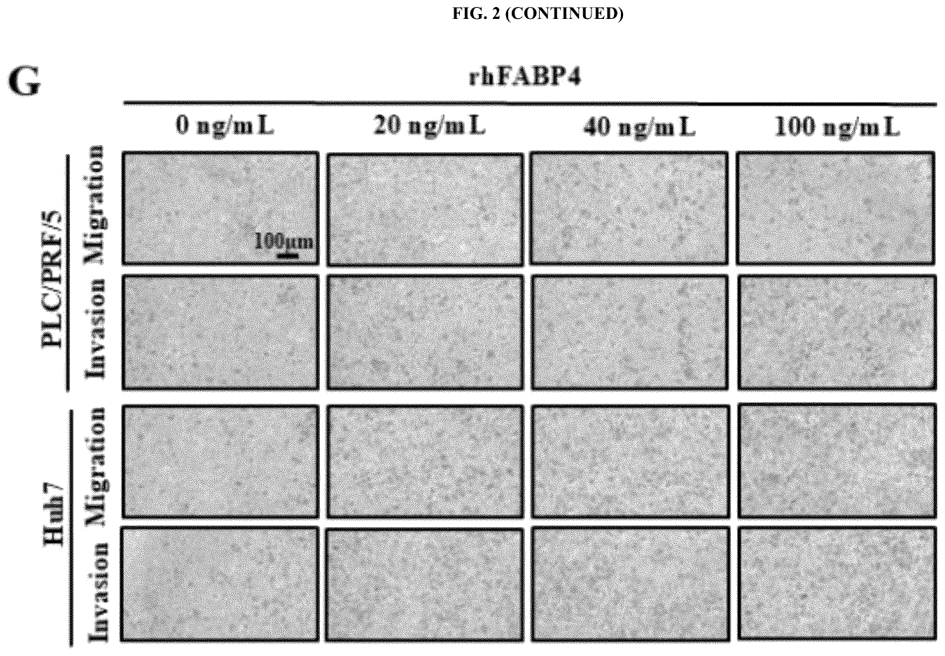

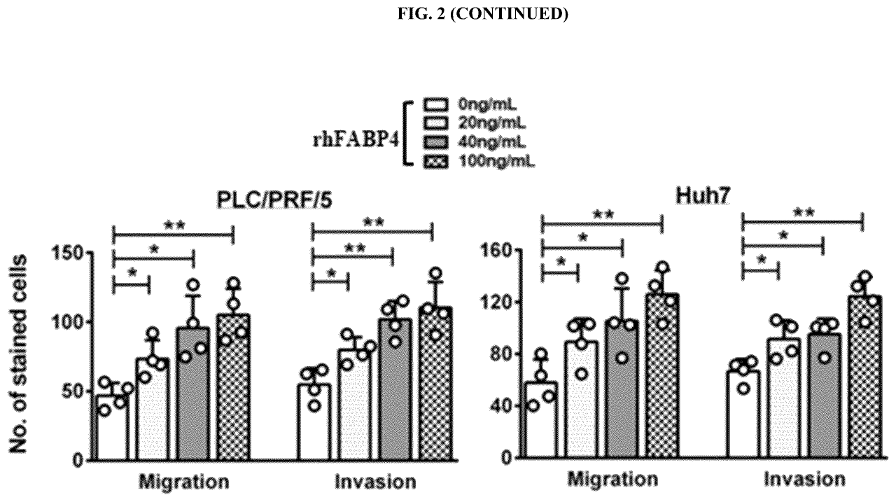

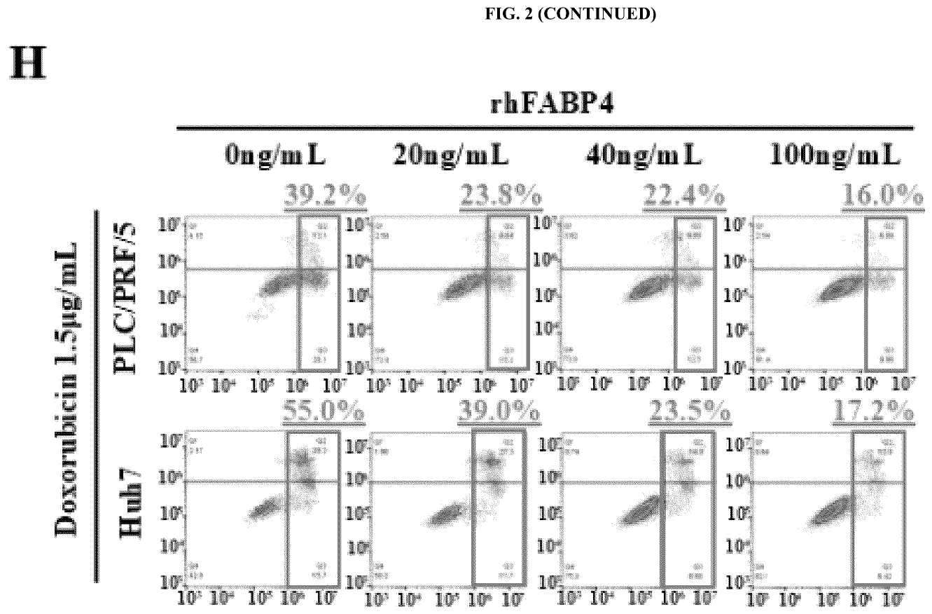

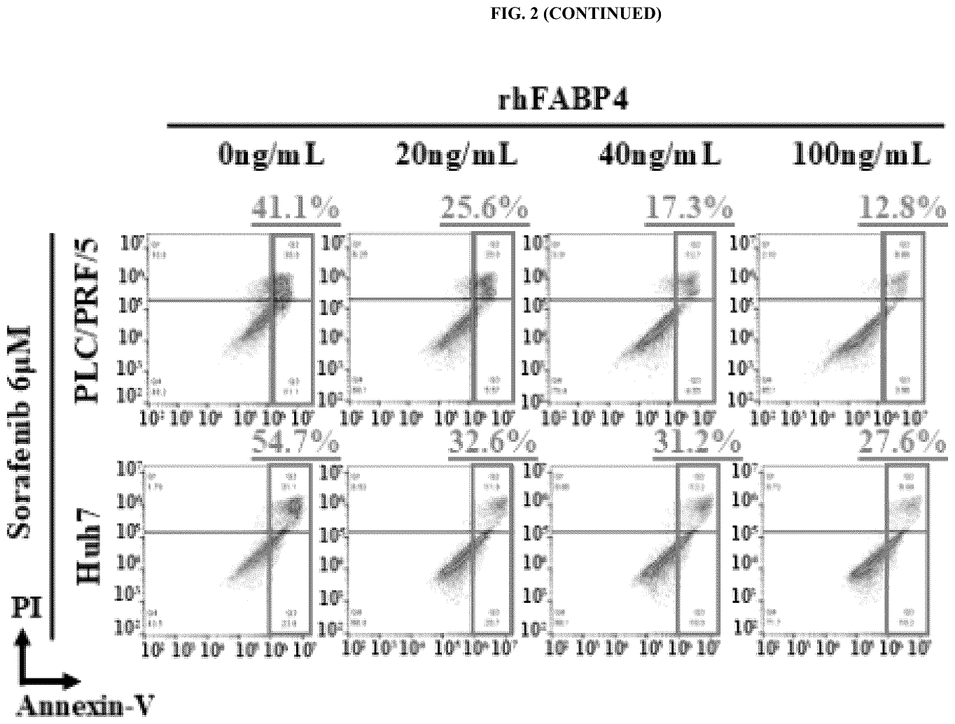

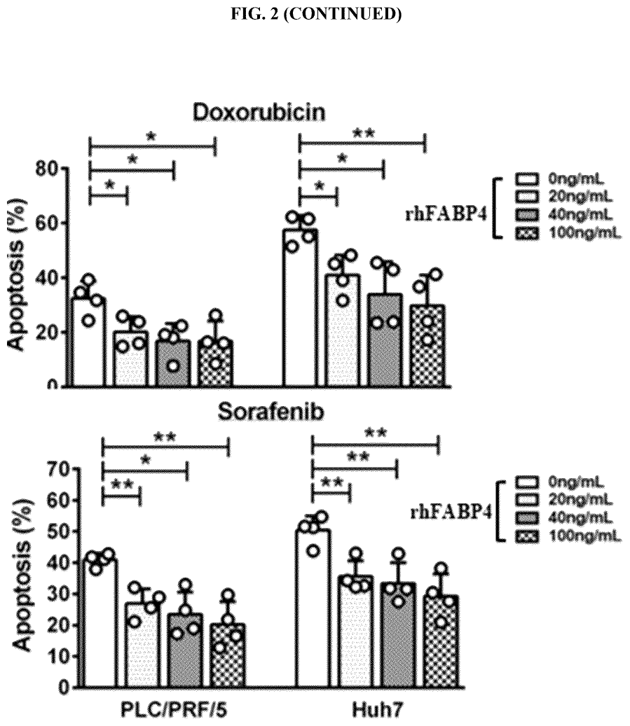

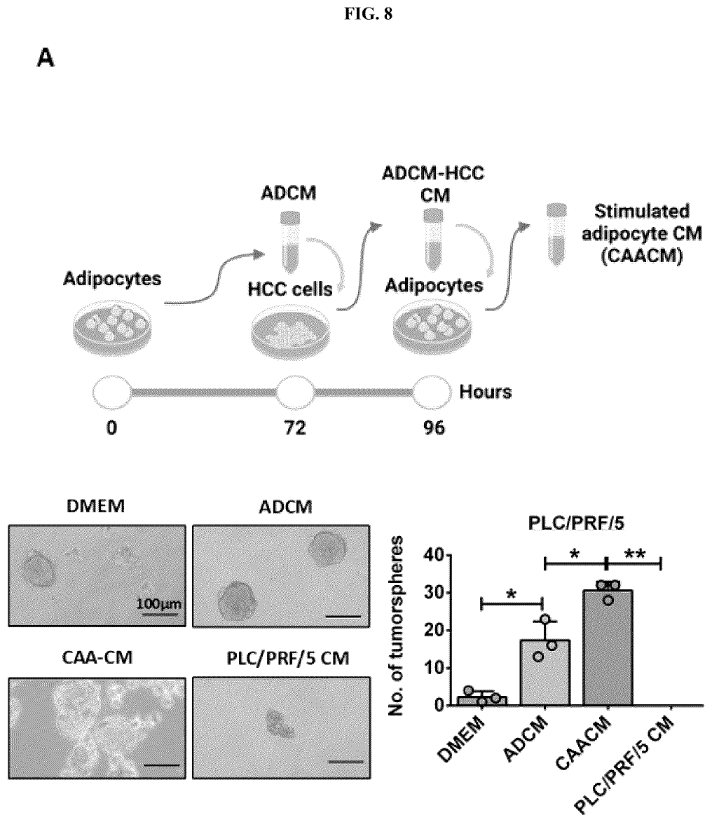

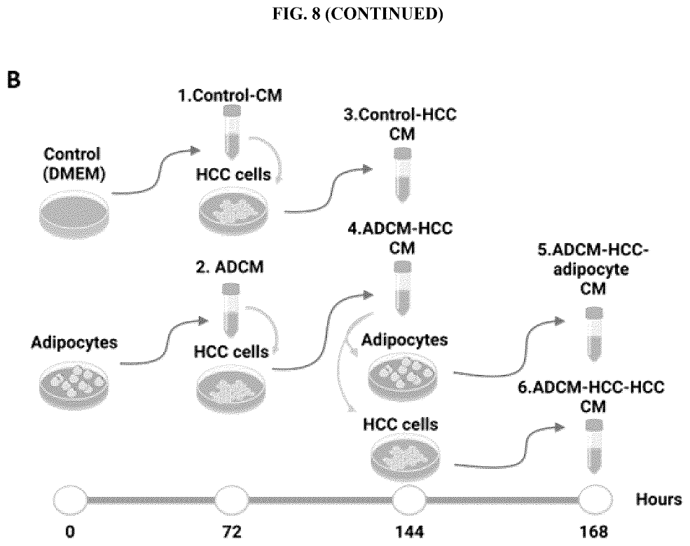

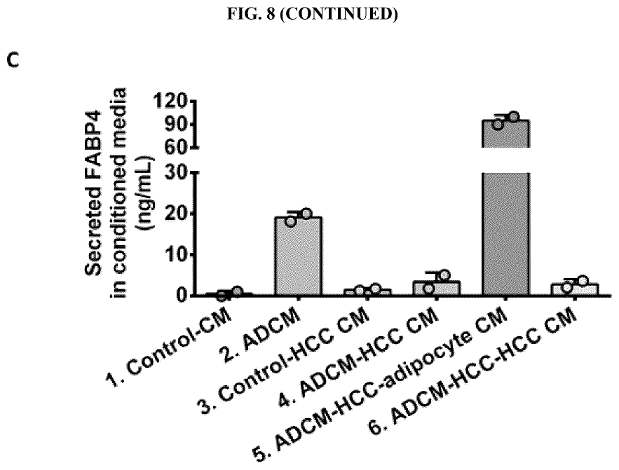

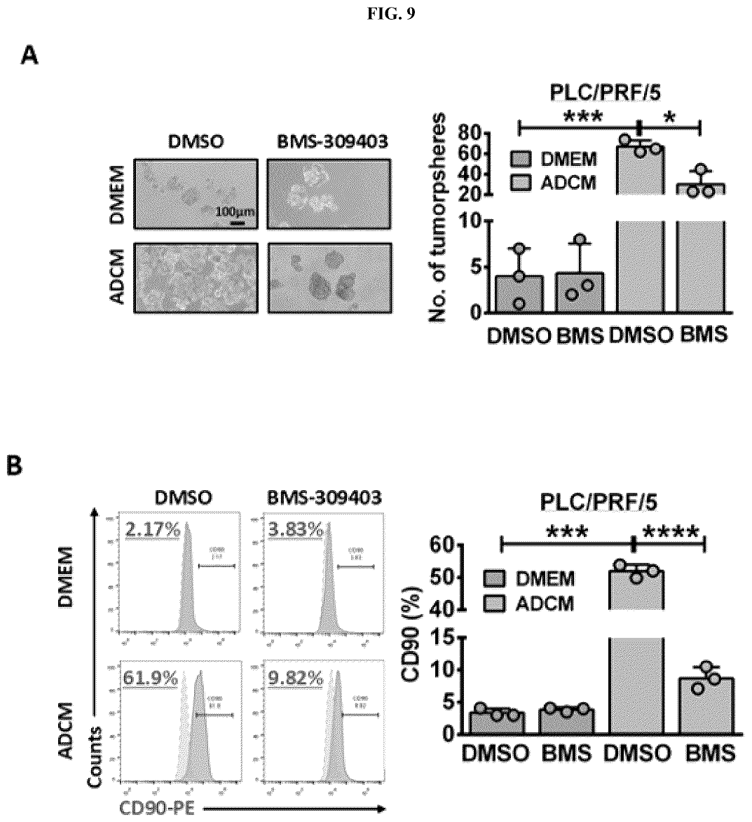

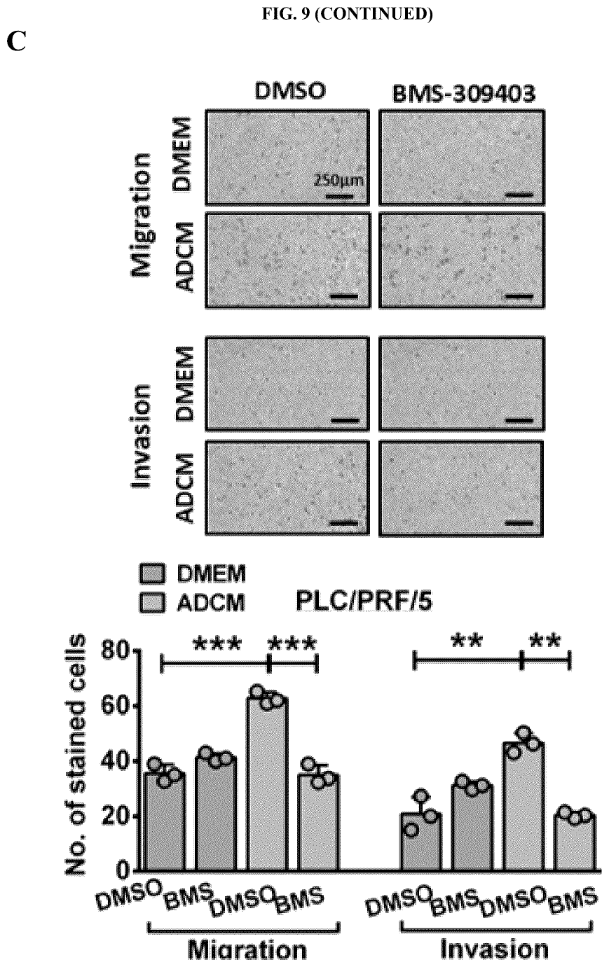

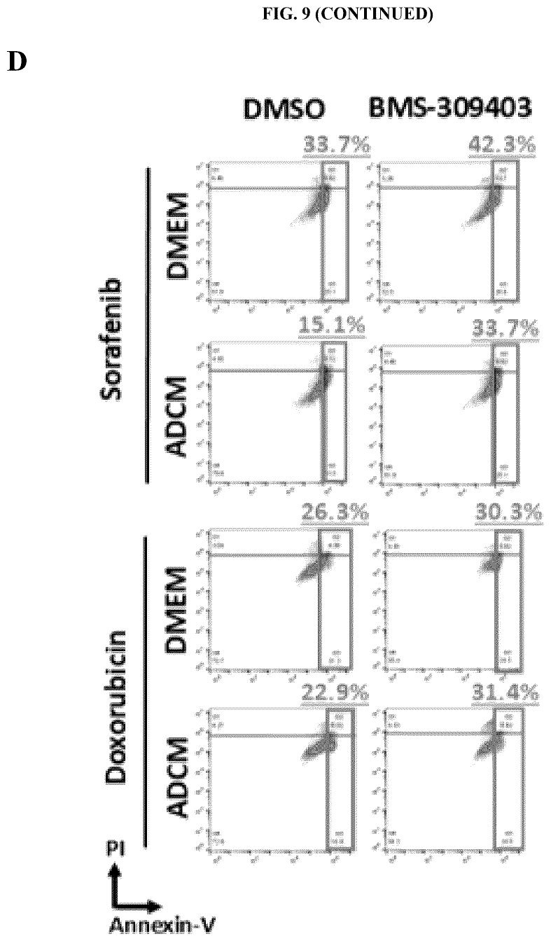

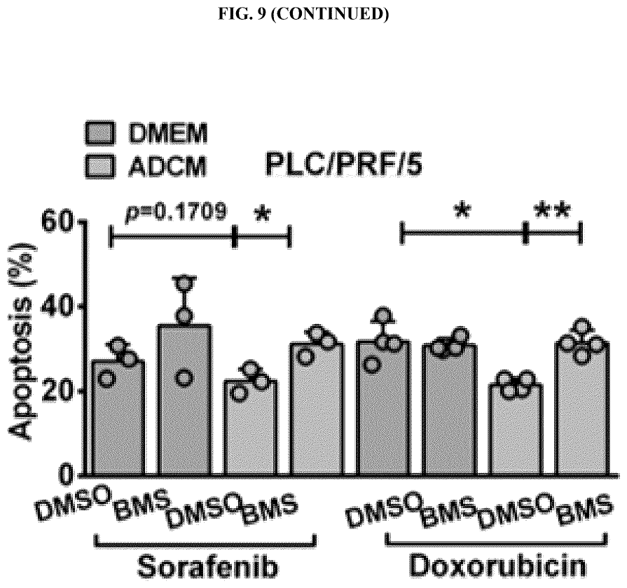

As previously described, it was found that adipocyte secretomes promote the self-renewal of hepatocellular carcinoma cells, while the cancer stem cell-enhancing effect was further enhanced by ADCM co-treatment with hepatocellular carcinoma cells (stimulated adipocyte conditioned medium, CAACM) (FIG. 8A). Based on these findings, it was intended to identify the secretory factors that drive cancer stem cell functions by identifying those factors that are not only released by adipocytes but also, most importantly, are further enhanced in co-culture with hepatocellular carcinoma cells. For this purpose, ADCM and CAACM were collected and profiled using Orbitrap mass spectrometry analysis (FIG. 2A). Using DMEM as a control, the top 22 adipocyte-specific secretory proteins were identified from ADCM (Table 2). Among these candidates, nine of them were found to be further upregulated in CAACM (FIG. 2B), identifying them as potential targets. Among the two targets (GOLM1 and FABP4) with the highest fold increase, FABP4 was selected for further functional characterization. Next, secretory FABP4 levels were compared in human visceral preadipocytes, differentiated adipocytes, hepatocellular carcinoma (HCC) cell lines, and activated human hepatic stellate cells (hTERT-HSCs). Both hepatocellular carcinoma cell lines and hTERT-HSCs produced negligible or no FABP4, similar to negative control DMEM and preadipocytes, whereas differentiated adipocytes produced detectable FABP4, indicating adipocytes as the primary FABP4 source (FIG. 2C). Furthermore, a roughly 4-fold increase in the secretory level of FABP4 (a mean of 95 ng/ml) was observed in in CAACM compared with that in ADCM, based on the conditioned media collection shown in FIGS. 8B and 8C. To investigate the functional role of adipocyte-derived FABP4 in regulation of liver cancer stem cell properties, the cancer stem cell properties of hepatocellular carcinoma cells were examined by administering recombinant human FABP4 protein (rhFABP4) at concentrations of 20 ng/ml and 100 ng/ml (mimicking physiological amounts of FABP4 in conditioned media from ADCM and CAACM). rhFABP4 was shown to promote liver cancer stem cell properties, including self-renewal (FIG. 2D), tumorigenicity (FIG. 2E and Table 3), expression of liver CSC markers (FIG. 2F), cell migration and invasion (FIG. 2G), and resistance to doxorubicin and sorafenib treatment (FIG. 2H). These data, together with the described functional observation showing attenuation of the effects of ADCM on liver cancer stem cell properties in hepatocellular carcinoma (HCC) upon treatment with a FABP4-specific inhibitor BMS-309403 (FIG. 9A to 9D), further support the role of adipocyte-derived FABP4 in the promotion of cancer stemness.

### Genetic Ablation of FABP4 Delays the Onset of Tumour Development in a MASLD-HCC Mouse Model

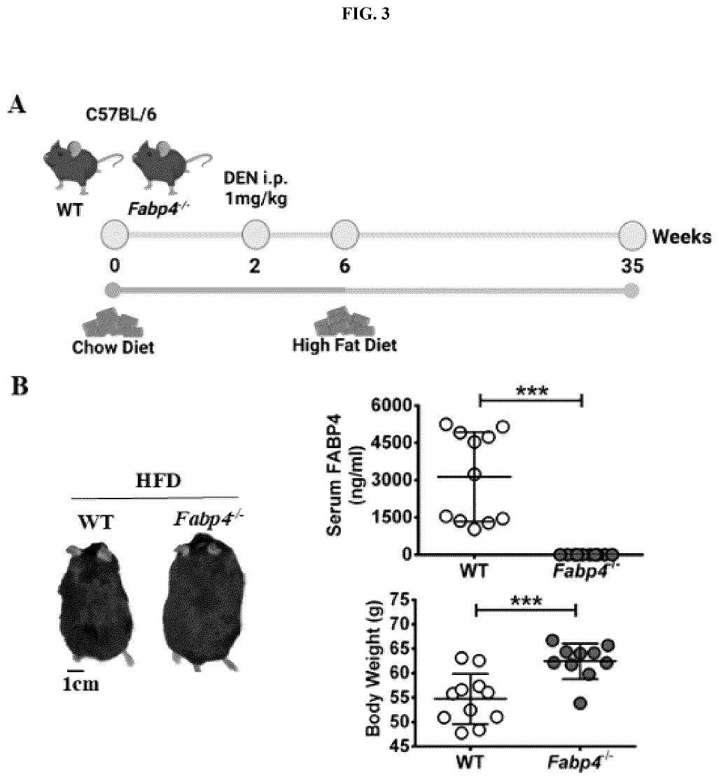

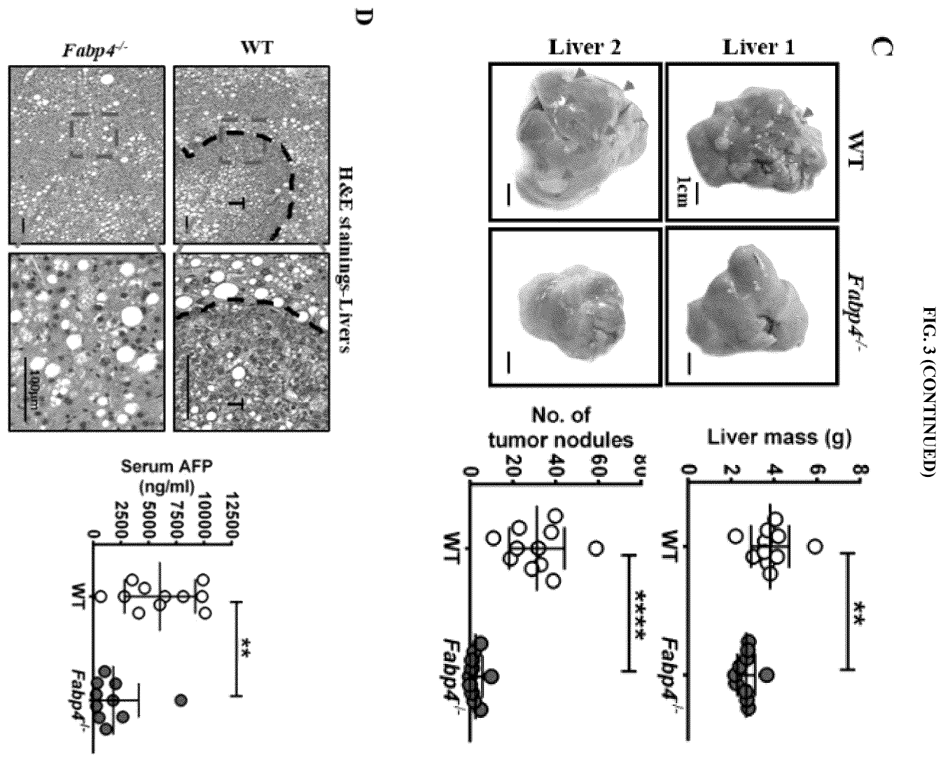

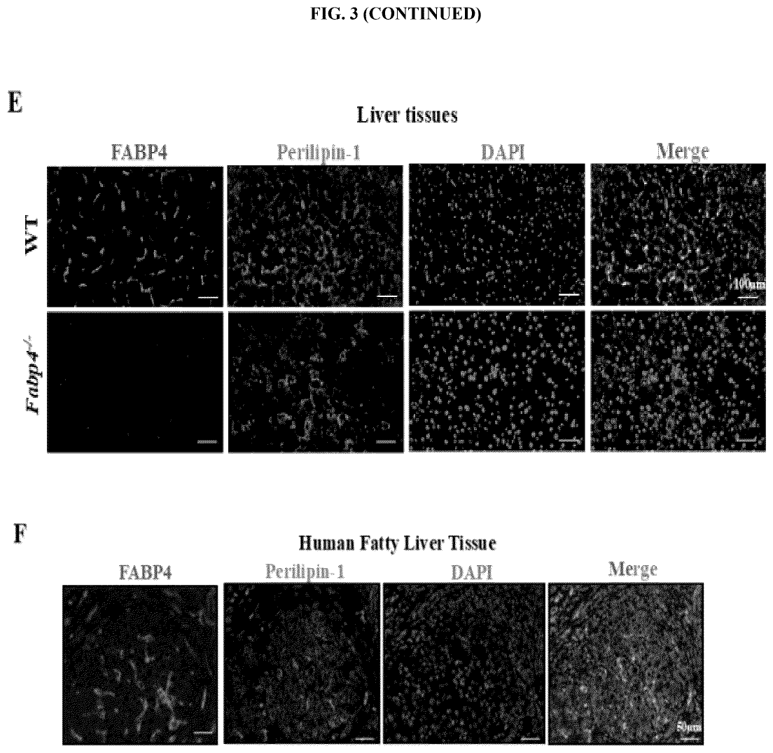

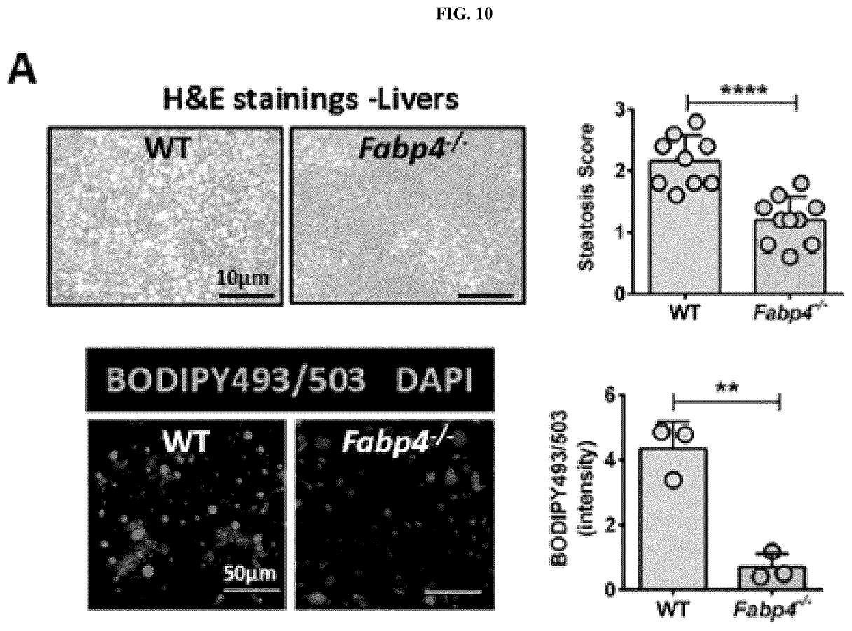

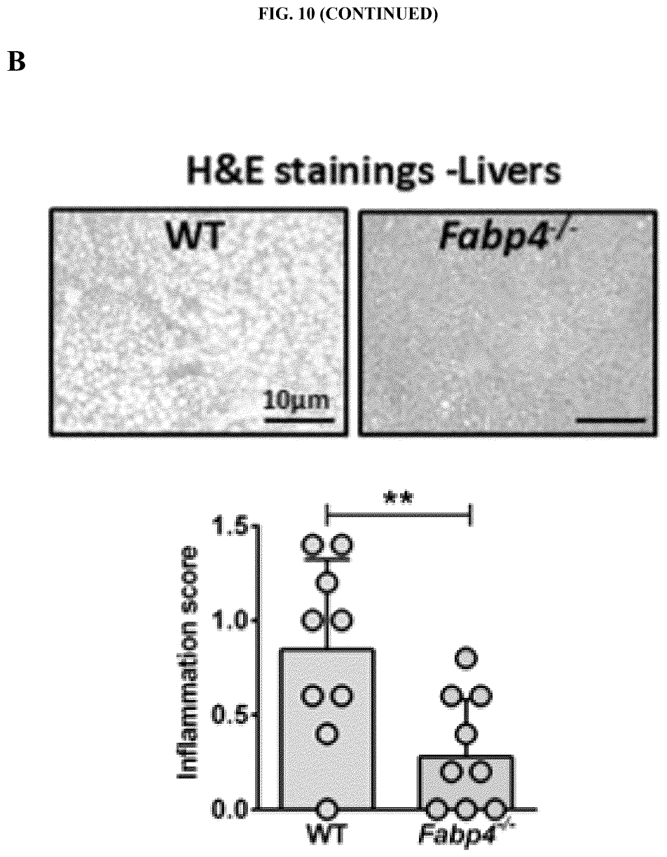

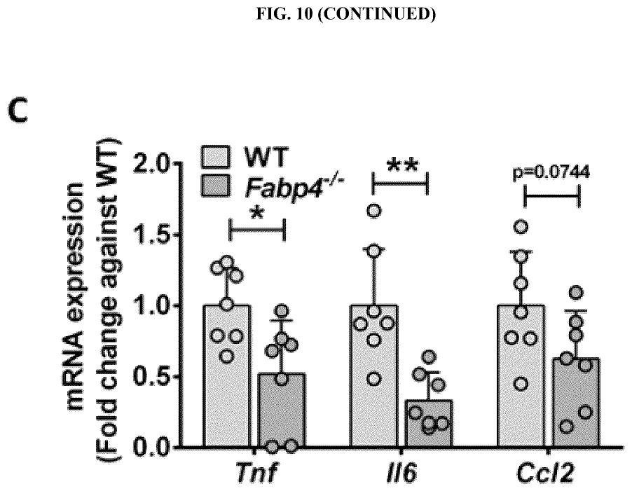

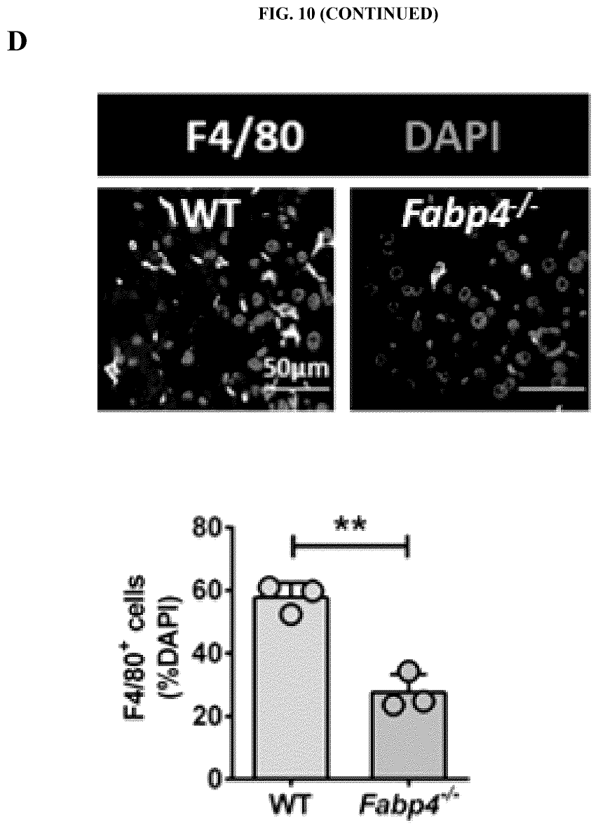

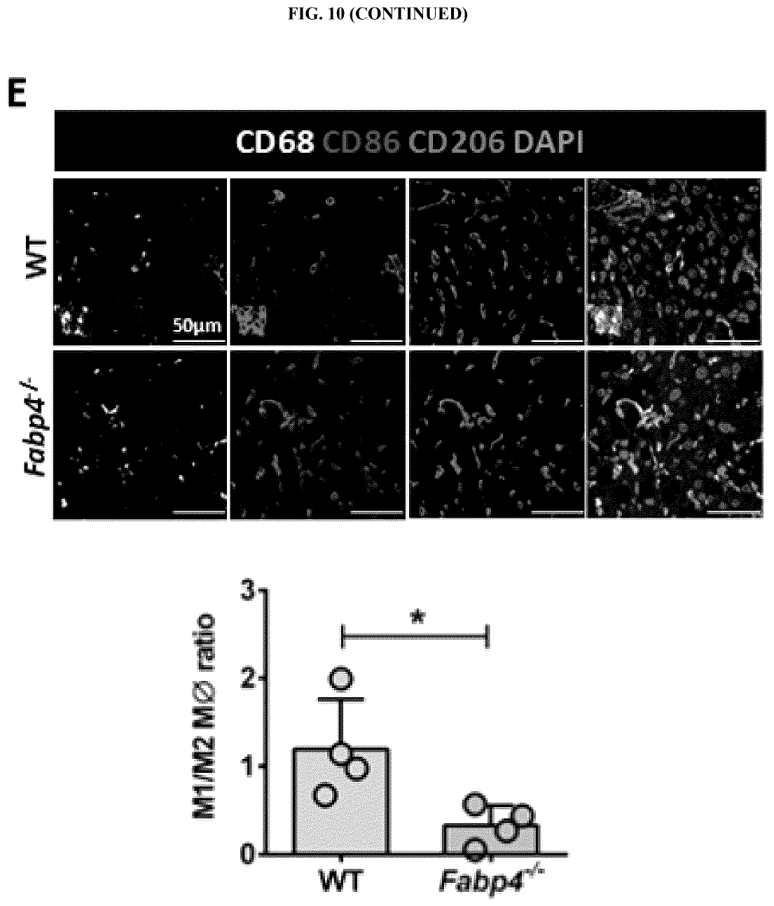

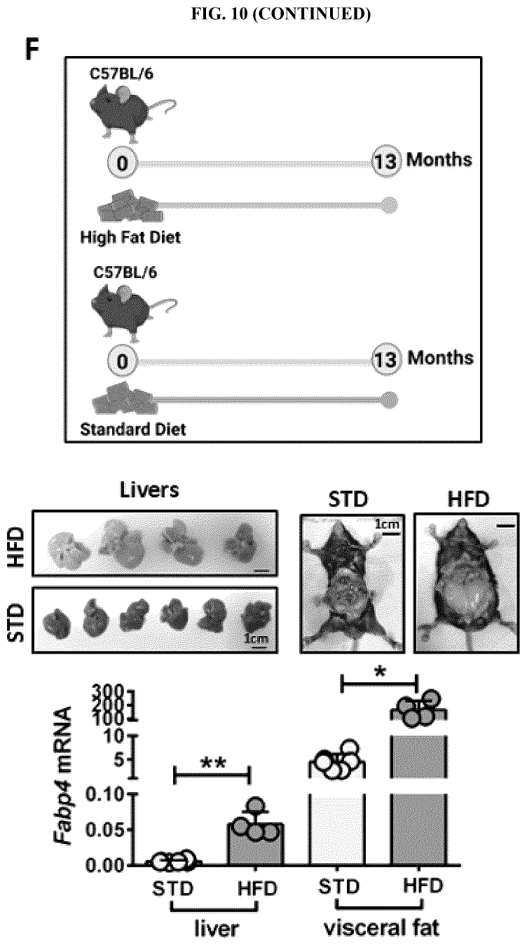

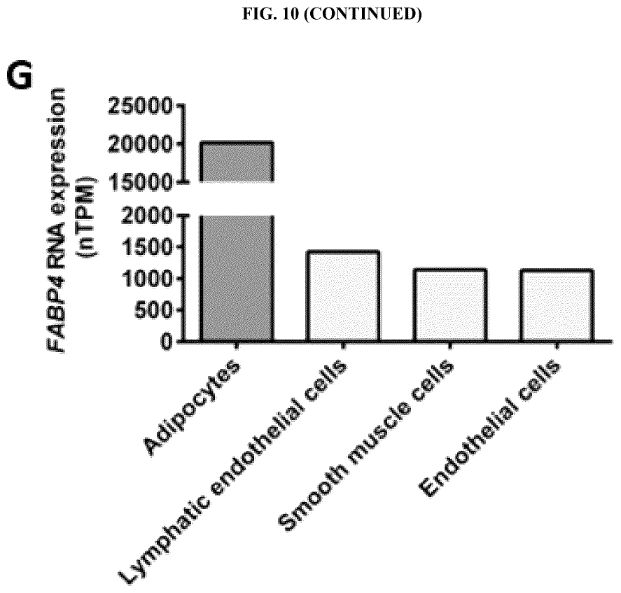

To determine the role of FABP4 in MASLD-hepatocellular carcinoma, Fabp4 knockout (KO, Fabp4^{-/-}) male mice and their wild-type (WT) littermates were subjected to diethylnitrosamine (DEN) treatment at two weeks of age and fed a high-fat diet (HFD) at six weeks of age for 29 weeks to induce MASLD-hepatocellular carcinoma formation (FIG. 3A). Secreted FABP4 was not detected in the Fabp4^{-/-} mice, and these Fabp4^{-/-} mice exhibited greater body weights than their wild-type counterparts (FIG. 3B). Without being bound by theory, impaired tumour growth was observed in Fabp4^{-/-} mice, as evidenced by a decrease in liver mass and the number of tumour nodules (FIG. 3C). The suppression of tumour growth was also shown by histological staining and a decrease in the serum alpha-fetoprotein (AFP) level (FIG. 3D). Genetic loss of FABP4 was shown to attenuate liver steatosis and inflammation, as demonstrated by steatosis and lobular inflammation scores, lipid deposition, and reduced expression of inflammatory genes, F4/80 macrophages, and M1/M2 macrophage ratio (FIG. 10A to 10E). qPCR analysis identified visceral adipocytes as the primary distant source of secreted FABP4 in mice with MASLD, contributing to the above-described liver phenotypes (FIG. 10F). This is in alignment with protein atlas single-cell data showing adipocytes having the highest FABP4 expression among cell types (FIG. 10G). Furthermore, colocalization of FABP4 with perilipin-1 demonstrated the preferential expression of FABP4 in lipid droplets in steatotic hepatocytes (FIG. 3E). The specificity of FABP4 in lipid droplet was also evident in human fatty liver tissue (FIG. 3F). These findings indicate that visceral adipocytes and lipid droplet-rich in steatotic hepatocytes are sources of secretory FABP4 in MASLD-induced mice.

### FABP4 is Sporadically Expressed in MASLD-Induced Hepatocellular Carcinoma Patients With Clinical Significance

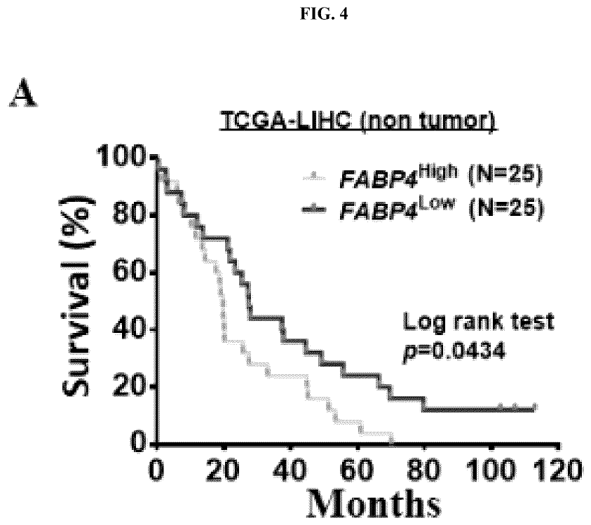

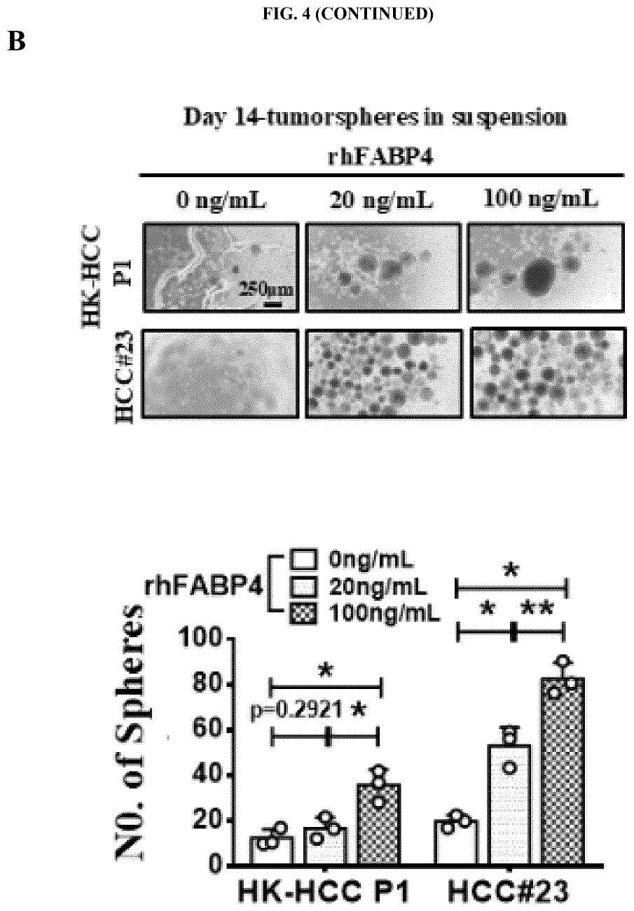

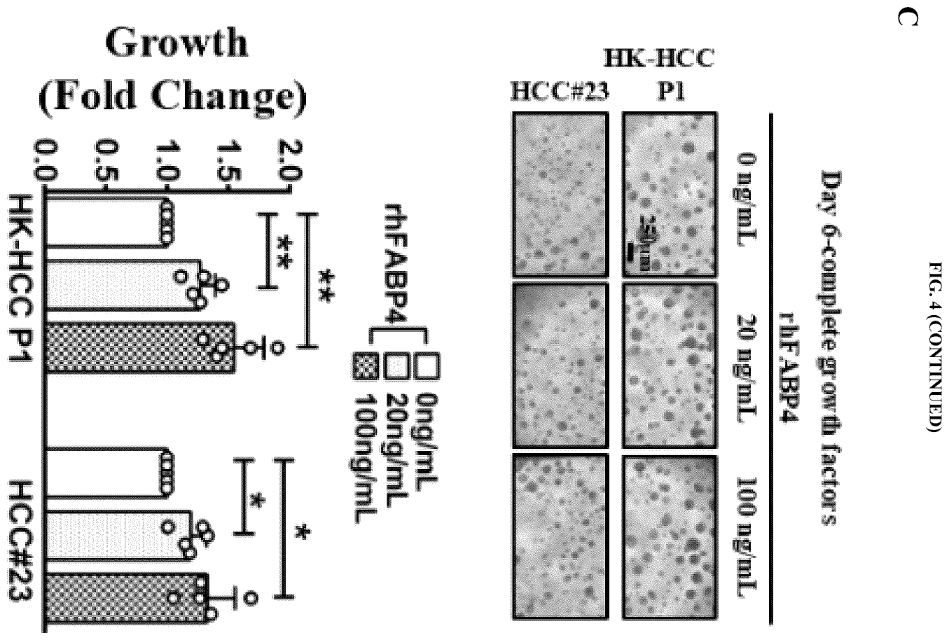

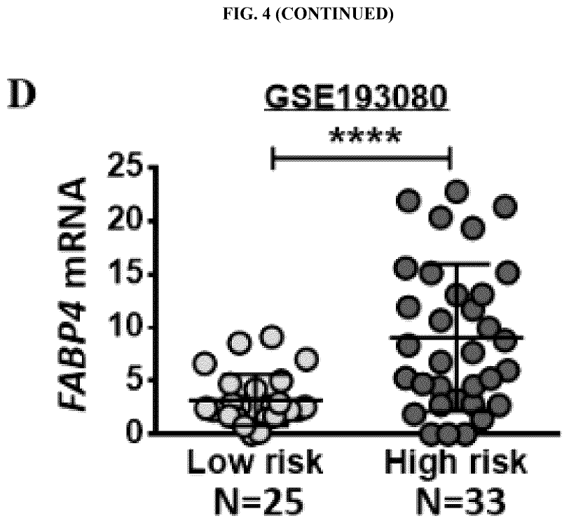

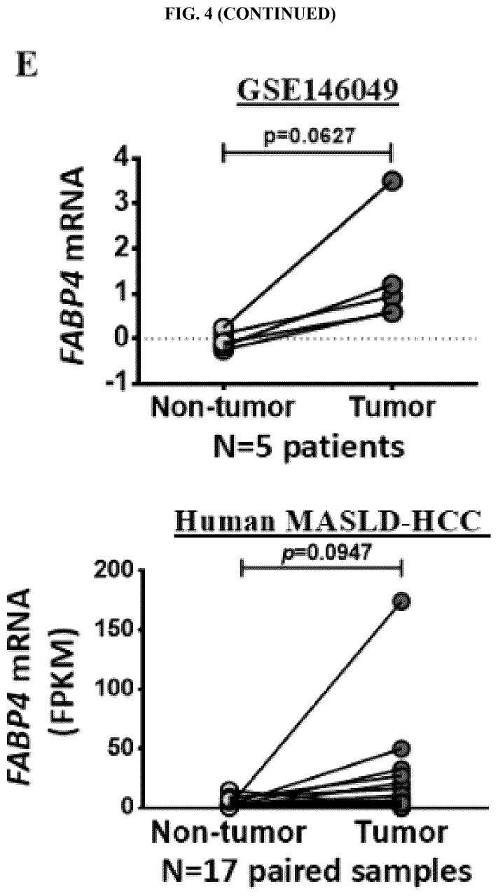

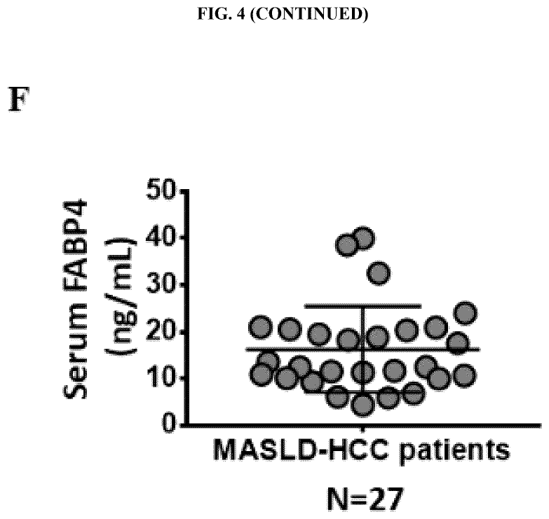

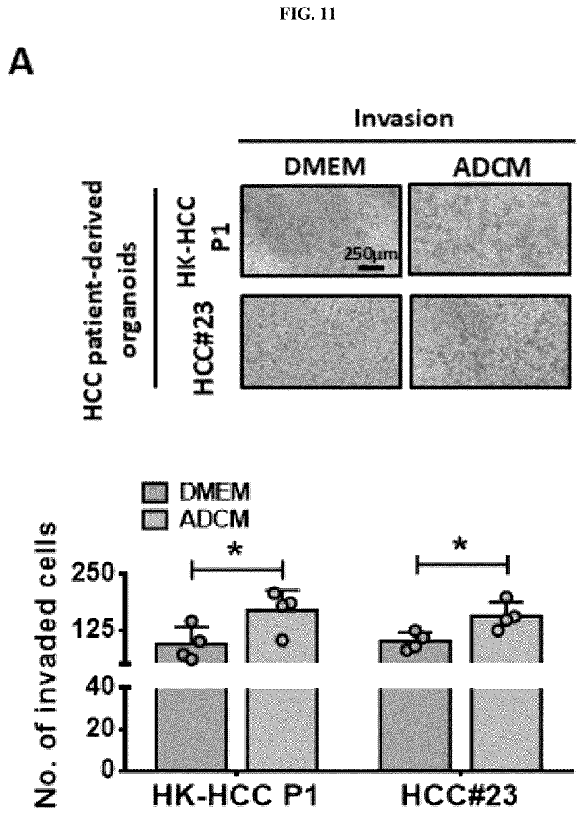

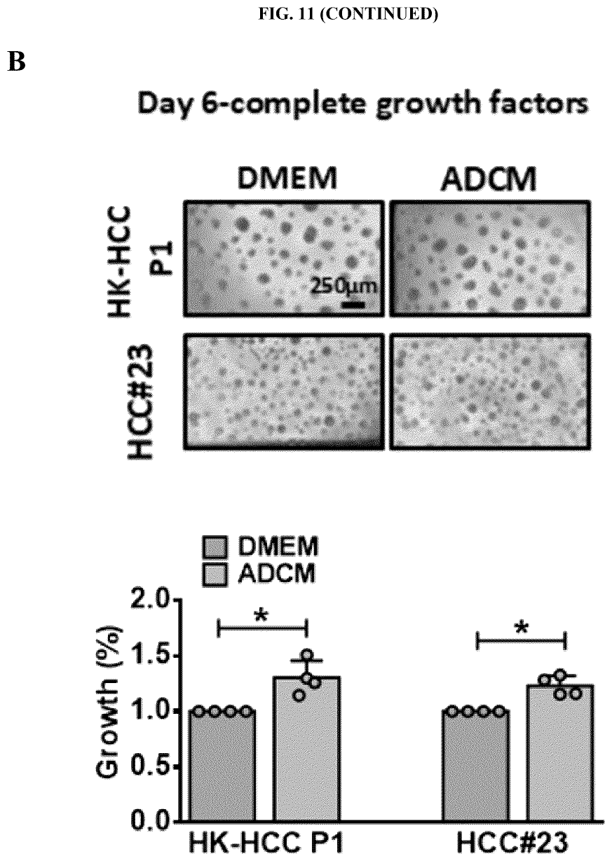

Analysis of the non-tumorous samples from The Cancer Genome Atlas Liver Hepatocellular Carcinoma cohort revealed that patients with high FABP4 level had a shorter overall survival (FIG. 4A). The effect of rhFABP4 was further analysed in a more clinically relevant setting of an organotypic ex vivo culture of primary hepatocellular carcinoma tumours, i.e., hepatocellular carcinoma (HCC) patient-derived organoids (HK-HCC P1 and HCC #23). rhFABP4 was shown to confer self-renewal and was shown to increase the size of organoids and their proliferative rate (FIG. 4B, 4C). Likewise, the secretome in ADCM was shown to increase the invasiveness and proliferation rate of hepatocellular carcinoma organoids (FIG. 11A, 11B). MASLD patients exhibiting high FABP4 expression were shown to be at an elevated risk for developing hepatocellular carcinomas (FIG. 4D). Next, the expression level of FABP4 was examined in MASLD-hepatocellular carcinoma patients. FABP4 was shown to be upregulated in MASLD-induced hepatocellular carcinoma clinical samples compared with their adjacent, normal counterparts in publicly available datasets and an in-house cohort (FIG. 4E). Overexpression of FABP4 in these patients in different cohorts showed a trend of hepatocellular carcinoma recurrence (GSE214432, p=0.0994, data not shown). Finally, the secretory level of FABP4 in MASLD-induced hepatocellular carcinoma patients was assessed to determine whether the doses used in the studies disclosed herein were physiologically relevant. A mean serum FABP4 level of 16.24 ng/ml was determined, which corresponds to the physiological dose applied in in vitro and in vivo experiments (FIG. 4F) described herein. Moreover, administering rhFABP4 at a concentration of 40 ng/ml, which matches the peak FABP4 level observed in patients with MASLD-induced hepatocellular carcinoma, was shown to have an augmenting effect on liver cancer stem cell characteristics (FIG. 2D to 2H).

Thus, in one example, there is disclosed a method of treating hepatocellular carcinoma in a subject, comprising administering an effective amount of a monoclonal antibody to the subject, wherein the monoclonal antibody specifically binds to fatty acid binding protein 4 (FABP4).

In one example, the antibody used in the method disclosed herein is a human, humanised, or murine antibody. In another example, the antibody is a human, or humanised antibody.

In another example, administration of the effective amount of the monoclonal antibody to the subject in accordance with the method disclosed herein results in one or more of the following: reduction in cell migration of hepatocellular carcinoma cells; reduction in cell invasiveness of hepatocellular carcinoma cells; reduction in self-renewal of hepatocellular carcinoma cells; and reduction in cancer stemness of hepatocellular carcinoma cells. In one example, the reduction is in comparison to the same feature as presented in a hepatocellular carcinoma cell prior to treatment.

In one example, the disease to be treated is hepatocellular carcinoma. In another example, hepatocellular carcinoma is metabolic dysfunction-associated steatotic liver disease-related hepatocellular carcinoma (MASLD-HCC). In yet another example, the hepatocellular carcinoma is a result of metabolic-associated steatohepatitis (MASH).

### FABP4 Activates the Wnt/β-Catenin Pathway via the Phosphorylation of AKT

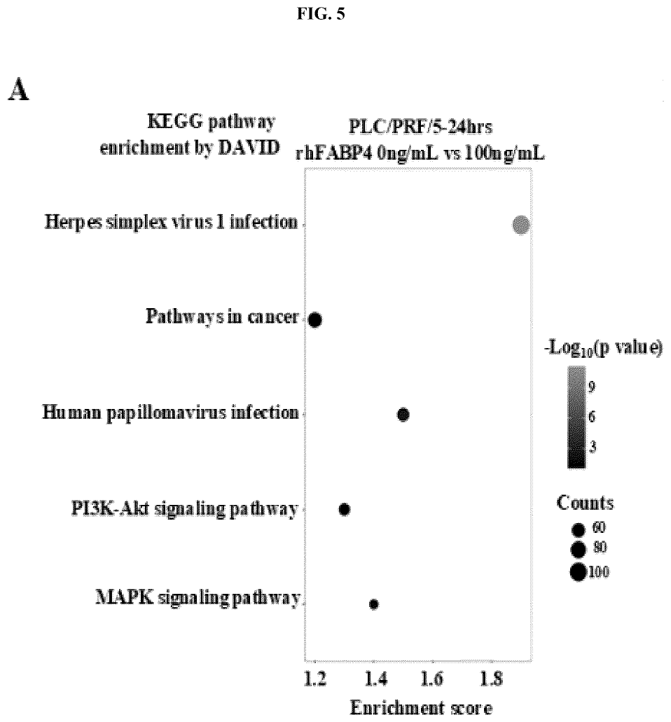

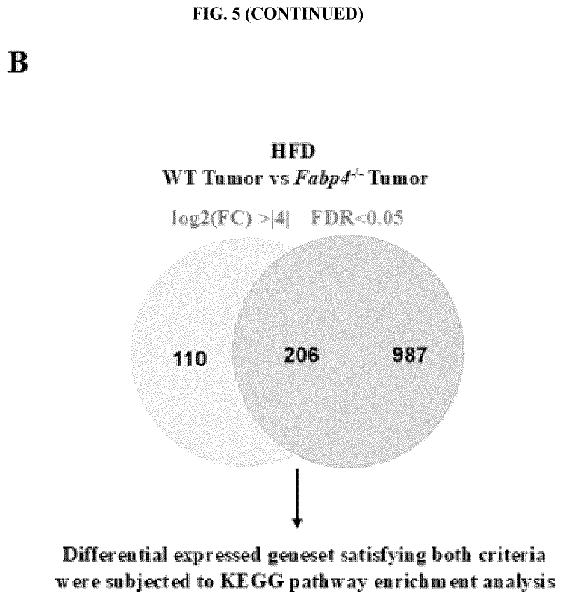

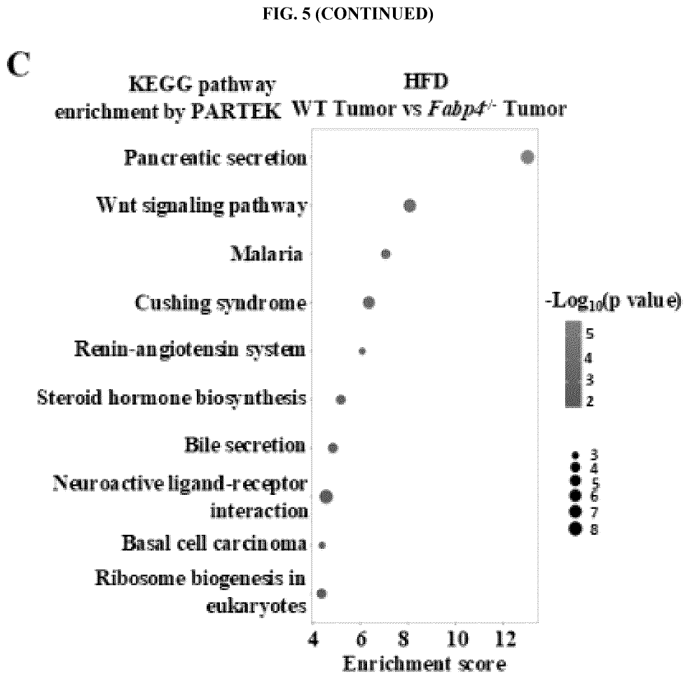

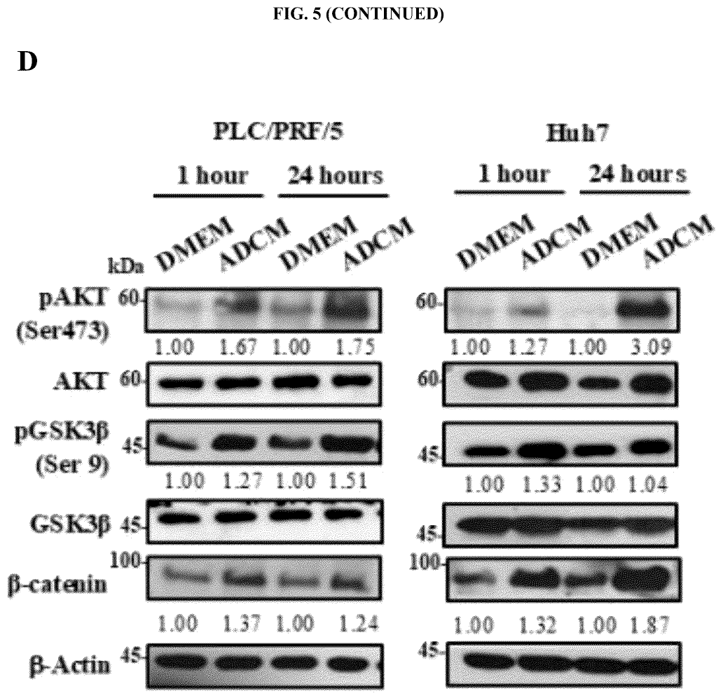

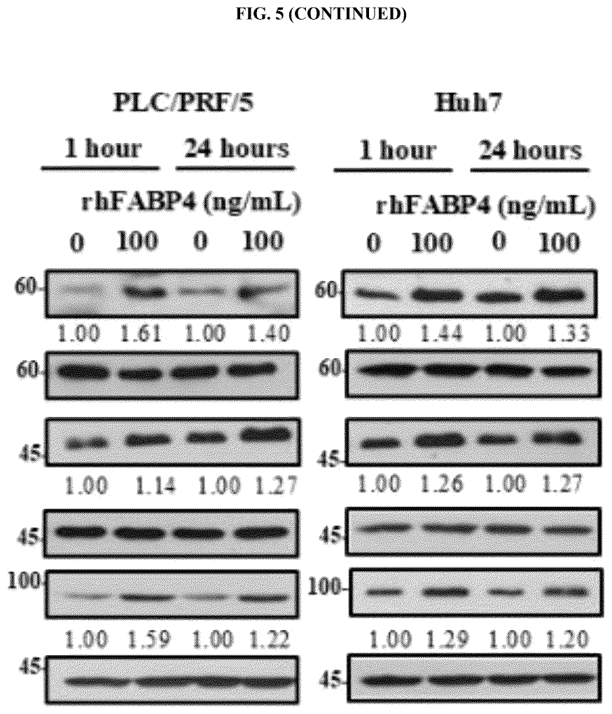

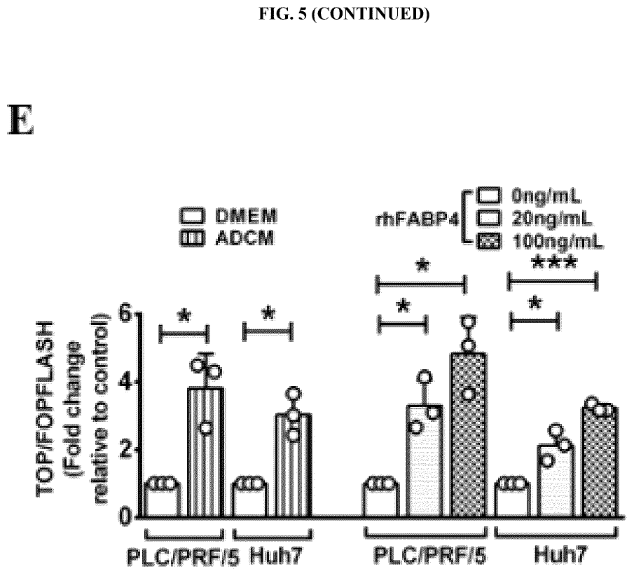

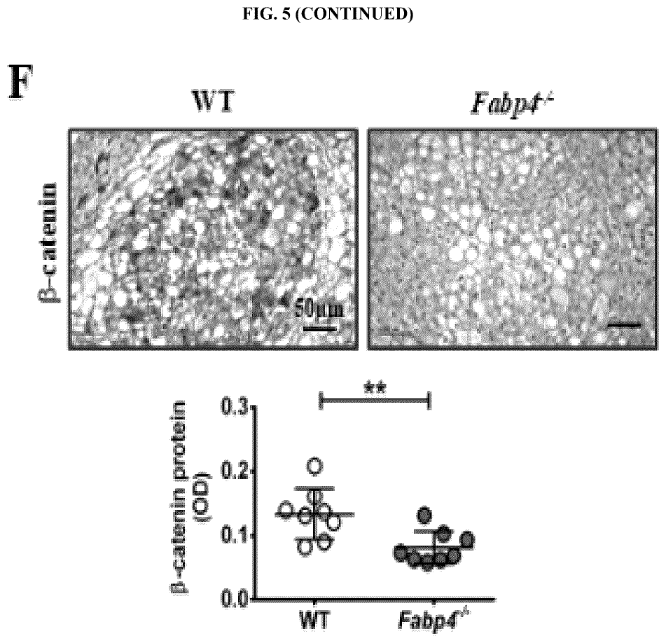

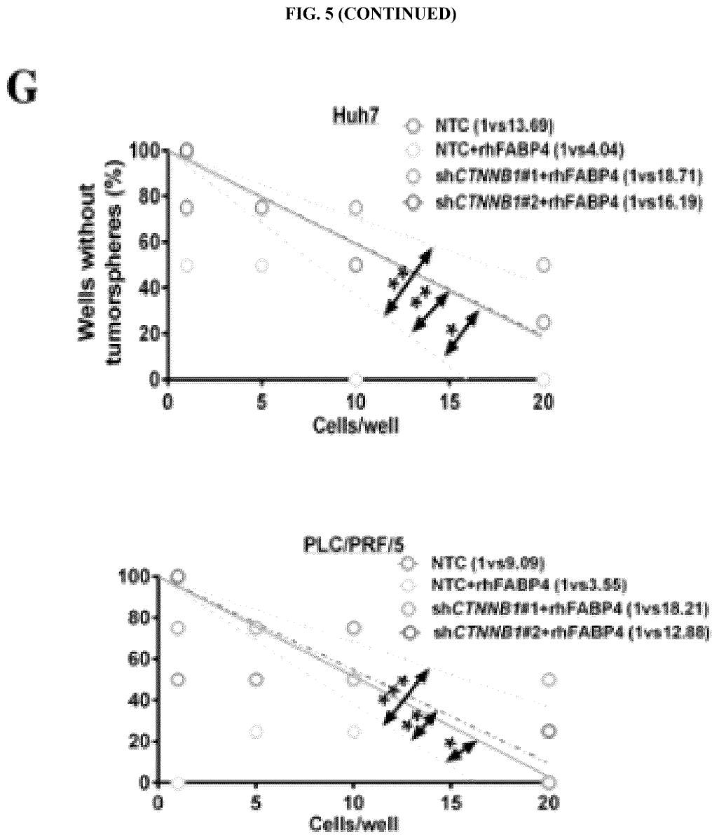

To elucidate the mechanisms by which FABP4 regulates cancer stemness of hepatocellular carcinoma cells, bulk RNA sequencing profiling was performed using PLC/PRF/5 cells that had either been pretreated with 0 ng/mL or 100 ng/mL rhFABP4 for 24 hours. Kyoto Encyclopedia of Genes and Genomes (KEGG) pathway enrichment analysis using the Database for Annotation, Visualization and Integrated Discovery (DAVID) showed that PI3K/AKT signalling was enriched upon treatment of rhFABP4 (FIG. 5A). Furthermore, the enriched signalling pathways were analysed in tumours harvested from wild-type (WT) and Fabp4^{-/-} mice after HFD feeding based on KEGG pathway enrichment analysis using Partek Genomics Suite (FIG. 5B). Among the top ten enriched pathways, the Wnt signalling pathway was the only stemness-related pathway shown to be enriched in wild-type tumours (FIG. 5C). It was also shown that Ctnnb1 was downregulated in Fabp4^{-/-} tumours (data not shown). Using Western blot analysis, it was found that AKT was consistently activated through phosphorylation at Ser473 upon ADCM and rhFABP4 treatment, which in turn lead to the inactivation of GSK3β via phosphorylation at Ser9, further resulting in the accumulation of total β-catenin (FIG. 5D). The transactivation of β-catenin was also shown to be enhanced, as determined by the TOP/FOP reporter assay (FIG. 5E). The protein level of β-catenin was shown to be lower in the liver tumours of Fabp4^{-/-} mice (FIG. 5F). Lastly, involvement of β-catenin in FABP4-mediated cancer stem cell function was confirmed by suppressing β-catenin in rhFABP4-treated hepatocellular carcinoma cells (FIG. 5G). The above results indicate that exogenous FABP4 regulates Wnt/β-catenin signalling through activating PI3K/AKT in hepatocellular carcinoma cells.

### Exogenous FABP4 Directly Binds to the Membrane Receptor ITGB1, Driving the PI3K/AKT/β-Catenin Signalling Cascade

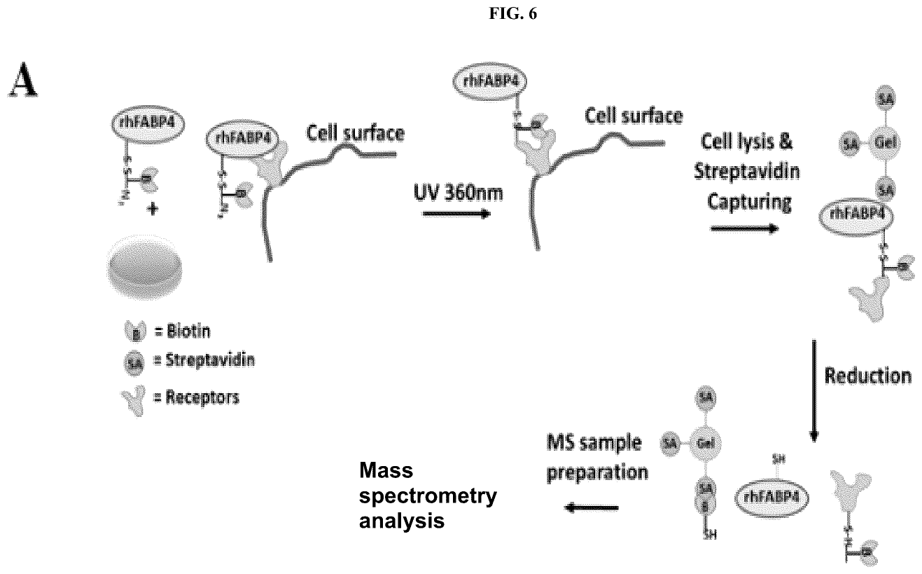

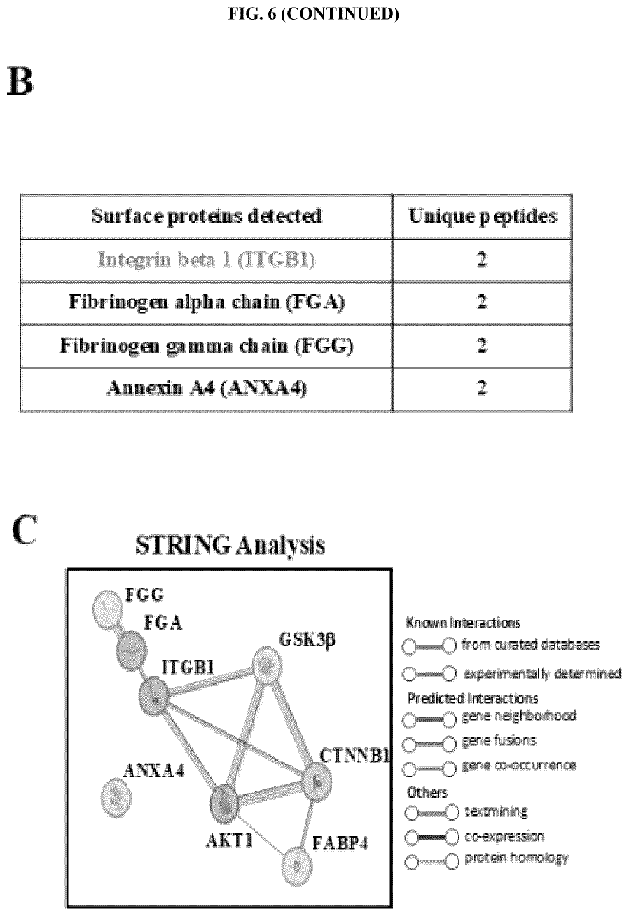

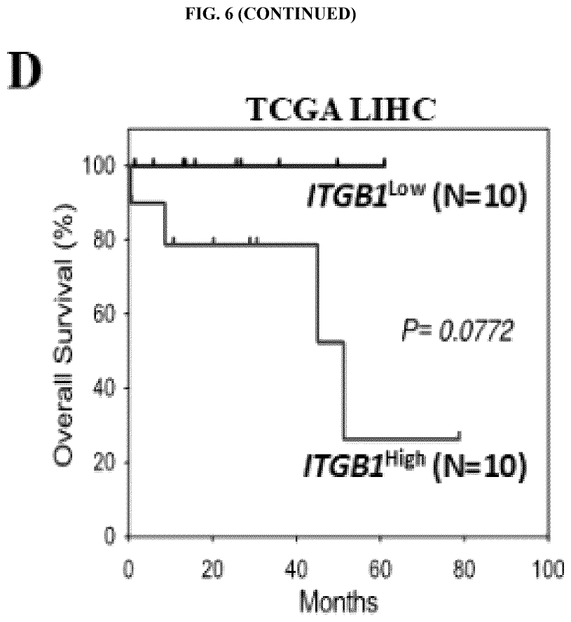

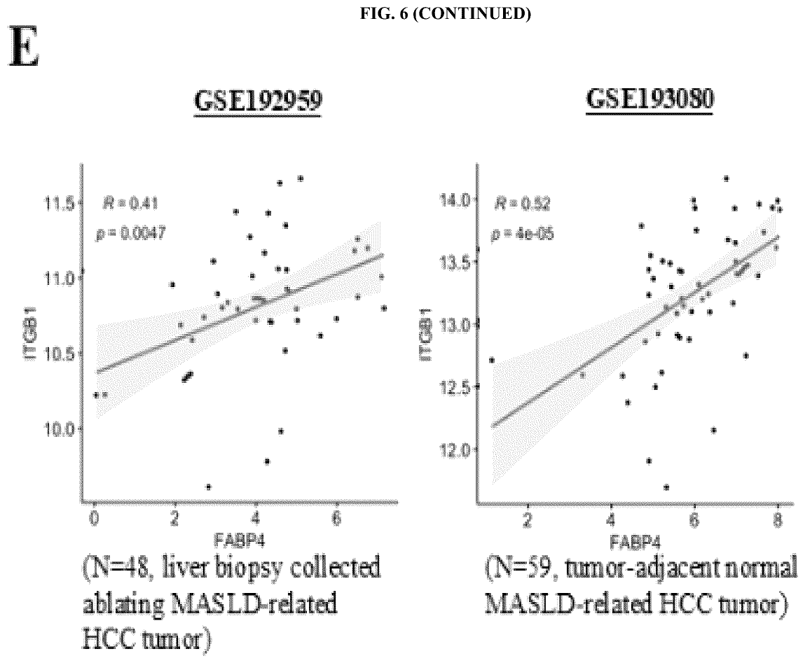

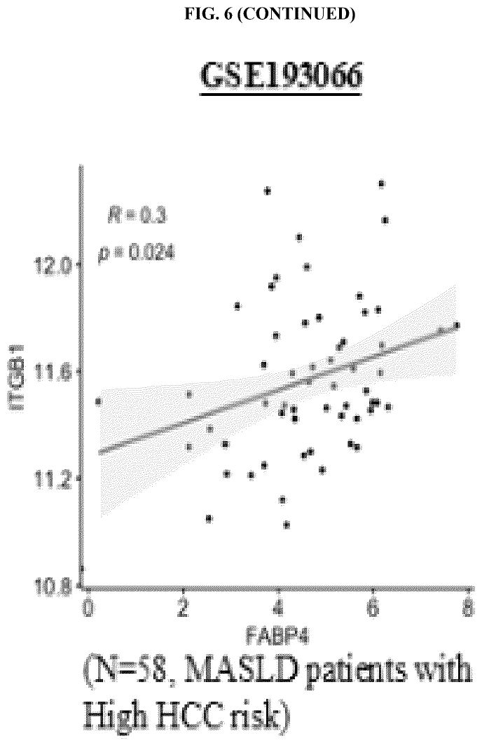

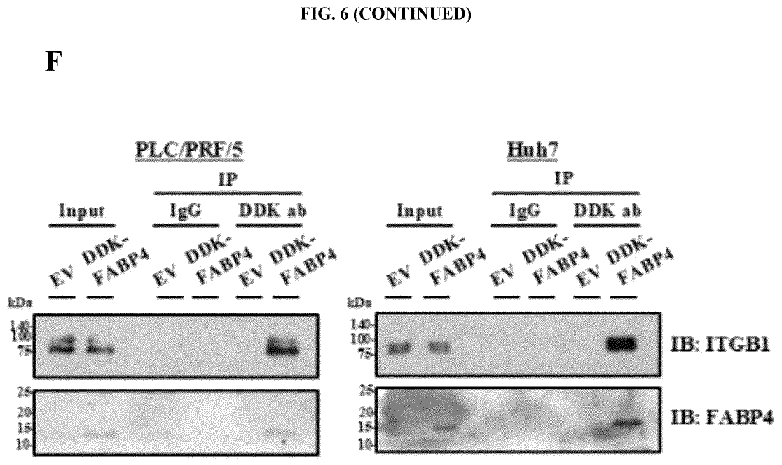

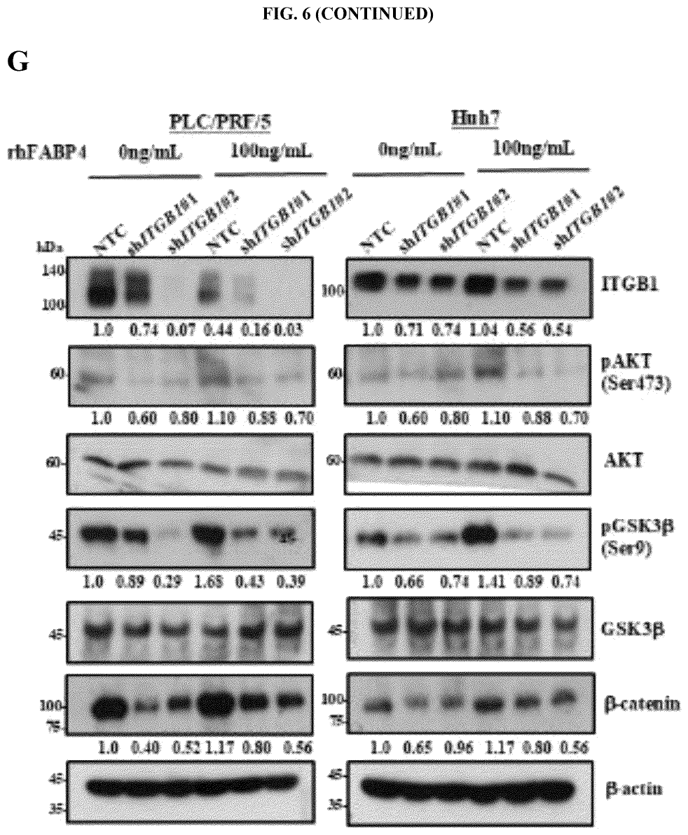

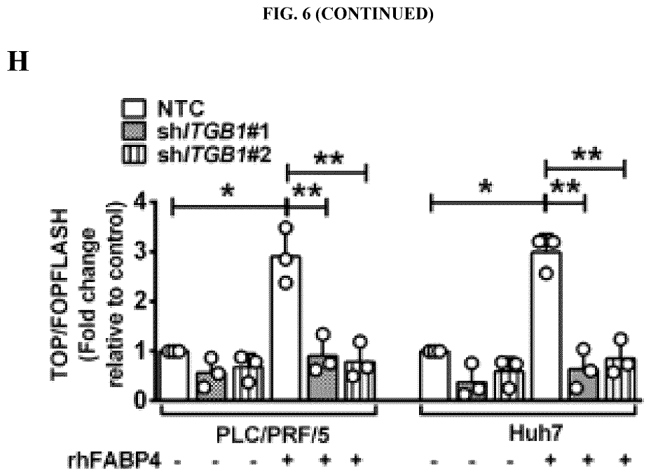

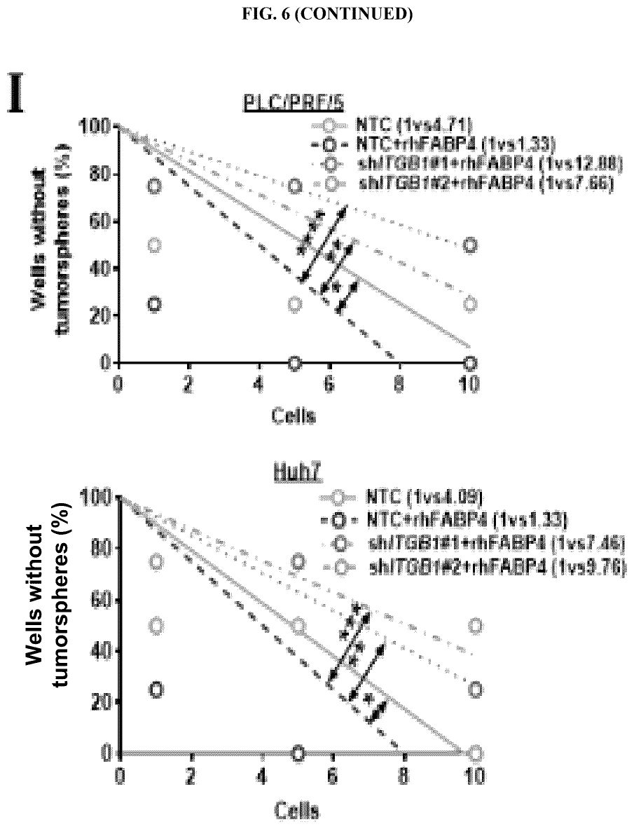

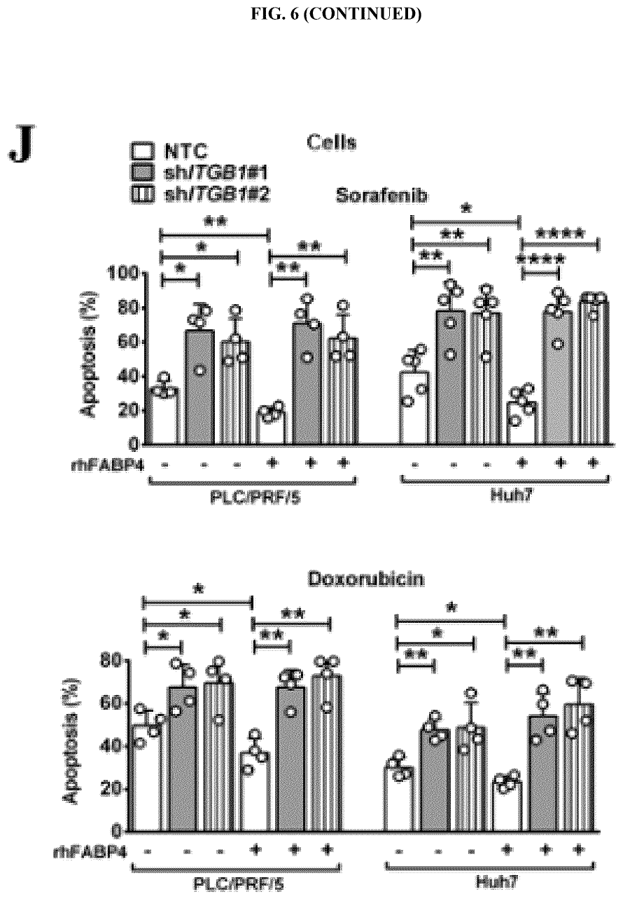

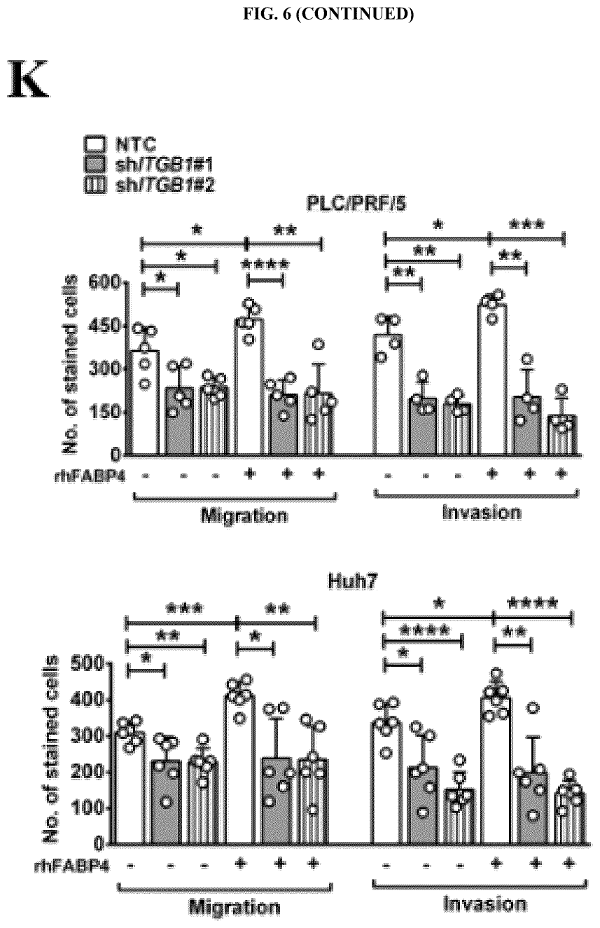

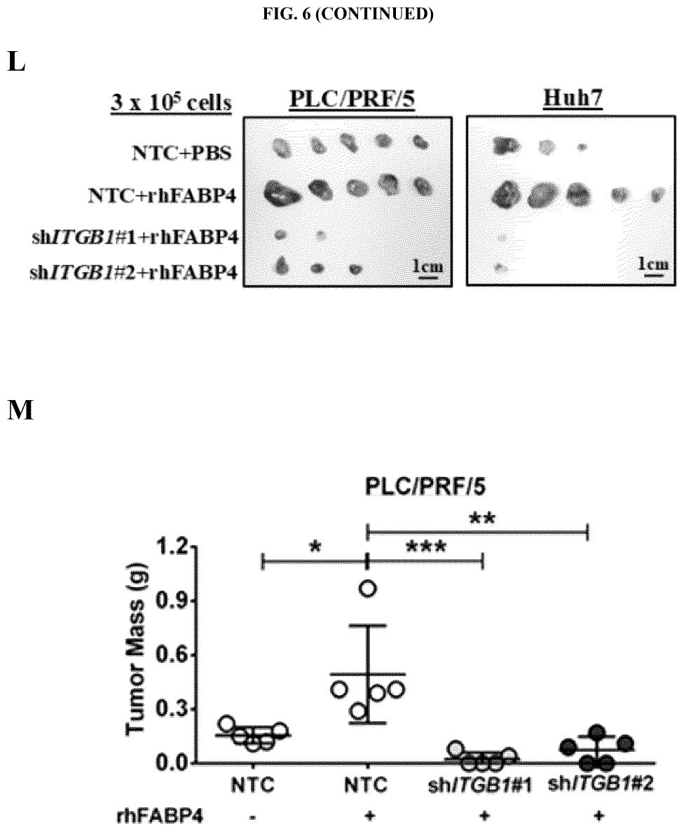

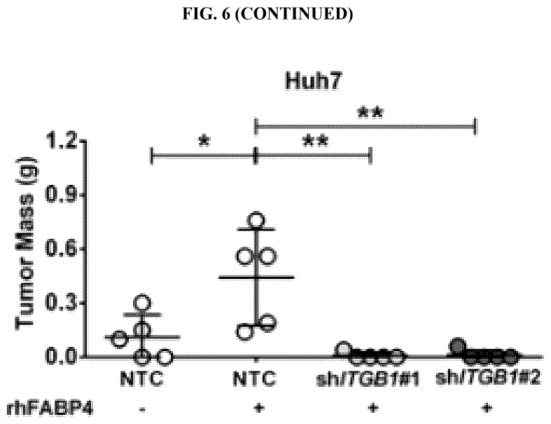

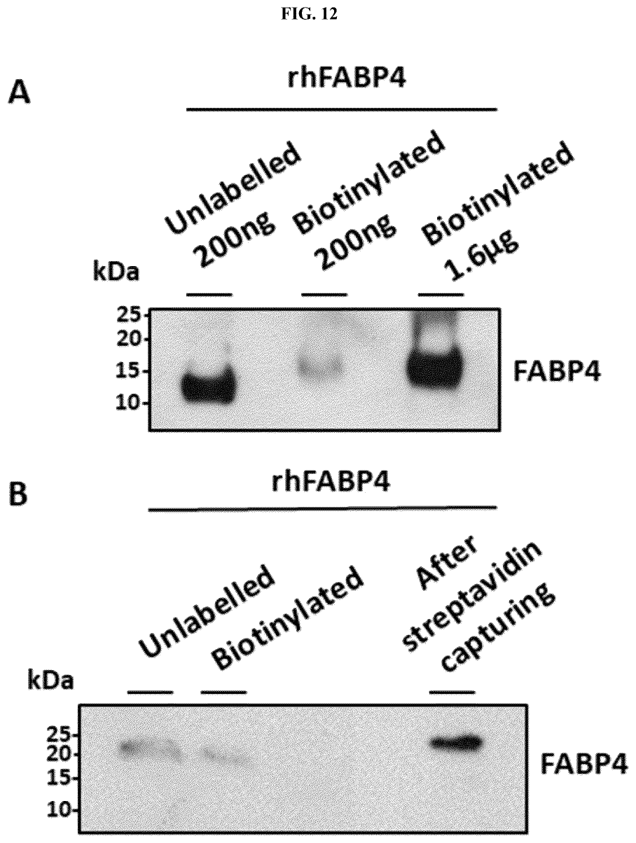

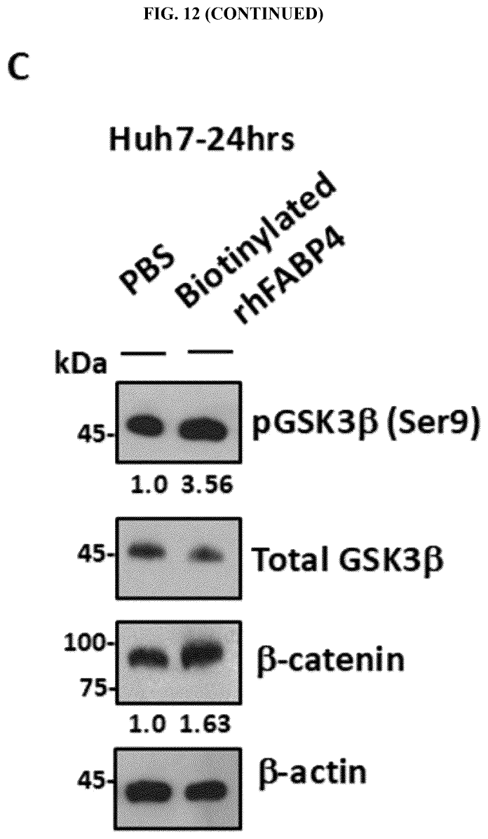

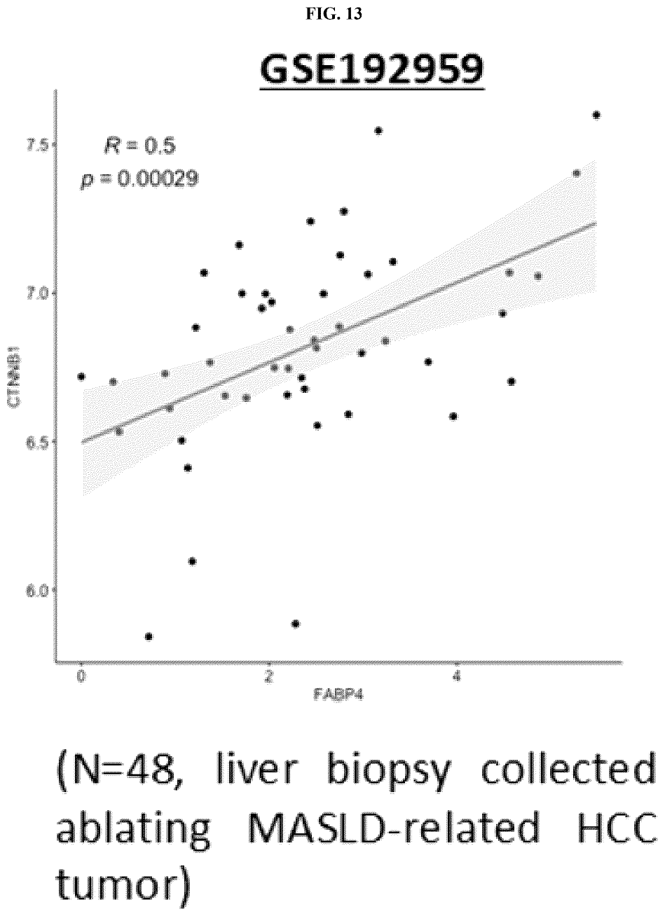

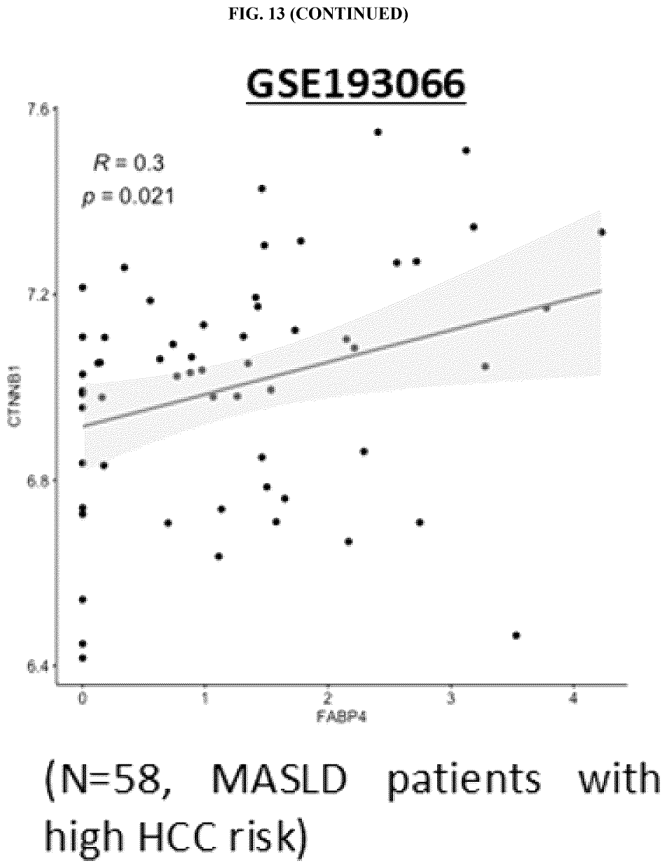

Mass spectrometry analysis was used to identify potential FABP4-binding receptors on the membrane surface of Huh7 cells using biotinylated rhFAPB4 (FIG. 6A and FIG. 12). In total, four surface proteins were detected (FIG. 6B). These surface proteins, together with FABP4 and the main canonical components controlling Wnt/β-catenin signalling, were subjected to STRING analysis. It is known that ITGB1 potentially interacts with AKT1, GSK3β and CTNNB1 (β-catenin) (FIG. 6C), indicating a possible role that ITGB1 plays in the regulatory circuit through which FABP4 mediates the activation of Wnt/β-catenin signalling in hepatocellular carcinoma. Clinically, high ITGB1 expression in hepatocellular carcinoma patients with MASLD background shows a trend of shorter overall survival (FIG. 6D). Furthermore, FABP4 expression was shown herein to positively correlated with ITGB1 and CTNNB1 in MASLD-related hepatocellular carcinoma patients and MASLD patients at high risk of hepatocellular carcinoma (FIG. 6E and FIG. 13). Consistent with the mass spectrometry findings, immunoprecipitation data showed a physical interaction between FABP4 and ITGB1 (FIG. 6F). Stable knockdown of ITGB1 in hepatocellular carcinoma cells leads to downregulation of p-AKT (Ser473) and p-GSK3β(Ser9), resulting in a reduction in total β-catenin accumulation and transactivation (FIG. 6G, 6H). The effects of exogenous FABP4 were shown to be eliminated by the repression of ITGB1 (FIG. 6G, 6H). Furthermore, it was seen that the enhancing effects of rhFABP4 on self-renewal ability, drug resistance and migration and invasion capabilities were offset upon the repression of ITGB1 (FIGS. 6I to K). In vivo observations also showed that suppression of ITGB1 mitigated the effect of rhFABP4-induced hepatocellular carcinoma tumour growth (FIG. 6L, 6M). Collectively, this data shows that ITGB1 is a membrane receptor that mediates cancer stemness and drug resistance in hepatocellular carcinoma cells via the PI3K/AKT/β-catenin signalling cascade.

### Anti-FABP4 Neutralising Monoclonal Antibody Suppresses Cancer Stemness and Inhibits Tumour Growth in a MASLD-Hepatocellular Carcinoma (HCC) Mouse Model

A monoclonal antibody targeting FABP4 (anti-FABP4 mAb, 3I19-1) was developed and its therapeutic potential evaluated for treating MASLD related hepatocellular carcinoma.

As used herein, the term “antibody” is used in the broadest sense and specifically covers full length monoclonal antibodies, polyclonal antibodies, and antibody fragments, so long as they exhibit the desired biological activity.

Thus, in one example, there is disclosed a monoclonal antibody which binds to fatty acid binding protein 4 (FABP4).

Antibodies to a given target antigen can be made, derived or engineered using techniques known in the art. Such techniques include, but are not limited to, screening antibody gene-phage display libraries for molecules capable of binding to the target antigen, and raising antibodies to a given target antigen by animal immunisation. Approaches to the production of monoclonal antibodies (monoclonal or otherwise) suitable for therapeutic use in humans, or for use according to the methods disclosed herein, include, but are not limited to, using hybridoma technology, raising xenogeneic antibodies and subsequent humanisation, human antibody gene-phage display, and production in transgenic mice having human antibody genes.

In another example, the monoclonal antibody binds to a sequence of FABP4 comprising or consisting of SEQ ID NO. 1.

As used herein, the term “monoclonal antibody” refers to an antibody obtained from a population of substantially homogeneous antibodies, that is to say, the individual antibodies comprising the population are identical and/or bind the same epitope, except for possible variants that may arise during production of the monoclonal antibody, such variants generally being present in minor amounts. In contrast to polyclonal antibody preparations, which can typically include different antibodies directed against different determinants (epitopes), each monoclonal antibody is directed against a single determinant (epitope) on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method.

In one example, the antibody disclosed herein is obtained from a hybridoma cell line.

Antibodies to a given target, target protein or protein complex raised in a non-human animal (for example, but not limited to, mouse, rabbit, horse, dog, donkey, or any other suitable animal), can be engineered in order to improve their suitability for therapeutic use in humans, also known as humanisation or a humanised antibody. In other words, humanised antibodies are antibodies whose protein sequences have been modified to increase their similarity to antibody variants produced naturally in humans. For example, one or more amino acids of monoclonal antibodies raised by animal immunisation can be substituted to arrive at an antibody sequence which is more similar to human germline immunoglobulin sequences, thereby reducing the potential for an (anti-xenogeneic) antibody immune responses in a human subject treated with the antibody. Modifications in the antibody variable domains can focus on the framework regions in order to preserve the antibody paratope. The requirement for humanisation can be circumvented by, for example, raising antibodies to a given target protein/protein complex in transgenic model species expressing human immunoglobulin genes, such that the antibodies raised in such animals are fully human.

Thus, in one example, the antibody disclosed herein is a human, humanised, or murine antibody. In another example, the antibody disclosed herein is a murine antibody. In another example, the antibody disclosed herein is a humanised antibody.

Depending on the amino acid sequence of the constant domain of their heavy chains, full length antibodies can be assigned to different “classes”. There are five major classes of full-length antibodies: IgA, IgD, IgE, IgG, and IgM, and several of these may be further divided into “subclasses” (isotypes), e.g., IgG1, IgG2, IgG3, IgG4, IgA, and IgA2.

In one example, the antibody is an IgG, IgA, or IgM antibody.

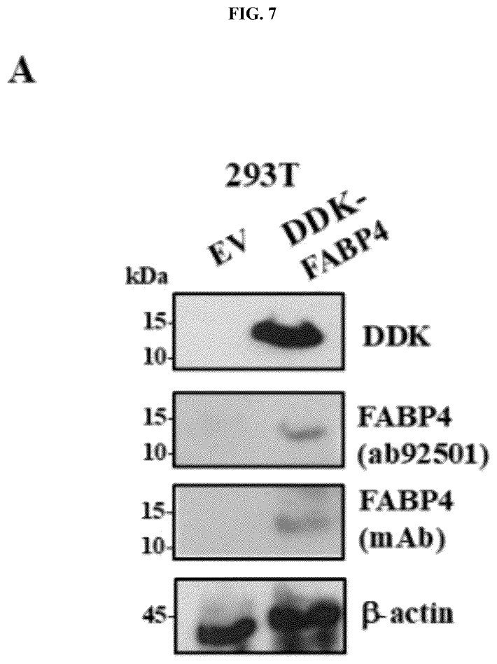

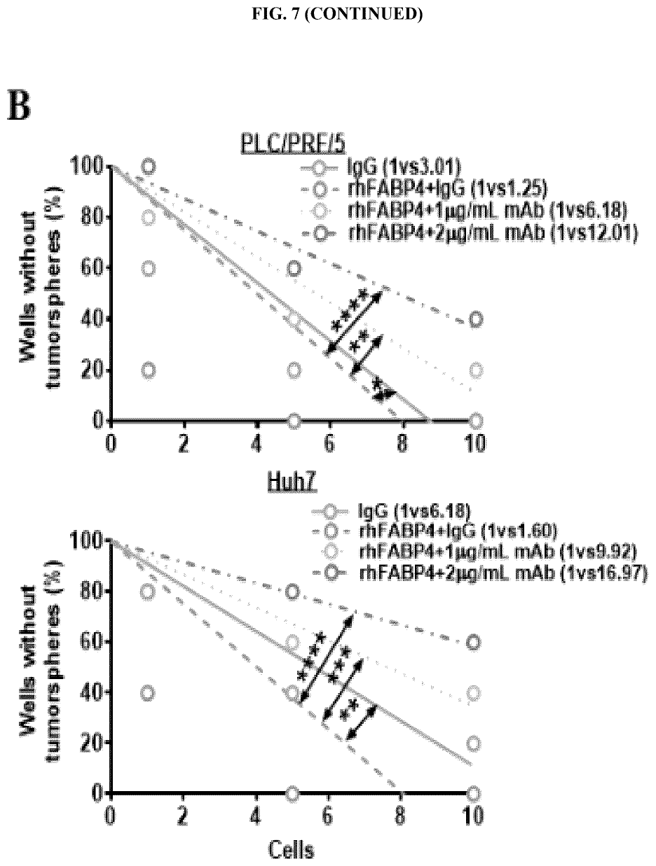

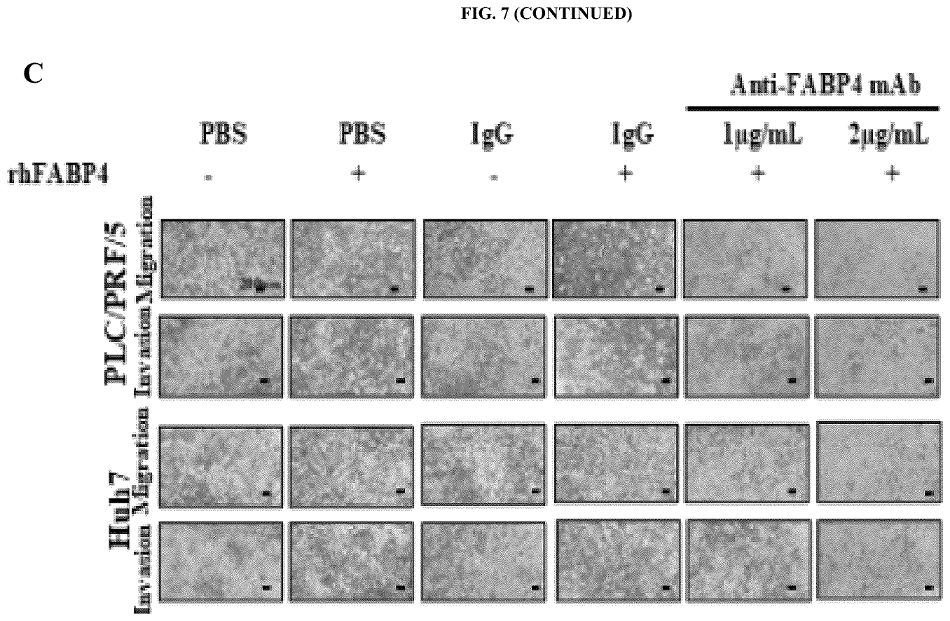

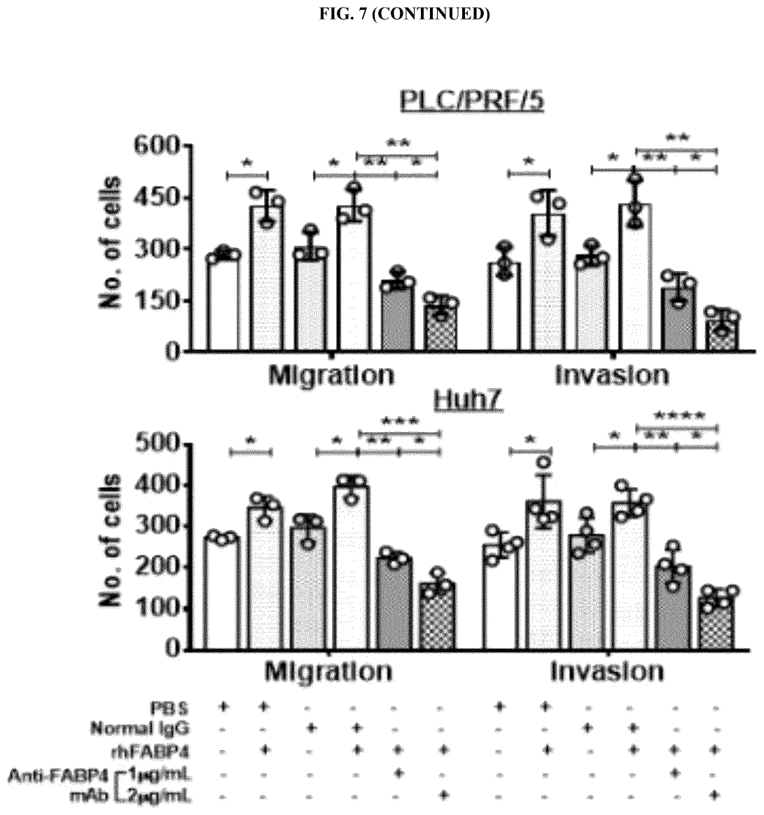

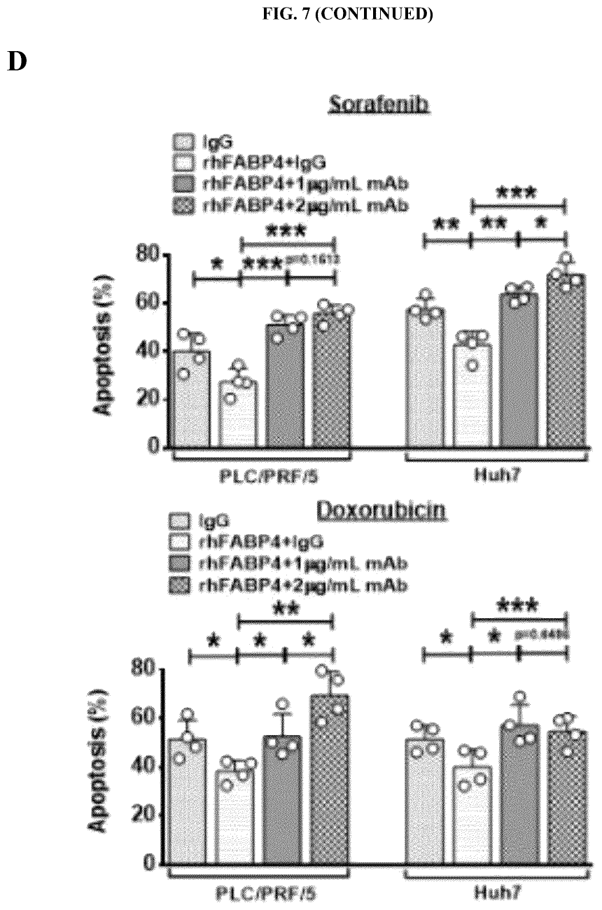

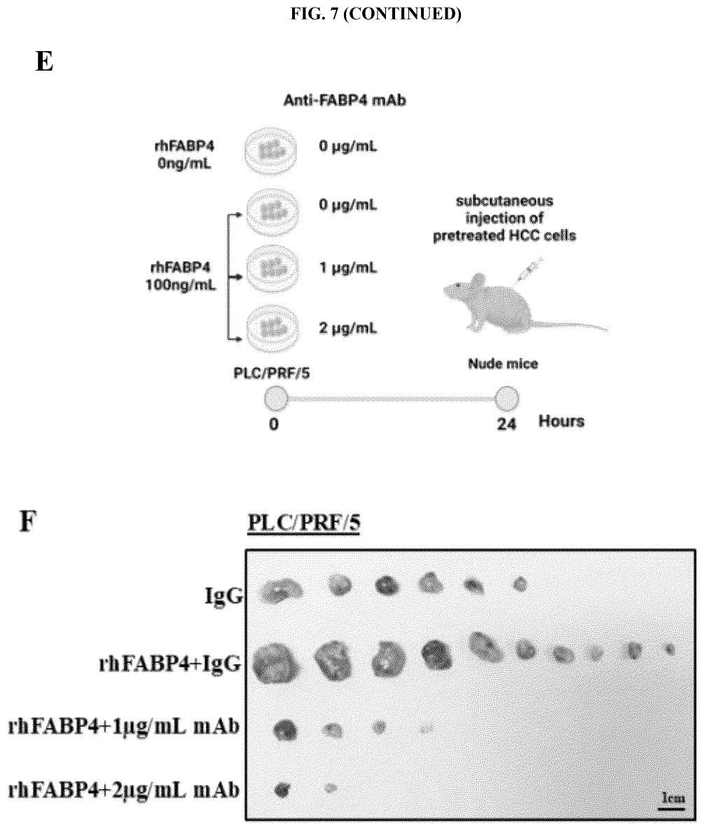

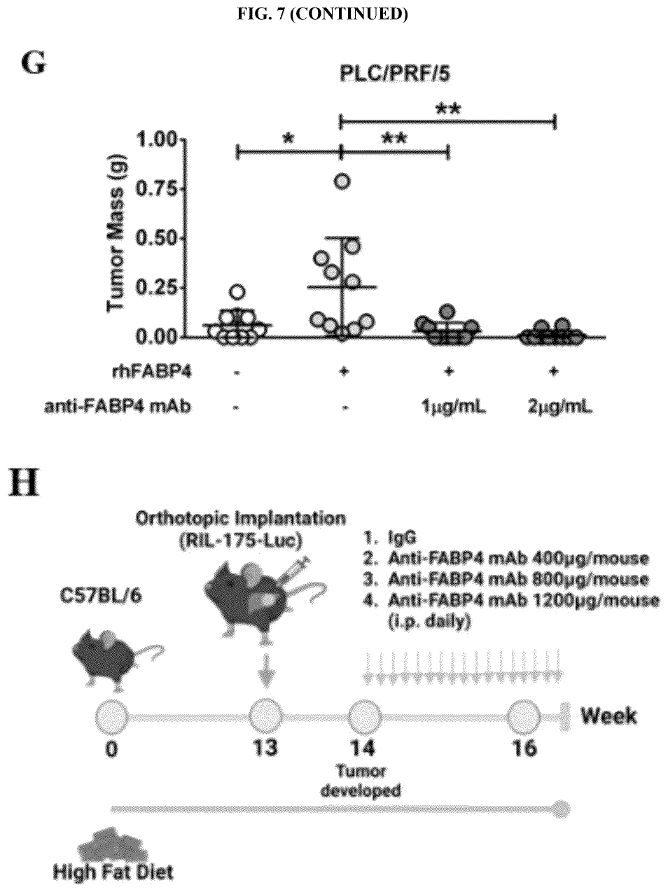

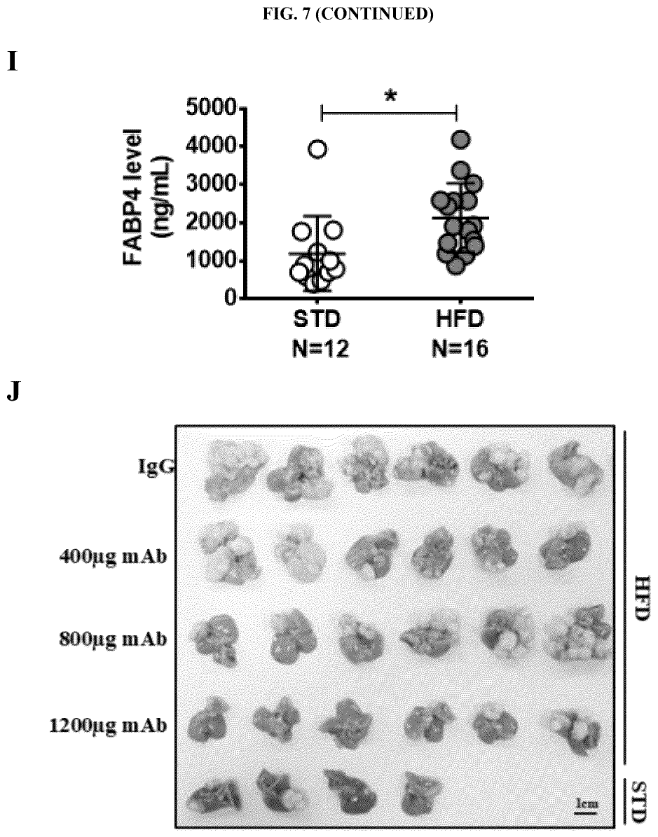

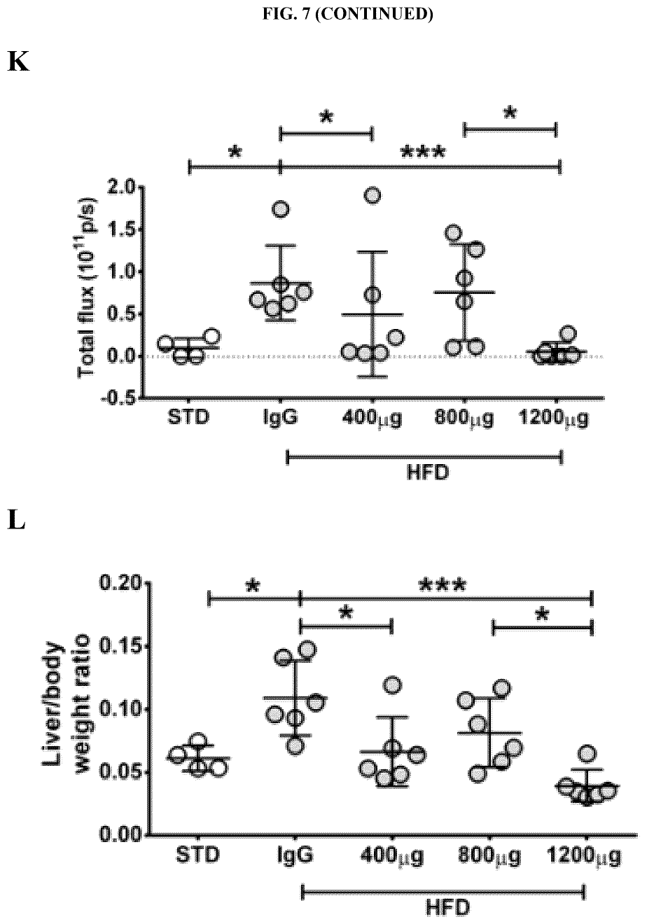

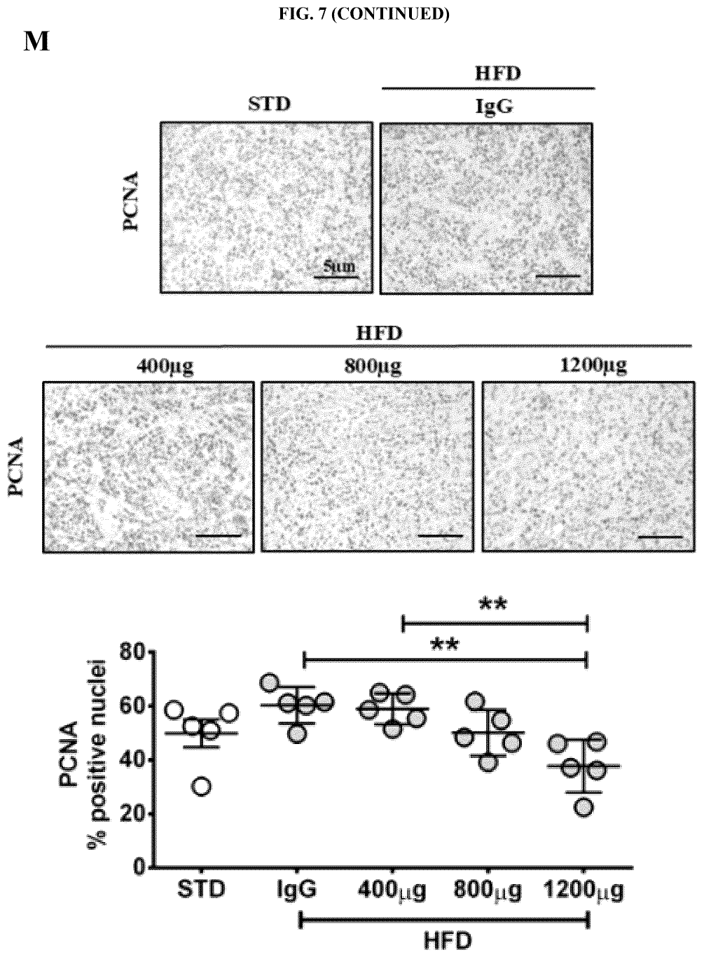

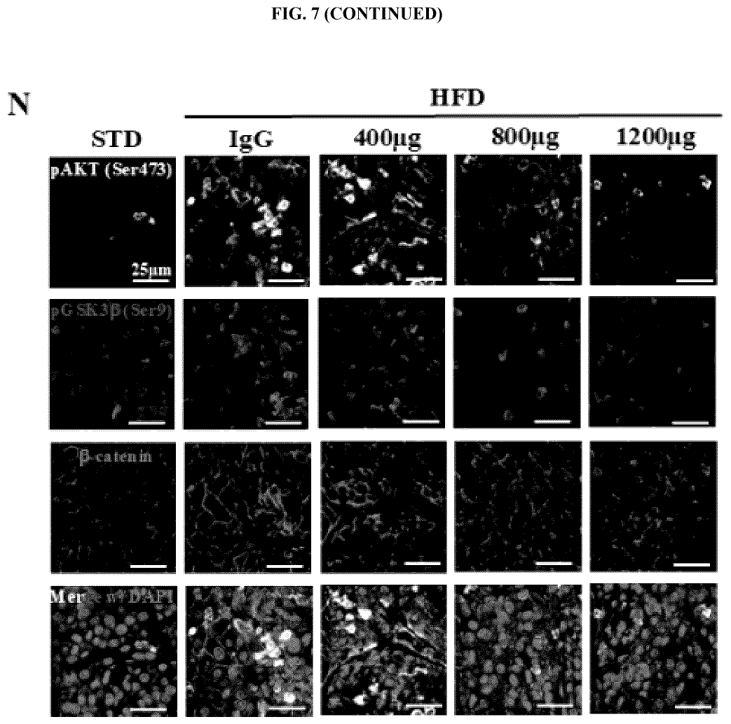

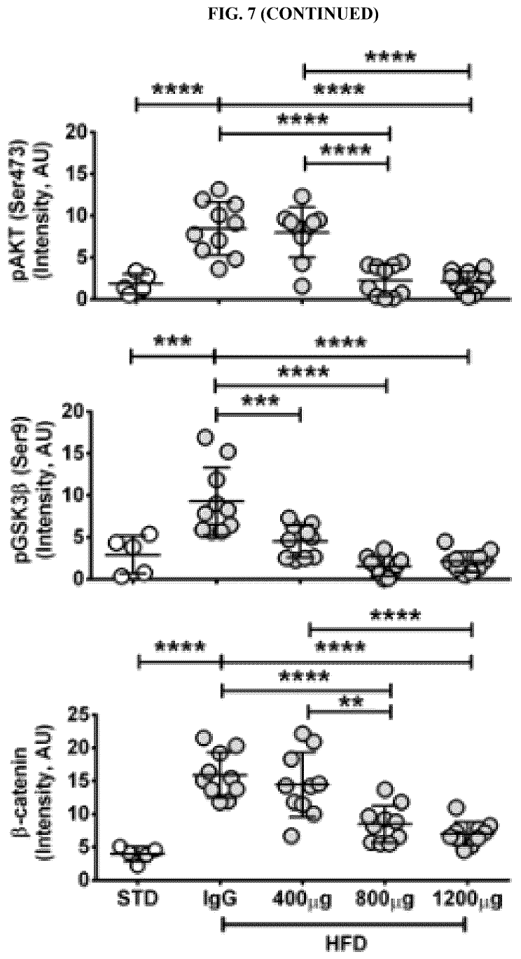

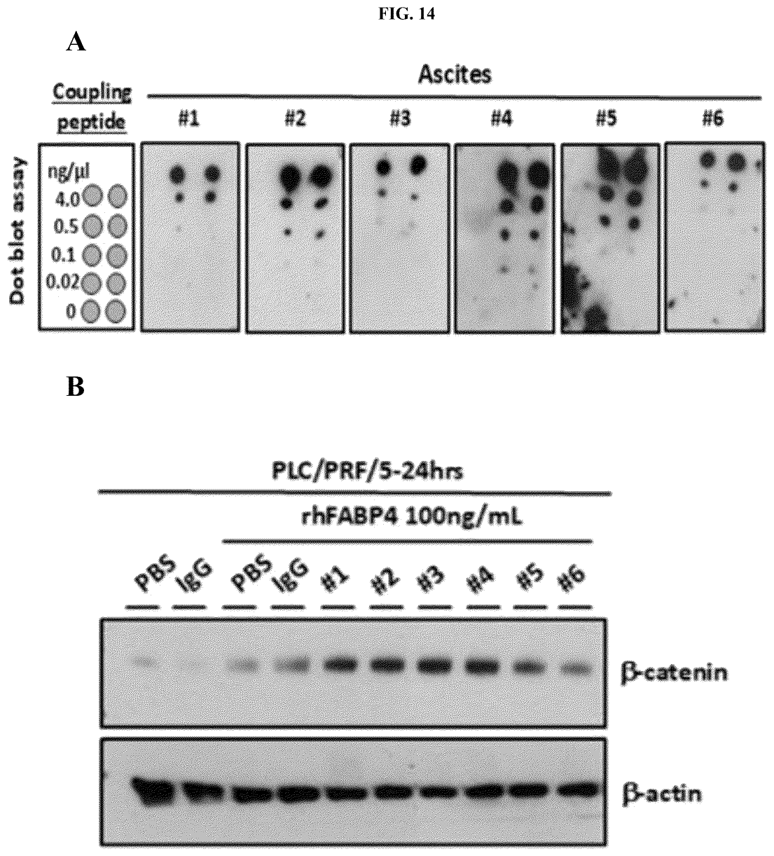

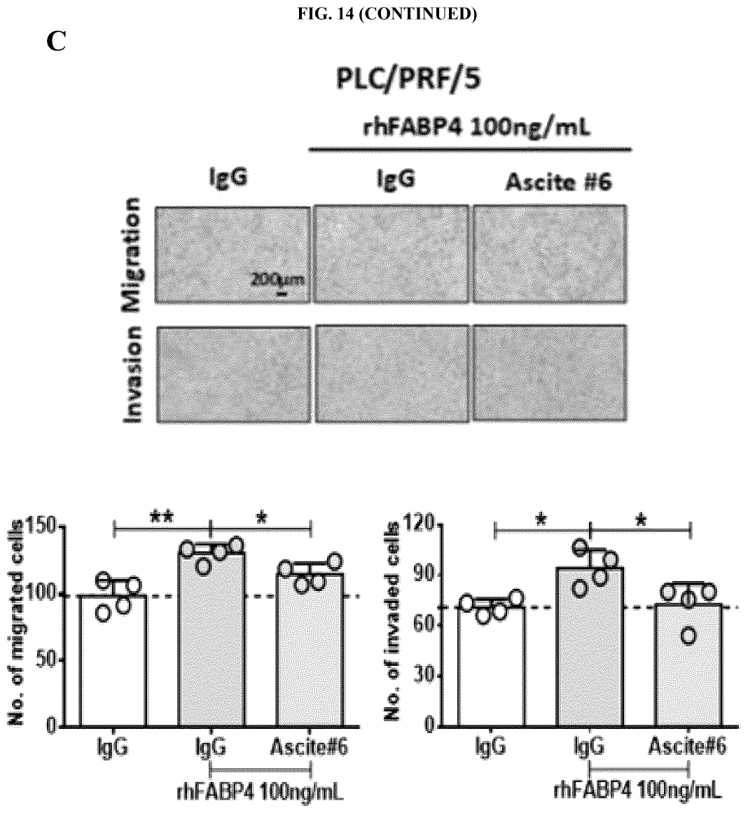

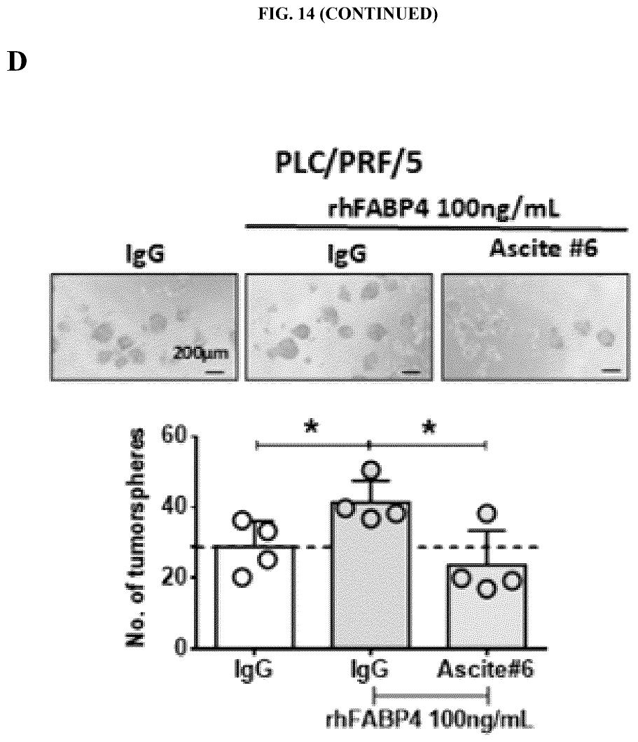

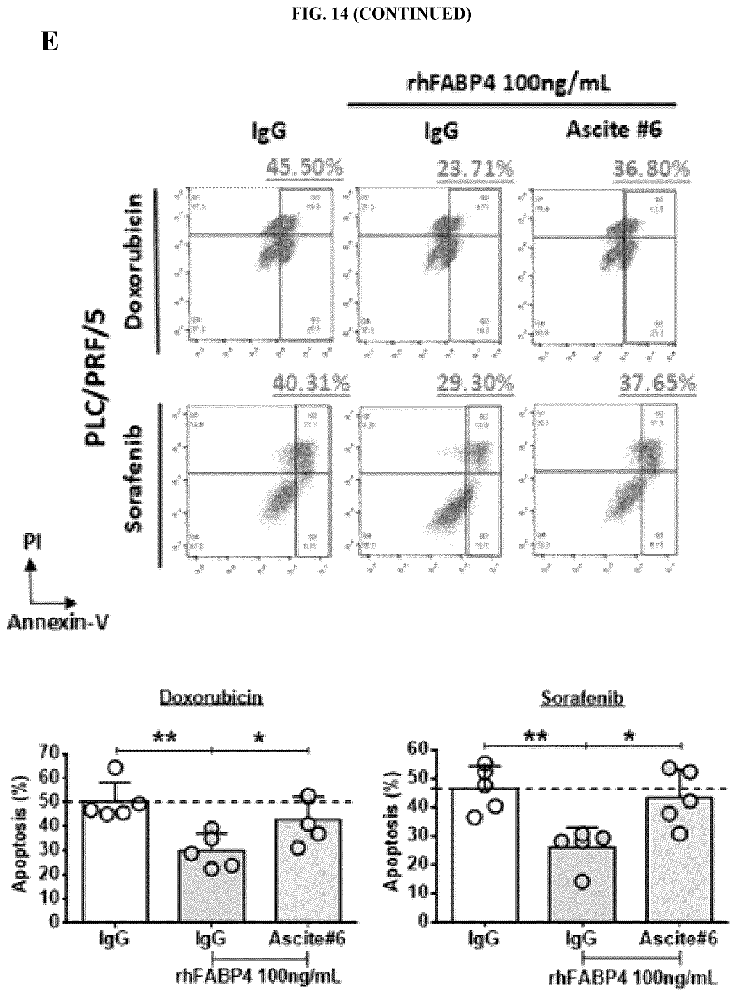

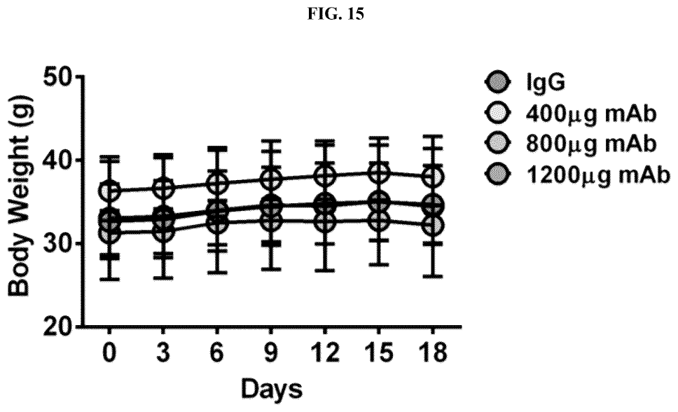

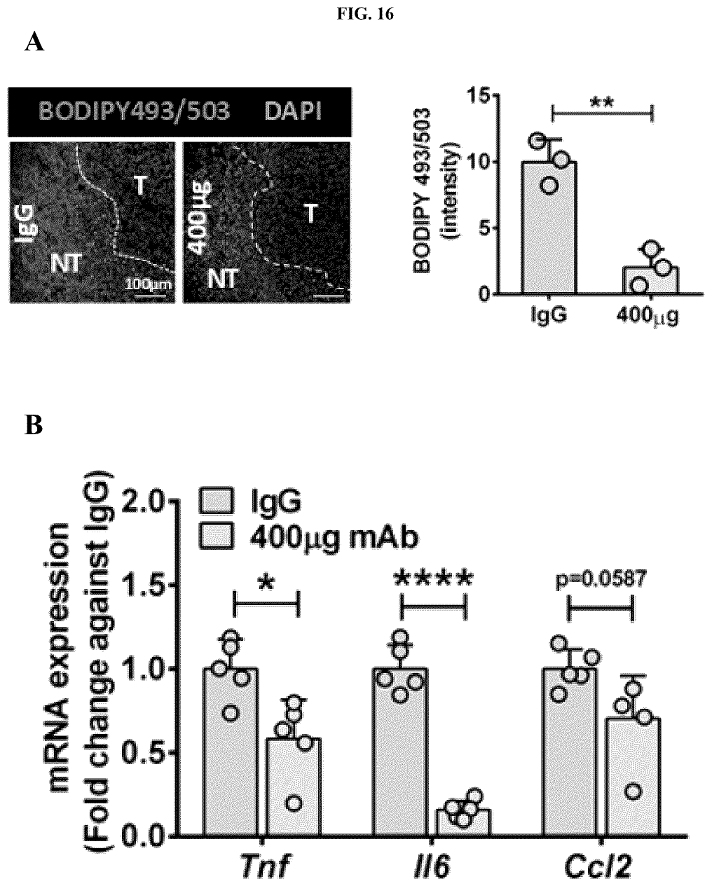

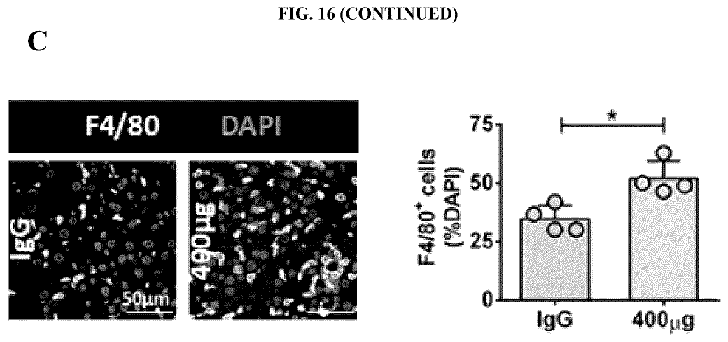

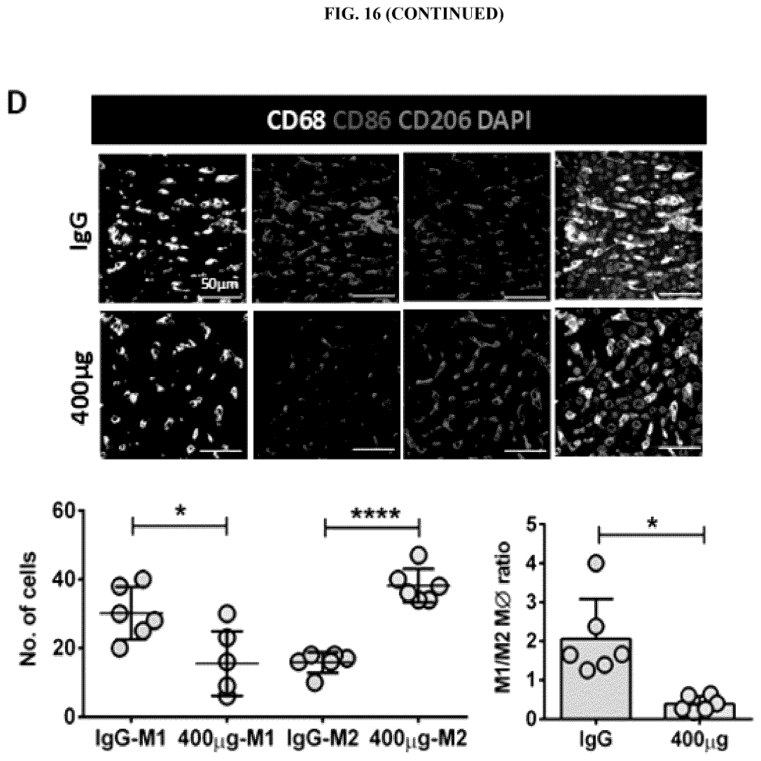

Among several ascites samples generated herein, ascites #6 showed the highest neutralising effect on rhFABP4 in hepatocellular carcinoma cells and was selected for the generation of purified anti-FABP4 monoclonal antibodies (FIG. 14). The specificity of the antibody was confirmed in DKK-tagged FABP4-overexpressing HEK 293T cells via western blotting, which revealed a clear band at approximately 12 kDa (FIG. 7A). Next, it was sought to examine the neutralising effect of this anti-FABP4 monoclonal antibody (mAb) on FABP4-driven cancer stemness. The anti-FABP4 monoclonal antibody was shown to abolish the ability of rhFABP4 to enhance self-renewal, cell migration and invasion and drug resistance (FIG. 7B to 7D). The neutralising effect of the anti-FABP4 monoclonal antibody in rhFABP4-induced tumour incidence rate and tumour growth was also shown in PLC/PRF/5 cells by co-treating these cells with 100 ng/ml rhFABP4 prior to subcutaneous injection into nude mice (FIG. 7E to 7G). Next, the therapeutic potential of the anti-FABP4 mAb in suppressing tumour growth was investigated in a MASLD-HCC mouse model induced by orthotopic injection of RIL-175 mouse hepatocellular carcinoma cells into the liver of mice fed with a high fat diet (HFD) for 13 weeks (FIG. 7H). An increase in the serum FABP4 level was seen in the high fat diet group compared with that in the standard diet (STD) group (FIG. 7I). After tumour development one week after implantation, anti-FABP4 monoclonal antibodies were administered at concentrations including, but not limited to, 400 μg, 800 μg and 1200 μg, via intraperitoneal (i.p.) injection. After treatment for 17 days, a concentration of 1200 μg of anti-FABP4 monoclonal antibody was shown to suppress tumour growth (FIG. 7J to 7L). In this time, no loss of body weight was observed in the mice (Figure S15). Upon treatment with the anti-FABP4 monoclonal antibody, hepatocellular carcinoma cells showed a decreased number of proliferating cell nuclear antigen (PCNA)-positive nuclei (FIG. 7M). Additionally, the expression levels of pAKT (Ser473), pGSK3β(Ser9), and β-catenin was shown to decrease with the introduction of anti-FABP4 antibodies at concentrations as low as 400 μg (FIG. 7N). Furthermore, FABP4 neutralization was shown to mitigate the steatotic and inflammatory states, as indicated by decreased lipid accumulation, inhibition of inflammatory gene expression, and a reduced ratio of M1/M2 macrophages (FIGS. 16A to 16D). Taken together, this data indicates that targeting the adipocyte-derived FABP4 signalling pathway is an effective treatment for MASLD-related hepatocellular carcinomas.

As used herein, the term “treatment” refers to both therapeutic treatment and prophylactic or preventative measures to address a disease or a symptom of a disease. Those in need of treatment include those already with the disease, as well as those in which the disease is to be prevented. Hence, the patient to be treated herein may have been diagnosed as having the disease, or may be predisposed, or susceptible to the disease.

As used herein, the terms “hepatocellular carcinoma” and “HCC” are used interchangeably and refer to cancer that arises from hepatocytes, the major cell type of the liver.

As used herein, the term “effective amount” refers to an amount of antibody sufficient to achieve the desired therapeutic or prophylactic effect under the conditions of administration, such as, an amount sufficient to inhibit (i.e., reduce, prevent) tumour formation, tumour growth (proliferation, size), tumour vascularization and/or tumour progression (invasion, metastasis) in the liver of a subject. The efficacy of a therapy (for example, but not limited to, reduction/elimination of a tumour and/or prevention of tumour growth) can be determined by any suitable method known in the art, such as, in situ immunohistochemistry, imaging, examples of which can be ultrasound, CT scan, MRI, NMR), and ³H-thymidine incorporation. To the extent the drug may prevent growth and/or kill existing cancer cells, it may be cytostatic and/or cytotoxic. The effective amount may extend progression free survival, result in an objective response (including a partial response (PR) or complete response (CR)), increase overall survival time, and/or improve one or more symptoms of the disease to be treated.

In one example, the effect amount is between 1 μg to 100 μg. In another example, the effective amount is at least 20 μg, at least 21 μg, at least 22 μg, at least 23 μg, at least 24 μg, at least 25 μg, at least 26 μg, at least 27 μg, at least 28 μg, at least 29 μg, at least 30 μg, at least 31 μg, at least 32 μg, at least 33 μg, at least 34 μg, at least 35 μg, at least 36 μg, at least 37 μg, at least 38 μg, at least 39 μg, at least 40 μg, at least 41 μg, at least 42 μg, at least 43 μg, at least 44 μg, at least 45 μg, at least 46 μg, at least 47 μg, at least 48 μg, at least 49 μg, at least 50 μg, at least 51 μg, at least 52 μg, at least 53 μg, at least 54 μg, at least 55 μg, at least 56 μg, at least 57 μg, at least 58 μg, at least 59 μg, or at least 60 μg. In one example, the effective amount is at least 30 μg. In another example, the effect amount is 20 μg, 20.5 μg, 21 μg, 21.5 μg, 22 μg, 22.5 μg, 23 μg, 23.5 μg, 24 μg, 24.5 μg, 25 μg, 25.5 μg, 26 μg, 26.5 μg, 27 μg, 27.5 μg, 28 μg, 28.5 μg, 29 μg, 29.5μg, 30μg, 30.5μg, 31μg, 31.5μg, 32μg, 32.5μg, 33μg, 33.5μg, 34μg, 34.5μg, 35μg, 35.5 μg, 36 μg, 36.5 μg, 37 μg, 37.5 μg, 38 μg, 38.5 μg, 39 μg, 39.5 μg, 40 μg, 40.5 μg, 41 μg, 41.5 μg, 42 μg, 42.5 μg, 43 μg, 43.5 μg, 44 μg, 44.5 μg, 45 μg, 45.5 μg, 46 μg, 46.5 μg, 47 μg, 47.5 μg, 48 μg, 48.5 μg, 49 μg, 49.5 μg, 50 μg, 50.5 μg, 51 μg, 51.5 μg, 52 μg, 52.5 μg, 53 μg, 53.5 μg, 54 μg, 54.5 μg, 55 μg, 55.5 μg, 56 μg, 56.5 μg, 57 μg, 57.5 μg, 58 μg, 58.5 μg, 59 μg, 59.5 μg, or 60 μg. In one example, the effective amount is 32.5 μg.

In one example, the antibody disclosed herein can be administered once, twice, thrice, or multiple times to a subject. In another example, the antibody disclosed herein is administered to the subject at daily, weekly, bi-weekly (i.e., twice a week), monthly, or bi-monthly (i.e., twice a month) intervals.

It was shown herein that adipocytes regulate liver cancer stem cell (CSC) functions via paracrine secretion and that this effect is further enhanced when adipocytes are co-cultured with hepatocellular carcinoma cells. This result is consistent with a previous report showing the role of adipocytes in promoting tumour growth, metastasis and drug resistance in ovarian cancer. In MASLD, steatosis triggers inflammation by recruiting and activating macrophages. The chronic inflammatory state leads to the progression of this disease to hepatocellular carcinoma. Notably, when differentiated macrophages derived from THP1 were exposed to ADCM-hepatocellular carcinoma conditioned media, these stimulated macrophages were observed to contribute to increased sphere formation, indicative of increase in cancer stemness (data not shown). Nonetheless, the enhancing effect conferred by stimulated adipocytes was more pronounced. This finding further reinforces the role of adipocytes in conferring cancer stem cell properties to hepatocellular carcinoma cells. Mass spectrometry revealed that FABP4 was enriched in adipocytes co-cultured with hepatocellular carcinoma cells, while a statistically significant amount of FABP4 was detected in the serum of MASLD-HCC patients. This is in line with previous reports showing increased circulating FABP4 in individuals with obesity and in individuals with decompensated cirrhosis. In addition, MASLD patients have been shown to have an increase in serum FABP4 levels compared with those without liver disease, and this upregulation has been shown to persist in MASLD-HCC patients. Elevated levels of serum FABP4 have also been reported in obesity-associated breast cancer patients compared to non-obese patients.

Circulating FABP4 promoted obesity-associated breast cancer by increasing mammary tumour stemness and aggressiveness through the IL-6/STAT3/ALDH1 axis. Subsequently, it has been reported that FABP4 depletion suppresses the activation of stemness properties in colorectal cancer via the modulation of the ERK/mTOR pathway. However, the functional and mechanistic roles of adipose-derived FABP4 in cancer stemness remain unknown. By exogenously administering rhFABP4, it was demonstrated that FABP4 is involved in the regulation of liver cancer stem cells. This data, together with the suppression of the cancer stem cell-enhancing effect of adipocytes by BMS-309403, indicate that adipose-derived FABP4 plays a role in the regulation of liver cancer stem cells. In addition, FABP4 also confers resistance to doxorubicin and sorafenib, which is consistent with findings showing the role of FABP4 in driving drug resistance in ovarian cancer. Using Fabp4^{-/-} mice, the oncogenic role of FABP4 in MASLD-induced hepatocellular carcinoma was shown. Without being bound by theory, it is thought that the delayed oncogenic effect could be due to the loss of FABP4 in liver as well as the distinct visceral adipocytes. Bulk RNA sequencing analysis showed that exogenous FABP4 regulates liver cancer stemness through the PI3K/AKT/β-catenin signalling pathway. It had previously been shown that CD36 as a direct binding partner of FABP4, facilitating fatty acid transfer from adipocytes to breast cancer cells. The role of CD36 suppression in modulating β-catenin signalling was analysed but found to be inconclusive. Therefore, streptavidin capture-based mass spectrometry with biotin-labelled rhFABP4 to identify possible membrane binding targets of FABP4 in hepatocellular carcinoma cells.

ITGB1 was identified as a membrane receptor that directly interacts with rhFABP4 and is shown to be overexpressed in cancers, such as gastric and breast cancers, correlating with poorer clinical outcomes. Analysis of the TCGA-LIHC cohort showed that hepatocellular carcinoma patients with MASLD, a condition linked to high ITGB1 expression, had worse overall survival compared to those with low ITGB1 expression. ITGB1 is shown to promote cell proliferation and migration in colorectal cancer and to sensitizes hepatocellular carcinoma cells to sorafenib treatment when ablated. Findings disclosed herein indicated the role of ITGB1 in linking FABP4 with the AKT/β-catenin signalling pathway, as shown by inhibited cancer stem cell-promoting and AKT/β-catenin signalling in ITGB1-suppressed cells in the presence of rhFABP4. Analysis of MASLD-HCC and MASLD patient datasets with high hepatocellular carcinoma risk showed a statistically significant, positive correlation between FABP4 and either ITGB1 or CTNNB1, supporting the role of ITGB1 in adipocyte-derived FABP4-driven MASLD-hepatocellular carcinoma.

Strategies targeting FABP4 had been previously reported to treat acute liver injury and non-alcoholic steatohepatitis in mice via the pharmacological inhibitor BMS-309403. However, this inhibitor comes with severe side effects, such as an induced acute cardiac depressant effect, which limits its clinical use. An anti-FABP4 monoclonal antibody has been developed herein which not only demonstrated the required specificity, but also was shown to attenuate the cancer stem cell-enhancing effects of rhFABP4, together with the suppression of the AKT/β-catenin pathway. Pretreatment with the FABP4 monoclonal antibody in hepatocellular carcinoma (HCC) cells also impeded the rhFABP4-driven tumorigenic effect. As shown herein, administration of 1200 μg of anti-FABP4 monoclonal antibody for 17 days was shown to suppress tumour growth in an orthotopic MASLD-HCC mouse model, which was accompanied by suppression of the AKT/β-catenin signalling pathway. Furthermore, blockage of FABP4 action, either through genetic ablation or neutralisation, was shown to lead to suppression of steatosis and inflammation, which can attenuate hepatocellular carcinoma tumour growth.

The antibodies disclosed herein can be administered, for example, by intravenous, intraarterial, or intraperitoneal means for prophylactic and/or therapeutic treatment. The most typical route of administration of an antibody is intravascular, subcutaneous, or intramuscular, although other routes can be effective. In some methods, antibodies are administered as a sustained release composition or device, where appropriate.

Effective doses of the antibody of the present invention, for the treatment of the conditions described herein can vary depending upon many different factors, including means of administration, target site, physiological state of the subject, whether the subject is human or an animal, other medications administered, and whether treatment is prophylactic or therapeutic. In one example, the subject is a human. In another example, the subject is a non-human mammal.

The dosage and frequency of administration can vary depending on whether the treatment is prophylactic or therapeutic. In therapeutic applications, a relatively high dosage at relatively short intervals is sometimes required until progression of the disease is reduced or terminated, and preferably until the subject shows partial or complete amelioration of symptoms of disease.

Treatment of a subject suffering from a disease or disorder can be monitored using standard methods. Some methods entail determining a baseline value, for example, of a protein level or profile in a subject, before administering a dosage of agent, and comparing this with a value for the profile or level after treatment. A significant increase (i.e., greater than the typical margin of experimental error in repeat measurements of the same sample, expressed as one standard deviation from the mean of such measurements) in value of the level or profile signals a positive treatment outcome (i.e., that administration of the agent has achieved a desired response). If the value for immune response does not change significantly, or decreases, a negative treatment outcome is indicated.

In other methods, a control value (i.e., a mean and standard deviation) of level or profile is determined for a control population. Typically, the individuals in the control population have not received prior treatment. Measured values of the level or profile in a subject after administering a therapeutic agent arc then compared with the control value. A significant increase relative to the control value (e.g., greater than one standard deviation from the mean) signals a positive or sufficient treatment outcome. A lack of significant increase or a decrease signals a negative or insufficient treatment outcome.

The therapeutic compositions of the invention include at least one of the antibodies produced by a method described herein in a pharmaceutically acceptable carrier. A “pharmaceutically acceptable carrier” refers to at least one component of a pharmaceutical preparation that is normally used for administration of active ingredients. As such, a carrier may contain any pharmaceutical excipient used in the art and any form of vehicle for administration. The compositions may be, for example, injectable solutions, aqueous suspensions or solutions, non-aqueous suspensions or solutions, solid and liquid oral formulations, salves, gels, ointments, intradermal patches, creams, lotions, tablets, capsules, sustained release formulations, and the like. Additional excipients may include, for example, colorants, taste-masking agents, solubility aids, suspension agents, compressing agents, enteric coatings, sustained release aids, and the like. The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or non-toxic, non-therapeutic, non-immunogenic stabilizers and the like.

Typically, compositions are prepared as injectables, either as liquid solutions or suspensions; solid forms, such as a lyophilised powder, suitable for solution in, or suspension in, liquid vehicles prior to injection can also be prepared.

As used herein, the term “subject” refers to a mammalian subject. The term “mammalian subject” can include mammals such as, but not limited to, primates (for example, humans and monkeys), cows, sheep, goats, horses, dogs, cats, rabbits, guinea pigs, rats, mice; or other bovine, ovine, equine, canine feline, rodent or murine species. Examples of subjects include, but are not limited to, human patients who have, or are at risk for developing, hepatocellular carcinoma.

The role of adipocyte-derived FABP4 in the regulation of cancer stemness in hepatocellular carcinoma (HCC) is shown, and its effect were enhanced by co-culture with hepatocellular carcinoma cells. The FABP4-ITGB1-AKT/β-catenin pathway is a signalling cascade driving MASLD-related hepatocellular carcinoma. Targeting secretory FABP4 with an anti-FABP4 monoclonal antibody was shown to impede the growth of MASLD-hepatocellular carcinoma tumours, making it a viable option for treating hepatocellular carcinoma (HCC) and MASLD-related hepatocellular carcinoma.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

In summary, the methods shown herein were used to evaluate the interaction between differentiated adipocytes and HCC cells using a co-culture system. The conditioned medium of the adipocytes was profiled using Orbitrap liquid chromatography mass spectrometry. The functional role of FABP4 in the regulation of liver cancer stem cells was evaluated by various cancer stem cells functional assays. In vivo functional characterization of FABP4 was performed in Fabp4 knockout mice. Molecular pathways mediating phenotypic alterations were identified using mass spectrometry, RNA sequencing analysis, and pathway analysis. The therapeutic efficacy of monoclonal neutralising antibody against FABP4 was evaluated in two mouse models, a hepatocellular carcinoma (HCC) xenograft model and a MASLD-HCC model.

Sex as a biological variable—The experiments performed herein examined human samples of both male and female subjects. Experiments were conducted on female and male mice in separate studies. Sex was not considered as a biological variable. While the data disclosed herein focused on male mice, the findings can be relevant to both sexes.

Cell lines and cell culture—The following cells lines were maintained in Dulbecco's Modified Eagle Medium (DMEM) with high glucose and L-glutamine (Gibco, Invitrogen) supplemented with 10% heat-inactivated fetal bovine serum (FBS) (Gibco, Invitrogen), 100 mg/mL penicillin G and 50 μg/mL streptomycin (Gibco, Invitrogen) at 37° C. in a humidified chamber containing 5% CO2: HSC-hTERT (America Type Culture Collection, Manassas, VA, USA); Huh7 and PLC/PRF/5 human hepatocellular carcinoma (HCC) cell lines (Japan Cancer Research Bank) and 293 FT (Invitrogen, Thermo Fisher Scientific, Waltham, USA). The culture medium was refreshed every 2 days. Human visceral preadipocyte (PT5005, Lonza) was maintained in preadipocyte basal medium supplemented with fetal bovine serum, L-glutamine, gentamicin sulphate and amphotericin-B from Lonza. Preadipocytes were differentiated into adipocytes using preadipocyte basal medium supplemented with recombinant human insulin, dexamethasone, indomethacin and IBMX according to manufacturer protocol. Morphological changes in adipocytes were observed within a week of differentiation and incubation with differentiation medium for 20 days resulted in 70% differentiation of preadipocytes to adipocytes with lipid accumulation. All cell lines used in herein obtained between 2013 and 2016, and were authenticated by morphological observation and STR DNA analysis with PowerPlex 16HS kit (Promega) as well as tested for the absence of mycoplasma contamination (MycoAlert, Lonza). Cells were used within 20 passages after thawing.

Patient Samples—Venous blood samples were collected from 27 individuals diagnosed with MASLD-related hepatocellular carcinoma before any therapeutic interventions had been performed. Tumours and paired non-tumorous liver tissue samples from 17 patients diagnosed with hepatocellular carcinoma and MASH were obtained from Prince of Wales Hospital, The Chinese University of Hong Kong, and Zhongshan Hospital of Fudan University.

Flow cytometric analysis—Cells were stained by Phycoerythrin (PE)-conjugated CD47 and CD90 antibodies (#536046 & #555596, BD Biosciences) in PBS with 2% FBS at 4° C. for 30 to 60 minutes. Isotype-matched mouse immunoglobulins served as controls. Samples were analysed using BD Accuri C6 flow cytometer and FACSDiva software (BD Biosciences).

Anti-FABP4 monoclonal antibody production—Anti-FABP4 monoclonal antibody (also referred to herein as “3I19-1”, which targets the ligand binding region of FABP4 (AbMart, Shanghai, China), was synthesized against the epitope peptide GQEFDEVTADDRKV (SEQ ID NO: 1; amino acids 68-81 of human FABP4).

Development of MASLD-hepatocellular carcinoma model—Fabp4 knockout (KO) mice in C57BL/6N background were generated using procedures as previously described. Age-matched, male Fabp4 KO mice and their littermates were used. Animals were allocated to their experimental group according to their genotypes. The investigators were not blinded to the experimental groups. For chemically induced MASLD-HCC model, male wildtype C57BL/6N and Fabp4 KO mice were treated with N-nitrosodiethylamine at 25 mg/kg intraperitoneally (DEN, Sigma-Aldrich) at the age of 14 days. Starting at 6 weeks of age, the mice were fed with high fat diet (D12492, Research Diets, USA) for 29 weeks. Serum and liver samples of mice were collected for analysis at the end point. Tumour nodules in the livers were counted by visual inspection.

The effect of FABP4 antibody in MASLD-hepatocellular carcinoma model—An orthotopic MASLD-hepatocellular carcinoma mouse model was established using female C57BL/6J. High fat diet (HFD) was provided to the mice at 4-week-old for 13 weeks prior to tumour cell inoculation. Seven thousand five hundred luciferase-labelled mouse hepatocellular carcinoma RIL-175 cells, re-suspended in 50% matrigel, were orthotopically injected at the left lobes of the livers. The tumour growth was monitored by the IVIS imaging system (Perkin-Elmer). Either isotypic IgG control or anti-FABP4 monoclonal antibody, at an amount of 400 μg, 800 μg or 1200 μg per mouse, was injected intraperitoneally, once daily, for 17 days. Standard diet (STD) (#5053, Rodent Diet 20, PicoLab) group was setup as control. In addition, PLC/PRF/5 cells were pretreated with rhFABP4 at 100 ng/ml, with either 1 μg/mL or 2 μg/mL of anti-FABP4 monoclonal antibody, for 24 hours prior to subcutaneously inoculation in nude mice. Mice were sacrificed if the percentage of body weight loss is greater than 20% including the tumour mass.

Statistical analysis—The statistical significance of the results obtained from limiting dilution assay, flow cytometric analysis, migration and invasion assay, qRT-PCR, immunohistochemical and immunofluorescence staining, in vivo tumour growth and volume were determined by two-tailed t-test, where appropriate using GraphPad Prism (GraphPad Software, San Diego, CA). One-way ANOVA analysis was applied, where appropriate. The results were presented as means and standard deviations, and p-values of less than 0.05 were considered statistically significant. A data point was excluded if it deviated from the mean by more than three standard deviations. Investigators were not blinded to the group allocation during the experiment or when assessing the outcome in all experiments, including animal experiments. There was no estimate of variation within each group of data. The variance was similar between the groups that were being statistically compared. Kaplan-Meier survival analysis was used to analyse overall survival, and a log-rank test was used to determine the statistical significance.

Study approval—Human MASLD-hepatocellular carcinoma tumour tissue and adjacent normal tissue were collected in Prince of Wales Hospital, the Chinese University of Hong Kong, from biopsy-proven MASLD-hepatocellular carcinoma (n=17). Venous blood samples were collected from 27 MASLD-related hepatocellular carcinoma patients, which was collected from hepatocellular carcinoma patients before any therapeutic procedures had been performed.

Data Availability—Bulk RNA sequencing data for PLC/PRF/5 cells, untreated (rhFABP4_0 ng) and untreated with rhFABP4 at 100 ng/ml (rhFABP4_100 ng), as well as hepatocellular carcinoma tumours derived from wild-type (Wildtype) and Fabp4 knockout (Fabp4Knockout) mice, have been deposited in the Gene Expression Omnibus under accession numbers GSE292914 and GSE286130, respectively.

Assessment of steatosis and lobular inflammation scores—The steatosis and lobular inflammation scores of the mouse livers were assessed using Haematoxylin & Eosin (H&E) staining. Steatosis was scored according to the following criteria validated by the Pathology Committee of the NASH Clinical Research Network 1:0 (<5%), 1 (5-33%), 2 (>33-66%) or 3 (>66%) of parenchymal involvement of steatosis. Inflammation was scored by overall assessment of all inflammatory foci according to the following criteria: 0 (No foci), 1 (<2 foci per field), 2 (2-4 foci per field) or 3 (>4 foci per field). Five random fields were assessed in each sample.

Hepatocellular carcinoma patient-derived organoids and culture conditions—Hepatocellular carcinoma tissue was obtained from patients undergoing hepatectomy at Queen Mary Hospital, Hong Kong. Samples were collected from patients who had not received any previous local or systemic treatment prior to operation. For organoid cultures, cells were isolated and cultured according to protocol. Hepatocellular carcinoma patient-derived organoids labelled HCC #23 was gift from Dr. Meritxell Huch of The Gudon Institute at the University of Cambridge.

Oil Red-O staining—Preadipocytes and adipocytes seeded on coverslips were fixed in 4% paraformaldehyde (PFA) for 10 minutes prior to twice of PBS washings. The fixed cells were incubated with isopropanol for 5 minutes and stained with Oil Red-O solution (Sigma Aldrich) for 15 minutes. Cells were counterstained with haematoxylin for 5 minutes. The stained cells were washed twice with PBS and subjected to microscopic examination.

BODIPYTM 493/503 staining for neutral lipid droplets—Frozen mouse liver tissue was fixed with 4% PFA for 10 minutes following two rounds of washing with PBS. The neutral lipid droplets were stained using BODIPYTM 493/503 (Invitrogen, D3922) at 1 μg/mL for 1 hour at 37° C. Cells were washed with PBS. Slides were counterstained with DAPI for nuclei, mounted, and subjected to Leica TCS SPE confocal microscope examination. The fluorescent intensity of lipid droplets was qualified using ImageJ.

Collection of conditioned media (CM)—Preadipocytes, adipocytes, HSC-hTERT and hepatocellular carcinoma cells were seeded in 6-well plates and grown to 80% confluency. Culture medium was removed, and 1.5 ml serum free media was added per well for incubation of 72 hours at 37° C. The conditioned media was centrifuged at 3,000 rpm at 4° C. for 5 minutes and the supernatant collected.

Knockdown of ITGB1 and CTNNB1—To establish ITGB1 and CTNNB1 knockdown clones, lentiviral particles were generated by co-transfecting 293 FT cells with shITGB1 or shCTNNB1 cloned using pLKO.1-puro lentiviral vector or non-target control (NTC) plasmids and packaging plasmid mix. Viral supernatant was collected for infection of PLC/PRF/5 and Huh7. The shRNA sequences were listed in the sequence listing table.

Cell viability assay—Cell viability of organoid cultures treated with specified concentrations of rhFABP4 for 6 days was evaluated using CellTiter-Glo® Luminescent Cell Viability Assay (Promega) according to manufacturer's protocol.

Sphere formation assay—Three to five hundred cells of PLC/PRF/5, Huh7 and hepatocellular carcinoma organoids (HK-HCC P1 and HCC #23) were seeded to 24-well plates coated with poly (2-hydroxyethyl methacrylate) (polyHEMA, Sigma Aldrich). Cells were grown in 500 μl of either conditioned media or serum-free 0.25% methylcellulose (Sigma-Aldrich) DMEM/F12 medium supplemented with 4 μg/mL insulin (Sigma-Aldrich), B27 (Invitrogen) and recombinant human FABP4 (rhFABP4, Biovision) at a concentration of either 20 ng/ml or 100 ng/ml. The number of tumourspheres (diameter larger than 100 μm) was counted under microscope after 8 to 12 days.

For sphere formation assay using co-culturing system, 4×10⁴ adipocytes or preadipocytes were seeded into an insert with pore size of 0.4 μm (Merck Millipore, Germany) while 300 to 500 hepatocellular carcinoma cells were seeded in the lower chamber in 500 μl serum-free 0.25% methylcellulose (Sigma-Aldrich) DMEM/F12 medium. The number of tumourspheres was counted after 10 days.

Limiting dilution assay—Parental or shITGB1 PLC/PRF/5 and Huh7 cells were cultured in a 96-well plate coated with polyHEMA with 200 μl serum-free 0.25% methylcellulose DMEM/F12 medium supplemented with 4 μg/mL insulin, B27 and rhFABP4 when appropriate. Sphere formation was scored after 8 to 12 days under a phase contrast microscope. The frequency of sphere-forming cell was calculated accordingly using an extreme limiting dilution algorithm (ELDA).

Migration and invasion assays—Migration assays were performed using polycarbonate membrane transwell inserts with pore size of 8 μm (Millipore). Invasion assays were performed using self-coated Matrigel (BD Biosciences) transwell inserts. Hepatocellular carcinoma cell lines (PLC/PRF/5 and Huh7) or patient-derived organoids (HK-HCC P1 and HCC #23) in serum-free medium were seeded in upper chamber, while the lower chamber was supplemented with either ADCM or 100 ng/mL rhFABP4 in serum-free medium as a chemoattractant. Serum-free DMEM was used as a negative control. Cells were incubated in humidified incubation at 37° C. for 48 hours. The transwell membranes were fixed with methanol and stained with 1% crystal violet. The membranes were cleaned and air-dried. Photographs of five randomly selected fields of the fixed cells were captured and the cells were counted using ImageJ.

Enzyme linked immunosorbent assay (ELISA)—Serum FABP4 from patients and secretory FABP4 in conditioned media in human cell lines was quantified using the human FABP4 Duo Set ELISA kit (DY3150-05, R&D Systems). FABP4 and AFP levels in mouse sera were quantified using mouse FABP4 ELISA kit (RD291036200R, BioVendor) and mouse AFP Quantikine ELISA kit (MAFP00, R&D Systems). Quantitation was performed according to manufacturers' protocols.

Biotinylation of rhFABP4 and Streptavidin capturing of membrane protein—rhFABP4 was biotin-labelled and crosslinked with sulfosuccinimidyl-2-[6-(biotinamido)-2-(p-azidobenzamido) hyxanoamido]ethyl-1,3′-ithiopropionate) (Sulfo-SBED, A39260, Thermo Fisher Scientific) according to manufacturer's instructions. In brief, 50 μg of rhFABP4 (#4504, BioVision) was incubated with 1 mg of dissolved Sulfo-SBED at room temperature for 30 minutes. The reaction mixture was desalted using Amicon® Ultra-0.5 centrifugal filter device with a 3,000 Molecular Weight Cut Off (Millipore, Germany). The desalted biotinylated rhFABP4 was incubated in PBS with adherent Huh7 cells at room temperature for 10 minutes. The cell surface proteins interacting with biotinylated rhFAPB4 was captured by the photoreactive aryl azide moiety using long wave-UV at 360 nm. The labelled proteins were isolated by NeutrAvidin Agarose slurry and reduced using Pierce™ Cell Surface Protein Biotinylation and Isolation Kit (#A44390, Thermo Fisher Scientific). The interacting rhFABP4-surface protein complex was isolated, and the disulfide bond reduced, resulting in the biotin label being transferred to the interacting proteins.

Liquid Chromatography with tandem mass spectrometry (LC-MS/MS) analysis—The purified samples were prepared for mass spectrometry using EasyPep™ Mini MS Sample Prep Kit (#A40006, Thermo Scientific) according to manufacturer's protocol. Eluted peptides were subjected to LC-MS/MS analyses performed on an Orbitrap Fusion Lumos Mass Spectrometer (Thermo Fisher Scientific) coupled with a Dionex UltiMate™ 3000 RSLnano system (Thermo Fisher Scientific). Peptides were first trapped with a 1 mm i.d.×5 mm length trap cartridge (Thermo Fisher Scientific) for 10 minutes with trapping flow of 10 μl per minute, and were then separated on a PepMap C18 column (75 μm i.d.×25 cm length) with 2 μm particle size (Thermo Fisher Scientific). Mobile phases A and B consisted of 0.1% formic acid (FA) in water and 0.1% formic acid in acetonitrile (CAN), respectively. The liquid chromatography (LC) gradient for separation with a flow rate of 300 nl per minute is as follow: mobile phase B at 6% for 12 minutes; mobile phase B was increased to 20% at 82 minutes and 30% at 92 minutes, followed by 90% mobile phase B at 100 minutes and held for 5 minutes; and mobile phase B was returned to 2% at 105 minutes and maintained until 120 minutes. Both trapping and separation are performed in a column oven at 50° C. Data was collected in data dependent acquisition (DDA) mode. The precursor ions with a charge state of 2+ or higher were fragmented by higher-energy collisional dissociation (HCD) with normalized collision energy of 30%. The MS1 Orbitrap resolution was set at 60,000 with standard AGC target in the scan range of 400 to 1500 m/z and maximum injection time of 20 milliseconds. Dynamic exclusion time was set as 40 seconds and the cycle time of each DDA cycle is set as 3 seconds. The MS2 Orbitrap resolution was set at 7,500 with standard AGC target and the maximum injection time were set at 1×10⁵ and 30 milliseconds, respectively.

Annexin-V apoptosis assay—Cells were stained by FITC-conjugated Annexin-V (BioVision) and propidium iodide (PI) (Invitrogen) in Annexin-V binding buffer (BD Biosciences) at room temperature for 30 minutes. Apoptosis percentage was determined using BD Accuri C6 flow cytometer and FACSDiva software (BD Biosciences).

STRING analysis—Protein candidates with unique peptide greater than 2 were input into the STRING platform for protein-protein interaction analysis.

Western blot analysis—Whole cell lysates were extracted using either NETN buffer supplemented with protease inhibitor cocktail or direct lysis. Protein lysate was separated by SDS-polyacrylamide gel electrophoresis (SDS-PAGE) and transferred to polyvinylidene difluoride membrane (Millipore) for western blot analyses. Primary antibodies FABP4[EPR3579](1:1000, ab92501, Abcam), pAKT(Ser473) (D9E), pGSK3β(Ser9) (D3A4), AKT and β-catenin (D10A8) (1:1000, #4060S, #9322S, #9272S & #8480S, Cell Signaling Technology), GSK3β(clone 7) (1:1000, #610202, clone 7, BD Transduction Laboratories), DDK (OTI4C5) (1:1000,TA50011-100, Origene), ITGB1 (1:1000, 12594-1-AP, Proteintech) and β-ACTIN (AC-74) (1:5000, #A5316, Sigma Aldrich) were incubated at 4° C. overnight. After washing, the membrane was incubated with horseradish peroxidase-conjugated anti-mouse or rabbit antibody (GE HealthCare). The signals were visualized using the enhanced chemiluminescence method. Blot images were quantified by densitometry using ImageJ software. The protein expression was normalized to β-actin and expressed as fold change relative to respective controls.

Immunohistochemical analysis of resected mouse tumours—Sections were deparaffinized in xylene and rehydrated in graded alcohols and distilled water. Slides were processed for antigen retrieval by a standard microwave heating technique in Tris-EDTA buffer. Endogenous peroxidase activities were quenched using 3% hydrogen peroxide. The sections were immersed in serum-free-protein block solution (DAKO). Specimens were subsequently incubated with primary antibodies (PCNA (PC10): 1:5000, ab29, Abcam; β-catenin (D10A8): 1:100, #8480S, CST). The sections were then washed thoroughly and incubated with anti-rabbit Envision HRP-conjugated secondary antibody (DAKO). Positive signals were visualized using Liquid DAB+ Substrate-Chromogen System (DAKO). Sections were counterstained with Mayer's haematoxylin followed by examination using light microscope. The number of positive PCNA nuclei and expression of β-catenin in mouse tumour tissue was quantified using ImageJ software.

Multiplexed fluorescent immunohistochemistry in human fatty liver tissue and resected mouse liver tumours—The tyramide signal amplification-based method was used for staining multiple targets in hepatocellular carcinoma paraffin embedding specimens with Opal 4-Color Manual IHC Kit (NEL810001KT, Akoya Biosciences). Sections were deparaffinized in xylene and rehydrated in decreasing graded alcohols and distilled water. Slides were processed for antigen retrieval by a standard inverter microwave heating technique with either diluted 50× Envision FLEX Target Retrieval Buffer (pH9.0, K8004, Dako) for FABP4, pGSK3β (Ser9), pAKT (Ser473), CD68, CD86 and CD206 or 10× AR6 sodium citrate buffer for Perilipin-1 and β-catenin (pH6.0, AR600250ML, Akoya BioSciences) for 15 minutes. Endogenous peroxidase activities were quenched using 3% hydrogen peroxide for 10 minutes at room temperature. The sections were immersed in blocking/antibody diluent (ARD1001EA, Akoya Biosciences) for 30 minutes at room temperature. Specimens were incubated with primary antibodies overnight at 4° C. (FABP4 [EPR3579] and Perilipin-1 (1:1000 for ab92501 and 1:200 for ab3526, Abcam), pGSK3β(Ser9) (D3A4), pAKT (Ser473) (D7F10), F4/80 (D2S9R), CD68 (E307V), CD86 (E5W6H) and CD206 (E6T5J) (1:500 for #9322S, 1:200 for #9018S, 1:200 for #70076T, 1:500 for #97778S, 1:500 for #19589S and 1:500 for #24595S, CST)). For β-catenin, primary antibody (D10A8, #8480S, CST) was incubated at 1:100 for 3 hours at room temperature. The sections were then washed thoroughly and incubated with Opal polymer HRP Mouse+Rabbit (ARH1001EA, Akoya Biosciences) for 30 minutes at room temperature. Followed by a brief wash with 1×TBST, Opal fluorophore (1:100) was applied for FABP4, pGSK3β(Ser9) or CD86 (Opal 520), Perilipin-1, pAKT (Ser473), F4/80 or CD68 (Opal 570) and β-catenin or CD206 (Opal 690) for 15 minutes at room temperature. A final stripping step was performed with diluted 10× AR6 sodium citrate buffer in the microwave oven for 15 minutes. The section slides were cooled down, counterstained with DAPI solution (1:1000) and mounted for examination using Leica TCS SPE confocal microscope. The fluorescent intensity was quantified using ImageJ software.

Beta-catenin TCF binding luciferase reporter assay—β-catenin activity was examined using luciferase reporter assay of TCF/LEF-dependent transcription (TOP/FOPFLASH reporter assay). Either firefly luciferase pSuper8XTOPflash or pSuper8XFOPflash constructs (gifts from Dr. Moon R, University of Washington, USA), together with Renilla luciferase construct pRL-CMV (Promega, Madison, WI, USA) for normalization of transfection efficiency, were transfected using Lipofectamine 2000. Luciferase activities were assayed using Dual-Luciferase Reporter Assay System (Promega) according to manufacturer's protocol.

Immunoprecipitation—The protein lysate was incubated with anti-DDK antibody (OTI4C5) (1:1000, TA50011-100, Origene), together with Protein A agarose beads (#9863, Cell Signaling Technology) at 4oC overnight with gentle agitation. Normal mouse IgG served as control. The immunoprecipitants were eluted by boiling in 2× SDS loading buffer for 10 minutes and then subjected to SDS-PAGE and immunoblotting analyses.

In vivo tumorigenicity assay—In vivo evaluation of tumorigenicity was performed with either male non-obese diabetic/severe combined immunodeficient (NOD/SCID) or NOD/SCID gamma mice, 4 to 6 weeks of age, by induction of tumour xenografts. Cells were suspended in 1:1 culture medium and BD Matrigel Matrix (BD Biosciences) and subcutaneously injected into the flanks of the mice, which were kept under observation. Briefly, each mouse received two injections of cells in both flanks. The cells were pretreated with either rhFABP4 (100 ng/ml) or ADCM for 24 hours, and, with their control group, were injected into NOD/SCID and NOD/SCID gamma mice, respectively. Tumours were harvested at the end of the experiment for documentation. Tumour-initiating cell frequency was calculated using Extreme Limiting Dilution Analysis (ELDA) software. No specific randomization method was used. Sample size of animals was chosen based on p-values. The tumour volumes did not exceed 10% of normal body weight or 1.3 cm in diameter. Mice were sacrificed if the percentage of body weight loss is greater than 20% including the tumour mass.

RNA extraction and quantitative PCR (qRT-PCR) analysis—Total RNA was isolated using TRIzol reagent according to the manufacturer's protocol (Invitrogen). Complementary DNA (cDNA) was synthesized using lug total RNA with PrimeScript RT Reagent Kit (Takara Bio, Shiga, Japan) according to the manufacturer's instructions and then subjected to qPCR with BrightGreen 2× qPCR Master mix (Applied Biological Materials Inc, Richmond, Canada) using QuantStudio 7 Flex Read Time PCR System (Applied Biosystems, Foster City, California, US) with primers specific to the sequences of genes of interest which were provided in Table 5. Relative expression differences were calculated using 2—ΔΔCTmethod with reference to either GAPDH or Actb.

RNA sequencing—Total RNA of rhFABP4 0 ng/mL (control) or 100 ng/ml treated PLC/PRF/5 and resected tumours from WT and Fabp4^{-/-} mouse in chemical induced high fat diet (HFD) model were extracted using TRIzol Reagent (Life Technologies) according to manufacturer's protocol. The quality of total RNA was checked by Agilent 2100 bioanalyzer (Agilent Technologies Inc.) to have OD260/280 ratio of between 1.8-2.0 and RNA integrity number (RIN) value higher than 8.0. The RNA samples which met the quality assessment were then subjected to Illumina sequencing using Hiseq 1500 sequencer (Illumina) or NextSeq2000 (Illumina) for performing sequencing run. Each pair-end sample had an average throughput of 20 Gb.

### TABLES

TABLE 1
Effect of adipocyte conditioned medium (ADCM) on tumorigenicity
of PLC/PRF/5 and Huh7 cells. (A) Subcutaneous in vivo tumour
development in NOD/SCID gamma (NSG) mice of pretreated
PLC/PRF/5 cells with either DMEM (control) or ADCM. (B)
Subcutaneous in vivo tumour development in NSG mice of
pretreated Huh7 cells with either DMEM or ADCM. Significance
was calculated by one-sided extreme limiting dilution analysis.
(A) Primary engraftment of PLC/PRF/5 cells

| | Tumor incidence rate | | | Extreme limiting dilution | | |
|---|---|---|---|---|---|---|
| | | | | Estimated | | |
| | 5 × 10³ | 1 × 10⁴ | 5 × 10⁴ | CSC | | |
| | cells | cells | cells | frequency | 95% CI | p-value |
| DMEM | 3/9 | 3/9 | 4/5 | 1/22710 | 1/11328- | 3.26e-06 |
| | | | | | 1/45528 | |
| ADCM | 9/9 | 7/8 | 5/5 | 1/2685 | 1/5262- | |
| | | | | | 1/1370 | |

(B) Primary engraftment of Huh7 cells

| | | | | Extreme limiting dilution | | |
|---|---|---|---|---|---|---|
| | Tumor incidence rate | | | Estimated | | |
| | 5 × 10³ | 1 × 10⁴ | 5 × 10⁴ | CSC | | |
| | cells | cells | cells | frequency | 95% CI | p-value |
| DMEM | 1/5 | 2/5 | 3/5 | 1/36748 | 1/88192- | 0.00902 |
| | | | | | 1/15312 | |
| ADCM | 3/5 | 3/5 | 5/5 | 1/8033 | 1/18175- | |
| | | | | | 1/3551 | |

TABLE 2
Key adipocyte-specific secretory proteins in ADCM determined by mass spectrometry. A
list of top 22 adipocyte-specific secretory proteins that were identified in ADCM was
shown. Score of zero is given to protein that was not detected in CAACM or HCCCM.

| | | Protein | | Fold Change | Fold Change |
|---|---|---|---|---|---|
| | | score | Protein score | (CAACM- | (HCCCM- |
| | Protein | (ADCM) | (CAACM) | ADCM)/ADCM) | ADCM)/ADCM |
| 1 | Laminin subunit | 913 | 810 | −0.11 | −0.55 |
| | gamma-1 (LAMC1) | | | | |
| 2 | Gelsolin (GSN) | 547 | 609 | −0.11 | −0.64 |
| 3 | Pentraxin-related | 494 | 231 | −0.53 | −1 |
| | protein PTX3 | | | | |
| | (PTX3) | | | | |
| 4 | Laminin subunit | 380 | 139 | −0.63 | −1 |
| | alpha-4 (LAMA4) | | | | |
| 5 | Tenascin C (TNC) | 353 | 23 | −0.93 | −1 |
| 6 | Lumican (LUM) | 258 | 266 | 0.031 | −0.90 |
| 7 | Annexin A5 | 253 | 353 | 0.39 | −1 |
| | (ANXA5) | | | | |
| 8 | Nidogen-1 (NID1) | 221 | 215 | −0.02 | −1 |
| 9 | Calmodulin (CALM) | 211 | 1218 | 4.77 | 1.72 |
| 10 | Legumain (LGMN) | 163 | 115 | −0.29 | −0.63 |
| 11 | Alpha-2-HS- | 126 | 214 | 0.69 | 1.31 |
| | glycoprotein | | | | |
| | (AHSG) | | | | |
| 12 | Periostin (POSTN) | 114 | 0 | −1 | −1 |
| 13 | Thymosin beta-10 | 95 | 98 | 0.03 | 2.66 |
| | (TMSB10) | | | | |
| 14 | Phospholipid transfer | 65 | 0 | −1 | −1 |
| | protein (PLTP) | | | | |
| 15 | Adipocyte enhancer- | 65 | 0 | −1 | −1 |
| | binding protein 1 | | | | |
| | (AEBP1) | | | | |
| 16 | Macrophage colony- | 59 | 69 | 0.17 | −1 |
| | stimulating factor 1 | | | | |
| | (CSF1) | | | | |
| 17 | Serglycin (SRGN) | 59 | 0 | −1 | −1 |
| 18 | Golgi membrane | 50 | 523 | 9.46 | 7.58 |
| | protein 1 (GOLM1) | | | | |
| 19 | Olfactomedin-like | 50 | 0 | −1 | −1 |
| | protein 3 (OLFL3) | | | | |
| 20 | Dickkopf-related | 40 | 0 | −1 | −1 |
| | protein 3 (DKK3) | | | | |
| 21 | Fatty acid-binding | 33 | 324 | 8.8 | −1 |
| | protein (FABP4) | | | | |
| 22 | Xaa-Pro dipeptidase | 27 | 76 | 1.8 | −1 |
| | (PEPD) | | | | |

TABLE 3
Effect of recombinant human FABP4 (rhFABP4) on tumorigenicity of
PLC/PRF/5 and Huh7 cells. (A) Subcutaneous in vivo tumour
development in NOD/SCID mice of pretreated PLC/PRF/5
cells with either PBS (control) or rhFABP4 at 100 ng/ml. (B)
Subcutaneous in vivo tumour development in NOD/SCID mice of
pretreated Huh7 cells with either PBS or rhFABP4 at 100 ng/ml.
Significance was calculated by one-sided extreme limiting dilution
analysis.
(A) Primary engraftment of PLC/PRF/5 cells

| | Tumor incidence | | Extreme limiting dilution | | |
|---|---|---|---|---|---|
| | rate | | Estimated | | |
| | 1 × 10³ | 5 × 10³ | CSC | | |
| | cells | cells | frequency | 95% CI | p-value |
| PBS | 2/5 | 1/5 | 1/8749 | 1/29109- | 0.0373 |
| | | | | 1/2630 | |
| rhFABP4 | 3/5 | 4/5 | 1/2108 | 1/5182- | |
| | | | | 1/858 | |

(B) Primary engraftment of Huh7 cells

| | | | | Extreme limiting dilution | | |
|---|---|---|---|---|---|---|
| | Tumor incidence rate | | | Estimated | | |
| | 1 × 10³ | 5 × 10³ | 1 × 10⁴ | CSC | | |
| | cells | cells | cells | frequency | 95% CI | p-value |
| PBS | 1/5 | 2/5 | 1/5 | 1/17193 | 1/47765- | 0.00435 |
| | | | | | 1/6189 | |
| rhFABP4 | 3/5 | 4/5 | 4/5 | 1/3404 | 1/7177- | |
| | | | | | 1/1615 | |

1. A monoclonal antibody which binds to fatty acid binding protein 4 (FABP4).

2. The monoclonal antibody of claim 1, wherein the monoclonal antibody binds to a sequence of FABP4 comprising SEQ ID NO. 1.

3. The monoclonal antibody of claim 1, wherein the monoclonal antibody binds to SEQ ID NO. 1.

4. The monoclonal antibody of claim 1, wherein the antibody is a human, humanised, or murine antibody.

5. The monoclonal antibody of claim 1, wherein the antibody is a neutralising antibody.

6. The monoclonal antibody of claim 1, wherein the antibody is an IgG, IgM, or IgA.

7. A monoclonal antibody obtained from a deposited hybridoma cell line.

8. A method for treating hepatocellular carcinoma in a subject, comprising administering an effective amount of a monoclonal antibody to the subject, wherein the monoclonal antibody specifically binds to fatty acid binding protein 4 (FABP4).

9. The method of claim 8, wherein the monoclonal antibody is a neutralising antibody.

10. The method of claim 8, wherein the antibody is a human, or humanised antibody.

11. The method of claim 8, wherein the monoclonal antibody binds to a sequence of FABP4 comprising SEQ ID NO. 1.

12. The method of claim 8, wherein the monoclonal antibody binds to SEQ ID NO. 1.

13. The method of claim 8, wherein the hepatocellular carcinoma is metabolic dysfunction-associated steatotic liver disease-related hepatocellular carcinoma (MASLD-HCC).

14. The method of claim 8, wherein the hepatocellular carcinoma is a result of metabolic-associated steatohepatitis (MASH).

15. The method of claim 8, wherein the monoclonal antibody is an IgG, IgM, or IgA.

16. The method of claim 8, wherein the effective amount is at least 30 μg per subject.

17. The method of claim 8, wherein administration of the effective amount of the monoclonal antibody to the subject results in one or more of the following:
Reduction in cell migration of hepatocellular carcinoma cells;
Reduction in cell invasiveness of hepatocellular carcinoma cells;
Reduction in self-renewal of hepatocellular carcinoma cells; and
Reduction in cancer stemness of hepatocellular carcinoma cells.

18. The method of claim 8, wherein the reduction is in comparison to the same feature as presented in a hepatocellular carcinoma cell prior to treatment.
